(19) United States
(12) Patent Application Publication
(10) Pub. No.: US 2016/0267809 A1
(43) Pub. Date: Sep. 15, 2016

(54) TECHNOLOGIES FOR BRAIN EXERCISE TRAINING

(71) Applicants: Christopher deCharms, Montara, CA (US); David Bressler, San Francisco, CA (US)

(72) Inventors: Christopher deCharms, Montara, CA (US); David Bressler, San Francisco, CA (US)

(21) Appl. No.: 15/160,041

(22) Filed: May 20, 2016

Related U.S. Application Data
(63) Continuation of application No. 14/790,371, filed on Jul. 2, 2015, now abandoned.
(60) Provisional application No. 62/156,853, filed on May 4, 2015.
(60) Provisional application No. 62/019,898, filed on Jul. 2, 2014.
(60) Provisional application No. 62/090,332, filed on Dec. 10, 2014.
(60) Provisional application No. 62/078,392, filed on Nov. 11, 2014.

(51) Int. Cl. G09B 19/00; A61B 3/113; A61B 8/08; G09B 5/06

(52) U.S. Cl. CPC G09B 19/00; G09B 5/065; A61B 3/113; A61B 8/0808

(57) ABSTRACT

A computer-implemented method of directing mental exercise includes providing, by a first output component, a stimulus representing an imagined perception, experience or activity that a user should attempt to generate in their mind; providing, by a second output component, an instruction for the user to perform a mental exercise that includes instructing the user to generate an internal felt sense of the imagined perception, experience or activity; receiving, at a user interface, an input that characterizes the user's internal felt sense, where the input includes an overt response from the user; determining, by a processing module, an attribute of the received input; determining, by the processing module and based on the determined attribute, a next instruction; storing at least one of the determined attribute and the determined next instruction in one or more memory locations; and training the user, including presenting the determined attribute, and providing the next instruction.

### CROSS-REFERENCE TO RELATED APPLICATIONS

This application claims priority to each of U.S. provisional application Ser. No. 62/156,853, titled “Methods for Online Training” and filed 4 May 2015, U.S. provisional application Ser. No. 62/090,332, titled “Methods for Online Training” and filed 10 Dec. 2014, U.S. provisional application Ser. No. 62/078,392, titled “Methods for Online Training” and filed 11 Nov. 2014, and U.S. provisional application Ser. No. 62/019,898, titled “Methods for Online Training” and filed 2 Jul. 2014, the entire contents of each of which is hereby incorporated by reference in its entirety.

### TECHNICAL FIELD

Described herein are methods, devices, computer-readable media, and systems for presentation of information and training and monitoring of users, and therapeutic and diagnostic applications.

### BACKGROUND

A variety of different approaches have been used to provide cognitive training to users.

### SUMMARY

In a first general aspect, a computer-implemented method of directing mental exercise includes providing, by a first output component of a computing device, a stimulus representing an imagined perception, experience or activity that a user should attempt to generate in their mind. The method also includes providing, by a second output component of the computing device, an instruction for the user to perform a mental exercise comprising instructing the user to generate an internal felt sense of the imagined perception, experience or activity. The method further includes receiving, at a user interface of the computing device, an input that characterizes the user's internal felt sense, the input comprising an overt response from the user. The method further includes determining, by a processing module of the computing device, an attribute of the received input, and determining, by the processing module of the computing device and based on the determined attribute, a next instruction. The method further includes storing at least one of the determined attribute and the determined next instruction in one or more memory locations of the computing device. The method further includes training the user, including: (i) presenting the determined attribute, and (ii) providing, by the second output component, the next instruction.

Various implementations can include one or more of the following. The stimulus may be an image, a video, a sound, or an animation. The input that characterizes the user's internal felt sense may characterize a time duration of the user's internal felt sense. The input that characterizes the user's internal felt sense may characterize an intensity of the user's internal felt sense. The input that characterizes the user's internal felt sense may characterize a satisfaction with the user's internal felt sense. The next instruction may be provided repeatedly with less than 30 seconds elapsing between repetitions. The input that characterizes the user's internal felt sense may be received at the user interface as a selection of one or more buttons, a position of one or more sliders, one or more form input elements, a cursor position, a touch screen position, voice recognition, or one or more eye movements. The method may further include receiving, at the user interface, an input that characterizes the user, and selecting, based on the received input that characterizes the user, the stimulus from a plurality of predefined stimuli. The instruction for the user to perform a mental exercise may be configured to decrease pain. The instruction for the user to perform a mental exercise may be configured to decrease pain, decrease stress, treat depression, treat anxiety, treat addiction, treat insomnia decrease craving, increase attention, increase relaxation, increase happiness, increase focus, or increase learning. The mental exercise may be capable of remaining internal to the mind of the user. The attribute may include a score. The method may be used with a medication therapy. The method may further include testing the mental exercise in combination with a plurality of medications, and identifying a particular medication from the plurality of medications to associate with the mental exercise. The user's internal felt sense may include a mental image. The mental exercise may include mental rehearsal. The imagined perception, experience or activity may include a first aspect followed in time by a second aspect, and the instruction for the user to perform a mental exercise may include the instruction to generate the first aspect of the internal felt sense of the imagined perception, experience or activity, and then the second aspect of the internal felt sense of the imagined perception, experience or activity. The method may further include providing, on a display screen of the computing device, a moving object, wherein motion of the object is configured to guide timing of the mental exercise. Each of the stimulus, instruction, and the mental exercise may be derived based on brain imaging information. A time between the first aspect and the second aspect may be less than 10 seconds. The method may further include determining, by the processing module of the computing device and based on the determined attribute, a next stimulus and providing, by the first output component, the next stimulus. The method may further include receiving a user indication of a medication, and selecting the stimulus and the instruction for the user to perform a mental exercise based on the medication. The method may further include receiving a user indication of a medical condition, and selecting the stimulus and the instruction for the user to perform a mental exercise based on the medical condition. The medical condition may be gabapentin. The method may further include receiving an input that specifies a medication taken by the user, and the determining the next instruction may be based in part on the medication. The method may further include instructing the user to generate an imagined tactile experience. The method may further include receiving an input that specifies a medical or psychological condition of the user, and the determining the next instruction may be based in part on the medical or psychological condition.

In a second general aspect, a computing device for directing mental exercise includes, a first output component configured to provide a stimulus representing an imagined perception, experience or activity that a user should attempt to generate in their mind. The computing device also includes a second output component configured to provide an instruction for the user to perform a mental exercise comprising instructing the user to generate an internal felt sense of the imagined perception, experience or activity. The computing device further includes a user interface configured to receive an input that characterizes the user's internal felt sense, the input comprising an overt response from the user. The computing device further includes a processing module configured to: (i) determine an attribute of the received input, (ii) determine, based on the determined attribute, a next instruction, and (iii) train the user, including: (i) causing the determined attribute to be presented, and (ii) causing the next instruction to be provided by the second output component.

In a third general aspect, a computer-implemented method of directing mental exercise includes providing, by a first output component of a computing device, a stimulus representing an imagined perception, experience or activity that a user should attempt to generate in their mind. The method also includes providing, by a second output component of the computing device, an instruction for the user to perform a mental exercise comprising instructing the user to generate an internal felt sense of the imagined perception, experience or activity. The method further includes receiving, at a user interface of the computing device, an input that characterizes the user's internal felt sense, the input comprising an overt response from the user. The method further includes determining, by a processing module of the computing device, an attribute of the received input, and determining, by the processing module of the computing device and based on the determined attribute, a next stimulus. The method further includes storing at least one of the determined attribute and the determined next stimulus in one or more memory locations of the computing device. The method further includes training the user, including: (i) presenting the determined attribute, and (ii) providing, by the first output component, the next stimulus.

In a fourth general aspect, a computer-implemented method of directing mental rehearsal includes receiving, at a user interface, an input about a user, and selecting, by a content engine, a particular stimulus representing an imagined perception, experience or activity that a user should attempt to generate in their mind, where the particular stimulus is selected from a plurality of predetermined stimuli. The method also includes providing, by a first output component of a computing device, the selected stimulus representing an imagined perception, experience or activity that a user should attempt to generate in their mind. The method further includes receiving, at a user interface of the computing device, an input that characterizes the user's imagined perception, the input comprising an overt response from the user. The method further includes determining, by a processing module of the computing device, an attribute of the received input, and determining, by the processing module of the computing device and based on the determined attribute, a next stimulus. The method further includes storing at least one of the determined attribute and the determined next stimulus in one or more memory locations of the computing device. The method further includes training the user in mental rehearsal, including: (i) presenting the determined attribute, and (ii) providing, by the second output component, the next stimulus. Other features, objects, and advantages of the technology described in this document will be apparent from the description and the drawings, and from the claims.

### BRIEF DESCRIPTION OF THE DRAWINGS

FIG. 1 is an example overview diagram.

FIG. 2 is an example training screen.

FIG. 3 is an example settings screen.

FIG. 4 is an example mental state input screen.

FIG. 5 is an example multiple state input screen.

FIG. 6 is an example slide out menu and home screen.

FIG. 7 is an example home screen on mobile device.

FIG. 8 is an example level selector screen.

FIG. 9 is an example pacing screen.

FIG. 10 is an example paintone screen.

FIG. 11 is an example journal screen.

FIG. 12 is an example progress and statistics screen.

FIG. 13 is an example reminders screen.

FIG. 14 is an example profile screen.

FIG. 15 is an example basic loop.

FIG. 16 is an example flowchart.

FIG. 17 is an example combination treatment flowchart.

FIG. 18 is an example involving physiological measurement.

### DETAILED DESCRIPTION

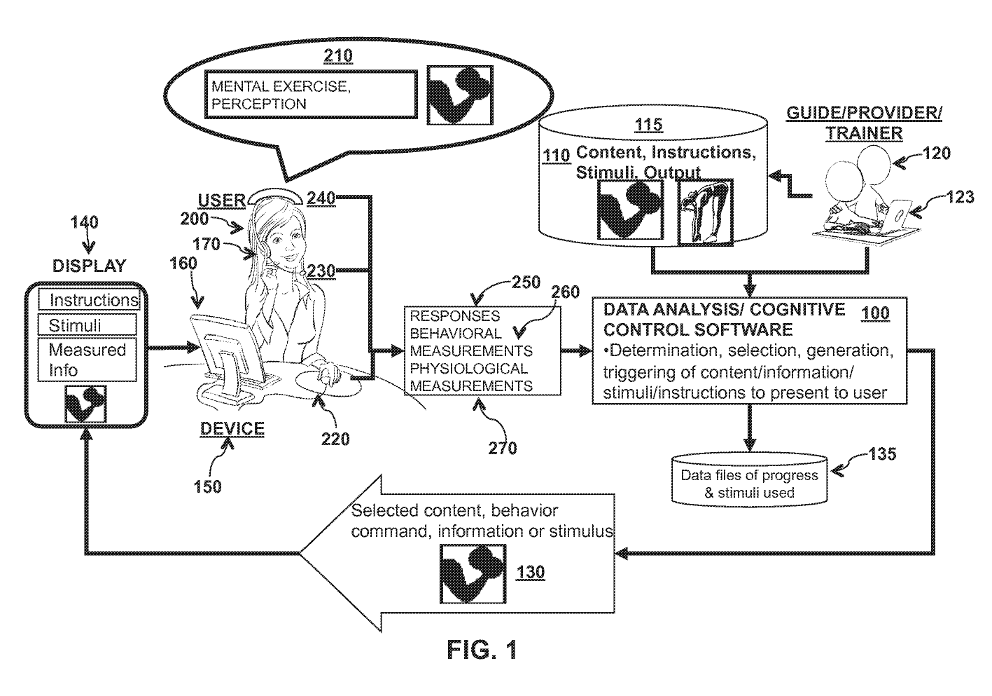

The present methods, devices, software and systems provided herein may be used to provide and enhance the activation and control of brain activation, mental and cognitive activities, performance and symptoms. An overview diagram depicting the components and process of the methods, devices, software and systems provided herein is presented in FIG. 1.

### Embodiment Example 1

### Training

The user may download an software on their mobile device or computer, or accesses it via the internet or wireless. The software may provide an introductory video explaining what it is useful for, for example explaining that the software may be used to learn mental exercises to decrease physical pain. The user may then use the software to further characterize themselves, for example, they may answer questions through the software or provide information about themselves. This information may be used to make later determinations of content personalization for the user. The user, or the app, or the user's guide or provider, may select a content module that the user may engage in. The content module may provide a series of exercises designed to be beneficial to the user. As an example, the content module may be designed to decrease the user's pain. The content module may be designed to teach a user to control elements of their cognitive, mental, physical, physiological, or neurophysiological functioning, for example to achieve this goal, according to some examples. The content module may be designed to teach a user to engage in specific mental exercises designed to engage the antinociceptive system in the brain, and thereby produce decreases in pain over time, according to some examples.

The user may be offered a variety of difficulty levels of training, based on their skill level and progress. The user may select a level, for example by clicking an icon on their device screen that may indicate the name or type of training that they will receive or show an image indicating its purpose or nature.

Once the user selects a level (or the software selects one for them based on their progress to that point), the user may be provided with a programmed sequence of one or more instructions, or stimuli intended to convey something that the user should do. In this simple example, the user may be provided with an instruction to engage in a sequence of two alternating mental exercises, each exercise designed to engage the brain's antinociceptive system, and thereby to decrease the user's pain. For example, the user may be instructed to focus on an area of their body where they feel pain, and then to imagine the tactile sense that would be produced if they were feeling warm water on this area of their body. The user may be instructed to focus their attention on these imagined warm sensations. The user may be instructed to intentionally create sensations in this area of their body, for example the sensation of warmth.

The user may be provided with the instruction to focus their attention on assessing the internal felt sense (e.g., mental image, visualization, or imagined sensation) that they have created. For example, the user may be instructed to quantitatively or qualitatively assess their internal subjective experience as they perform this exercise. The user may assess their experience in a variety of ways, including for example noting any or all of their experience's: duration, vividness, specificity, intensity, imagined physical extent (e.g. size/shape), qualities (e.g. degree of temperature, tingling, burning, pulsing), their success at producing the experience, how much they like or don't like the experience, the extent to which the experience improves or worsens what they intended to improve (e.g. their pain), their emotional response to the experience, their physical response to the experience, or a variety of other assessments.

The user's assessments of their experience may be inputted into the user interface of a computing device, and the computing device may receive the input. The user's input information may be entered in a large variety of ways. For example, the user may indicate the degree to which they were able to successfully create the sensation of warmth, indicating this to the UI by using UI elements such as the selection of buttons, sliders, form input elements, cursor or touch screen position, voice recognition, eye movements meant to indicate this, or other UI input approaches.

In this manner, the user may provide the input by an overt response from the user. For example, the user may provide an input entry at a user interface of the computing device in any of the ways discussed above or others. This is different than an example where one or more biofeedback or neurofeedback signals from the user are collected and/or measured. In this example, the user is purposely providing feedback on the mental exercise based on the user's subjective interpretation of the internal felt sense experienced by the user in following the instruction. In this example embodiment, the user's own self-awareness or subjective experience can be employed to assess the results of their internal mental exercise, rather than using a physiological measurement.

The user's assessments of their internal mental exercise and internal felt sensations that result from it may take a very simple and concrete form, for example the user may indicate with a button press on the UI how long they spent performing the mental exercise of imagining warmth in this part of their body, by clicking the button when they are done (or at some other known or estimated intervening point in-) performing the mental exercise. The assessment may also take more complex forms, or forms with a more subjective character, such as the user indicating the vividness or perceived temperature of the imagined warmth. These assessments may provide an indication of the internal mental or subjective activities of the user.

The assessments may at this point, following in time after an individual stimulus, be stored to computer memory or storage, and may be used to determine what happens next, or what stimuli are presented next, or when (or if) they are presented. This may happen continuously, forming a recurring loop, or a feedback loop, in some examples.

In some examples, the computing device or system may present a next stimulus may be provided to the user. For example, a second stimulus designed to form a pair or sequence may be provided. Following the instruction to form a mental image of warmth in a body part where the user is experiencing pain, the device or system may provide a stimulus of an instruction to form a mental image of the tactile sensation of cool in the body part where the user is experiencing pain. This may proceed as described above for the instruction to produce a mental image of warmth. It also may proceed through a step involving the user assessing their resultant experience.

The computing device or system may present stimuli in a sequence. For example, the device or system may provide the user with the stimulus of the instruction to perform the mental exercise of creating a sense of warmth in a body area, followed at a later time by presenting the stimulus of the instruction to perform the mental exercise of creating a sense of coldness in a body area (e.g., the same body area or a different body area). The stimuli of this sequence, a sequence of two stimuli in this case, may be repeated. For example, the device or system may provide alternating instructions to create a mental image of warm, and then cold, and the user may follow the instructions. After each individual stimulus or instruction, or at some point in the sequence, or after the completion of the sequence of stimuli, the device or system may instruct the user may to make assessments as described above. In some examples, the instruction to make the assessments may be provided in advance of the entire sequence, or may be provided during the sequence, or may be made following an individual stimulus.

The assessments may be received as input at a user interface (UI) by the device or system at this point, following in time after a sequence of stimuli, may be stored to computer memory or storage, and may be used to determine what happens next, or what stimuli are presented next, or when (or if) they are presented, according to some examples. This may happen continuously, forming a recurring loop, or a feedback loop.

For example, the user's input to the UI may indicate the timing of their completion of the mental task of imaging warmth, and then alternately of imagining cool. The user may also input how effective they were in imagining warm or cool. The device or system may receive this input from the user, for example, and may determine one or more attributes or characteristics of the input.

The device or system may use the input information from the user to determine a score for the user. For example, for each trial or session or portion of a trial or session, the device or system may determine or calculate a score based on the input received from the user. For example, the user may be scored based on how evenly timed their input is. In this example, the user may receive points based on how closely matched the duration of each mental exercise that they perform is to the timing rhythm including a pre-selected duration, or to a timing rhythm that the user has established themselves, for example through the timing or duration of past warm/cool sequences. The user may also receive points and be provided with a stimulus including information indicating when their timing is sufficiently close to the target timing to achieve a ‘hit’, or be denied points if their timing is not sufficiently close, in which case they may be informed of a ‘miss’.

The device or system may provide such information in a variety of ways. Information on their score may be provided numerically, for example by providing a score, or a number of hits and misses, or by providing icons or graphic images indicating their score on a display device, for example. The device or system may also present information to indicate a user's success may also by one or more sounds (e.g., via one or more speakers), such as a sound that is presented for ‘hits’ and a different sound for ‘misses’, or a sound whose parameters (such as pitch, volume, duration, selected sound file contents) are based on the user's timing accuracy. The user's score may also be based on other elements of their assessment of their experience, such as their assessment of their ability to successfully perform the instructed mental exercise, or the quality of the performance that they perceive that they achieved (for example none, low, medium, high). The user may receive separate scores for any of the different assessments that they input, or for combinations. In some examples, the device or system may compute a score based on two or more determined attributes or characteristics. For example, a score representing a product of (e.g., multiplicative product of) the duration of their mental exercise, the accuracy, and their perception of their success, each weighted by an appropriate factor. The user's score may then be stored, summed across trials and/or sessions, compared with scores from other users, and in other respects used to make the process more enjoyable, challenging, and motivating.

The device or system may use input from the user to control many aspects of the user's experience. For example, the user may select the rate, timing or rhythm at which instructions are provided, or at which they perform the mental exercises. The device or system may provide interface features that permit the user to self-pace the mental exercises, and may score the user based on their ability to keep an even timing (e.g., determined from the input received from the user), consistent across trials. The device or system may also provide options to permit users to be trained using fixed timing of various pacing. Users may also used fixed timing using timing parameters derived from or based on preferences or testing with previous users, according to some examples.

On successive trials, users may receive the same or different instructions, according to various implementations. For example, on each successive trial, the device or system may present the user with different instructions for how to perform the task of internally creating a warm sensation. For example, the user may be instructed to imagine the tactile feeling of warm water, or a warm pack, or a warm bath, or a warm stone, or a warm hand, or a warm heating pad, or an inner sense of warmth. On cold trials, the user may be instructed to imagine the tactile feeling of cool water, or a cold pack, or a cold bath, or a cold pad.

As the device or system receives user input regarding their results with each instruction, the device or system may use the inputs to determine future instructions to be provided to the user by the device or system. For example, the user may rate which instructions are the most successful or desirable for them. This information may be stored. This information may be used to preferentially select preferred instructions at a later time, or to avoid less preferred instructions. As another example, instructions that users are more successful at using may be provided in early phases of training, and instructions that users are less successful at using may be provided in later phases of training. Inference algorithms, for example Bayesian inference, may be used to determine which stimulus or instruction to present to the user on each trial based on which stimuli or instructions have been most successful for the user, and/or which stimuli or instructions have been most successful for previous users, and/or which stimuli or instructions have been most successful for previous users with one or more similar characteristics to the current user. For example, this similarity may be based on similarity of answers to characterization questions answered by the user, by the user's pattern of choices in performing the training, or by the user's success in performing the training. For example, stimuli or instructions for the current user may be selected based on their expected success determined by their level of success in prior users who selected other stimuli or instructions similar to the pattern selected by the current user, or who had a similar pattern of success or assessments of stimuli or instructions relative to the current user.

Through ongoing practice of the exercises presented, users may experience decreases in their ongoing level of pain (or other undesirable symptoms or states) and increases in their ability to control their pain (or other undesirable symptoms or states). This may lead to long term plasticity, neuroplasticity, and long-term changes in the user's abilities and improved status.

### Embodiment Example 2

### Breathing

The user may download an software on their mobile device or computer, or access it via the internet or wireless. The software may provide an introductory video explaining what it is useful for, for example explaining that the software may be used to learn mental exercises to increase relaxation or decrease anxiety or depression. Many of the additional steps described in Example 1 & 3 may also be used, though they may not be repeated in this section. The user may select a content module that the user may engage in that may provide a series of exercises designed to be beneficial to the user. As an example, the content module may be designed to increase relaxation or control over breathing, or decrease anxiety or depression. The content module may be designed to teach a user mental exercises following sequences and/or control over breathing.

Once the user selects a level (or the software selects one for them based on their progress to that point), the user may be provided with a programmed sequence of instructions, or stimuli intended to convey something that the user should do. In this simple example, the user may be provided with the instruction to engage in a sequence of two alternating mental exercises, and/or the user may be instructed to concurrently breathe in on one phase of the sequence and out on the next (or to breathe in and out on each phase). The timing of the sequence of the instructions may be provided by the software, or may be controlled by the user, for example by clicking the UI to receive each additional sequence step.

The software may provide a sound stimulus accompanying each step. For example, during the ‘inhale’ phase, a corresponding sound may be played, and during an ‘exhale’ phase a corresponding sound may be played. The sequential timing may be controlled at the level of the sequence by the software, for example by repeating a fixed duration of each sequence step. The timing may be controlled by the user, for example by selecting one of multiple fixed durations (e.g. fast, medium, slow). The timing may be controlled by the user by self-pacing: for example, each time the user clicks the UI to indicate that they have completed a sequence step, this may allow the software to determine that it is time to present the next step, optionally after a delay. The timing accuracy of the user may be measured. For example, a ‘target’ duration for each sequence step or for each sequence cycle may be established by the software or by the user. The user may indicate when they have completed each sequence step (and/or which step they have completed) or each cycle. The time that they indicate this may be compared with the target time or duration. The user may be presented with information or stimuli based upon the determination of the relationship between the user's time and the target time. For example, if the user's time is within a certain percent difference from the target time, the user may be presented with one sound stimulus, and receive one level of points or score, while if the user's time is within a different (e.g. larger) percent difference from the target time, the user may be presented with a different sound stimulus, and receive a different level of points or score (e.g. lower or none). Several levels of accuracy may be used to achieve a target with different point values, or a continuum of scoring. The user's score may also be based upon multiple factors, including their timing accuracy, the duration of the sequence step or cycle, and their performance of the mental task.

The assessments may at this point, following in time after an individual stimulus, be stored to computer memory or storage, and may be used to determine what happens next, or what stimuli are presented next, or when (or if) they are presented. This may happen continuously, forming a recurring loop, or a feedback loop.

### Breathing Measurement Device

A user may use the microphone of a device, for example a mobile phone, to measure their breathing, using the sound input created by breathing to indicate when breathing is occurring. For example, a user may use mobile phone ear bud headphones (e.g. iPhone earbuds or custom-built headphones for the purpose) in a configuration that places the microphone close to the nose or mouth of the user. For example, if the user hangs the cord of the right side earbud (which includes the microphone) over the left ear while keeping the right earbud in the right ear, then the microphone may hang across the face close to the nose and mouth, and be able to pick up breathing-related sounds.

### Breathing-Based Feedback and Timing or Scoring

The user may use a measurement device that is able to measure their breathing for use during training. The software may use measures of the user's breathing from this device in place of or in addition to the user's inputs to a UI to determine the accuracy of the timing of the user's breathing, or of a mental exercise that the user is instructed to perform in time with their breathing. In this way, the user may use their breathing as an indicator of the timing of their mental activity.

The software may provide auditory feedback of the user's recorded breathing. For example, the audio signal of the user's breathing may be inputted by the software, amplified and played back to the user in substantially real time, so that the user can hear their own breathing. This can be helpful in maintaining awareness of the breath. In addition, the sounds input by the system may have sound effects applied to them, prior to being played back to the user. The sound effects include any available on digital effects pedal software and similar processors, including flange, delay, feedback, and others. The sound may also be visualized on the screen or UI for display to the user in substantially real time. This may include real time displays of: the sound waveform, the sound spectrum, the sound spectrogram, or animated visualizations driven by the sound. The software may allow for additional plug-ins for further visualization effects.

The software may use the level of the sound or level of the sound in different frequency bands or pattern recognition or speech recognition algorithms. These algorithms may determine when the user breathes in, breathes out, or completes a breathing cycle. These times may be used to indicate when the user has completed each sequence step or instruction, or a cycle of sequence steps or instructions. In this way, the user's breath may be used as a game controller. In addition, the user may use speech commands, hums, clicks, or other sounds that they are able to produce to control aspects of the game or issue commands to the software.

### Embodiment Example 3

### Learning Timing Rhythm

The user may download an software on their mobile device or computer, or access it via the internet or wireless. The software may provide an introductory video explaining what it is useful for, for example explaining that the software may be used to learn mental exercises based on a timing rhythm. Many of the additional steps described in Example 1 & 2 may also be used, though they may not be repeated in this section. The user may select a content module that the user may engage in that may provide a series of exercises designed to be beneficial to the user. As an example, the content module may be designed to increase the user's ability to perform mental exercises, and/or to improve the rhythm of their timing. The content module may be designed to teach a user mental exercises following sequences, including timed sequences.

The user may be provided with a programmed sequence of instructions, or stimuli intended to convey something that the user should do. In this simple example, the user may be provided with the instruction to engage in a sequence of two alternating mental exercises. The timing of the sequence of the instructions may be provided by the software, or may be controlled by the user, for example by clicking the UI to receive each additional sequence step, using a sound or voice control or other input. This input may indicate when the user has completed each sequence step, or when they are ready to go to the next step.

The software may provide a sound stimulus accompanying each step. For example, during one phase, a corresponding sound 1 may be played, and during a second phase a corresponding sound 2 may be played. The sequential timing may be controlled at the level of the sequence by the software, for example by repeating a fixed duration of each sequence step. The timing may be controlled by the user, for example by selecting one of multiple fixed durations (e.g. fast, medium, slow). The timing may be controlled by the user by self-pacing: for example, each time the user clicks the UI to indicate that they have completed a sequence step, this may allow the software to determine that it is time to present the next step, optionally after a delay.

The timing accuracy of the user may be measured. For example, a ‘target’ duration for each sequence step or for each sequence cycle may be established by the software or by the user. The user may indicate when they have completed each sequence step (and/or which step they have completed) or each cycle. The time that they indicate this may be compared with the target time or duration. The user may be presented with information or stimuli based upon the determination of the relationship between the user's time and the target time. For example, if the user's time is within a certain percent difference from the target time, the user may be presented with one sound stimulus, and receive one level of points or score, while if the user's time is within a different (e.g. larger) percent difference from the target time, the user may be presented with a different sound stimulus, and receive a different level of points or score (e.g. lower or none). Several levels of accuracy may be used to achieve a target with different point values, or a continuum of scoring. The user's score may also be based upon multiple factors, including their timing accuracy, the duration of the sequence step or cycle, and their performance of the mental task.

The assessments may at this point, following in time after an individual stimulus, be stored to computer memory or storage, and may be used to determine what happens next, or what stimuli are presented next, or when (or if) they are presented. This may happen continuously, forming a recurring loop, or a feedback loop. The software may also include the device and/or steps based on breathing as described in Exercise 2.

The software may use the user's input, for example a UI click, or the level of the sound or level of the sound in different frequency bands or pattern recognition or speech recognition algorithms. These times may be used to indicate when the user has completed each sequence step or instruction, or a cycle of sequence steps or instructions. In this way, the user's breath may be used as a game controller. In addition, the user may use speech commands, hums, clicks, or other sounds that they are able to produce to control aspects of the game or issue commands to the software.

### FIG. 1. Example Overview Diagram

As illustrated, control software 100 may initiate and/or control cognitive training sequences. The software may select stimuli or instructions 110 from a database 115 of previously saved content. Instructions or stimuli may also be provided from another individual, such as a trainer 120, who may work on a remote computer 123. The instructions provided by the trainer may be recorded, edited, and stored in the database 115.

The control software may perform determination and/or selection, generation, and triggering of information such as stimuli or instructions to be presented to the user 200. The results and other information and ongoing collected data may be stored to data files of progress and a record of the stimuli used 130 in a database 135. The determined or selected instruction, measured information, or stimulus display 140 or output, may then be presented via a device 150 using a display 160 or headphones 170 (or headset/speakers) or other output to a user 200. This may instruct the user to engage in imagined or overtly performed behaviors or cognitive or mental exercises 210 or to perceive stimuli. If the user undertakes overt behaviors, such as responding to questions, the responses and other behavioral measurements may be recorded using an input device 220 (such as a mouse, keyboard, touchpad, etc.) or by speech using a microphone 230.

In addition, information may be collected from sensors 240 positioned about the user. These sensors may measure physiological activity (fMRI, realtime fMRI, MEG, fUS (functional ultrasound), fNIRS, EEG, EMG, heart rate, skin conductance, breathing, eye movements, movement) or behaviors. In this case, this information may be used in the context of biofeedback.

Through the use of the present methods, devices, software and systems provided herein, a user may be able to be trained to control the activation of that user's brain, and to perform mental or cognitive exercise to further increase the strength and control over activation or mental or physical performance. This training and exercise may have beneficial effects for the user. In the case that regions of the brain may release endogenous neuromodulatory agents, this control may serve a role similar to that of externally applied drugs, such as in decreasing pain, controlling depression or anxiety. There may also be applications in learning, focus, and performance enhancement.

The mental exercises, mental rehearsal, or exercises of brain regions of interest according to the present methods, devices, software and systems provided herein may be analogous to the exercise provided by specialized training equipment for weight lifting that isolates the activation of a particular set of muscles in order to build strength and control in those muscles.

In addition to training and exercise, knowledge of the activation pattern in discrete brain regions may be used to enhance certain aspects of a user's behavioral performance, such as the user's abilities at mental exercises, mental rehearsal, perception, learning and memory, and motoric skills. This enhancement takes place by cueing a user to perform a behavior at a point when a measured pattern of brain activation is in a state correlated with enhanced performance. Alternatively, the behavior that the user may undertake or the stimulus that the user may perceive may be selected based upon the measured pattern of neural activation from this user or prior users.

As may be explained herein, any brain measurement methodology may be used in conjunction with the present methods, devices, software and systems provided herein, or as a sensor 240. The physiological activity of the brain may be effectively monitored, especially in substantially real time. In one embodiment that may be described in greater detail, the brain scanning methodology used is functional magnetic resonance imaging (fMRI).

The responses 250, behavioral measurements 260, or physiological measurements 270 of the user may be captured and recorded by the control software. For example, the user's responses in response to, or during, or following an instruction may be captured. In addition, behavioral measurements may be captured, for example the users EEG or EMG. In addition, the users spoken responses or commands may be captured.

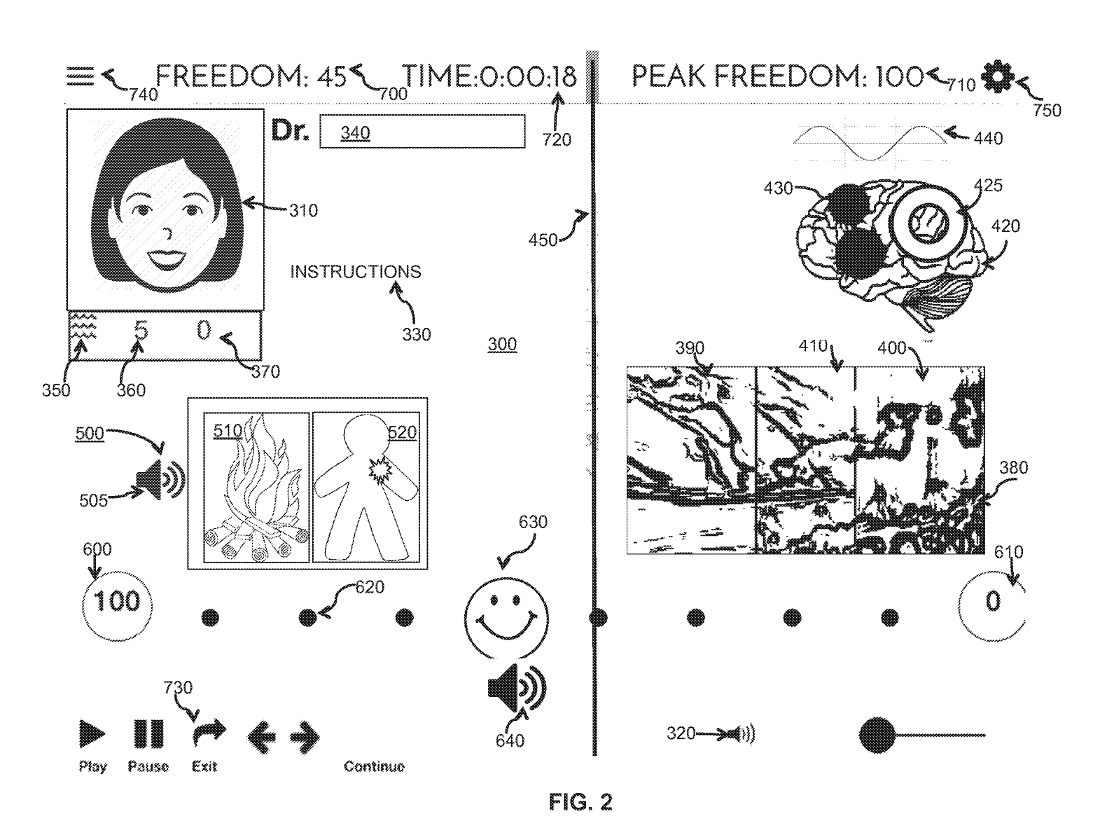

### FIG. 2. Example Training Screen

The training screen 300 may be presented on a device such as a computer or mobile device or VR or AR device to the user. The training screen may provide many elements for stimuli, instruction and information for the user, as well as to accept user input. These elements may be provided in different sizes, locations or combinations than shown. The training screen may be used to guide the user through training. All elements of this screen may be presented by the control software.

A trainer or guide 310 may be presented visually as an image, photo, icon, or using live or pre-recorded video. This may be pre-recorded, or live, for example by videoconference. Also, audio 320 may be presented. The audio may include instructions 330 from the trainer. The trainer's name, avatar or handle 340 may be provided.

The trainer (directly or pre-recorded via the control software) may provide stimuli mental exercises, cognitive exercises, and brain ‘postures’ 350, such as the water posture (icon shown), which may correspond to sets of instructions to be performed by the user. The software may serve as the trainer or guide, or the guide may work through the software, or the two may work together to select the stimuli, content or instructions presented to the user. These may be provided in sequences. Each individual instruction may be presented and its number in the sequence shown 360. In addition, a countdown timer 370 may show how long the user has been engaged with an instruction, posture, or sequence. The overall training time 720 may also be presented and maintained.

The screen may also include content 380, including video, virtual reality or augmented reality content. For example, the screen may include video1 390 and video2 400, which may be controlled in their opacity and superimposed on top of one another 410 to provide an image with combined content. In the case shown, the middle panel depicts a semi-transparent combination of video1 and video2. This content may be provide as a background behind other screen elements, and may be provided as a full-screen background, or may be provided to fill certain panels or sections of the screen. In the case shown, the content takes up only a small portion of the screen for clarity of the other elements.

The screen may also include a depiction of the brain, 420. This depiction may include images, video, transparency, animation, or brain activation depictions computed in real time by the control software. The depiction may show patterns of brain activation 430, for example by showing different colors that depict different levels of activation in different areas of the brain. The screen may also show static or moving graphs 440 representing real or simulated physiological signals including brainwaves. The software may also present target brain regions 425 for control, or target brain activation patterns to produce or inhibit. These graphs may also be used to depict the user's progress or behavior. In one implementation, the graph depicts the user's real time ratings. For example, the color, amplitude, frequency, thickness or other elements of the graph may be modulated in accordance with the user's input by the software.

The user's input may be captured using an input device such as a mouse position, slider, mouse movement, mouse click, touchpad position, touch movement, touch up or down, keyboard, movements measured by accelerometer, sound measurements from the user including measuring the sound of the users breath or heart captured through a microphone, spoken commands or responses by the user through a microphone or others. In the example shown, the large vertical line 450 slides left and right with the user's mouse or touch screen position, similar to a slider. The user is instructed to be aware of their own internal mental state, and to indicate its magnitude by the position of this slider. For example, the user may be instructed to be aware of the level of their pain sensation, and to indicate this pain level continuum with the position of the line on the screen, the left most point on the screen corresponding to the most pain, and the right most point on the screen corresponding to the least pain (or vice versa).

Stimuli, instructions or feedback 500 to the user which may be determined based upon the user's input and other factors may be presented in real time based upon the user's actions. This feedback may take a variety of forms, including but not limited to: volume of a sound 505 (e.g. the sound of water corresponding to the video of water playing in the foreground on the screen), opacity or intensity of a visual stimulus (e.g. the opacity of video1 of water), nature of content being presented (e.g. control a VR or AR scene being generated for the user based upon the user's inputs), or qualitative video or animated elements of stimuli presented (e.g. how fast the water is flowing in a video or animation). Two cartoon representations of types of feedback are presented in the figure. 510 depicts an animated or virtual reality depiction of a fire, in which the magnitude of the fire may be controlled by the user's input. For example, as the user selects inputs to the left, which may represent greater pain, the fire becomes larger (as shown) and is animated to burn vigorously and make loud fire noises. If the user selects inputs to the right, which may represent less pain, the fire becomes much smaller, and produces less sound (not shown). In another example, 520 depicts an animated or VR representation of a human body, where an area of the body which may be indicated to be in pain, and may be selected by the control software to correspond to one or more areas where the user has indicated that they experience pain, is controlled by the user. The area indicated to be painful may grow or change color or pulse as the user selects to represent greater pain, and may become smaller as the user selects to indicate lesser pain (e.g. selecting right side of screen). The software may also allow the user to ‘add’ areas to the image indicate where they experience pain.

The screen may include an indication of the user's starting level 600 and/or an indication of the user's target level 610, and or intermediate targets 620 representing points in between. These values may represent pain, or another aspect that the user may intend to control. The value for the starting level, target level, or intermediate targets may be set by or input by the control software based upon previously input values from the user, for example using prior screens.

When the user slides the vertical bar 450 to the right, or otherwise indicates that their experience or sensation (e.g. pain) has decreased below each intermediate target level or the final target, the software determining that the user is hitting these targets may produce visual rewards 630 or sounds 640 to indicate success to the user. This may be determined based upon both the magnitude of change achieved by the user and the time that the magnitude has been maintained by the user in order to determine a success indicator, such as a score. For example, the user may be required to have pain less than a given level for a selected period of time in order to achieve each new level and be rewarded. Upon reaching each intermediate level, the user may receive an increment to an indication of their score, which may be indicated 700. In addition, their peak performance 710 and time spent 720 may be measured and presented. The user may use controls 730 to play or pause the progress, exit, or move to the next or previous instruction or screen, or to restart an exercise (not shown). In addition, the user may select a menu button 740 to go to the home screen or control more options, or a settings button 750 to go to a settings screen.

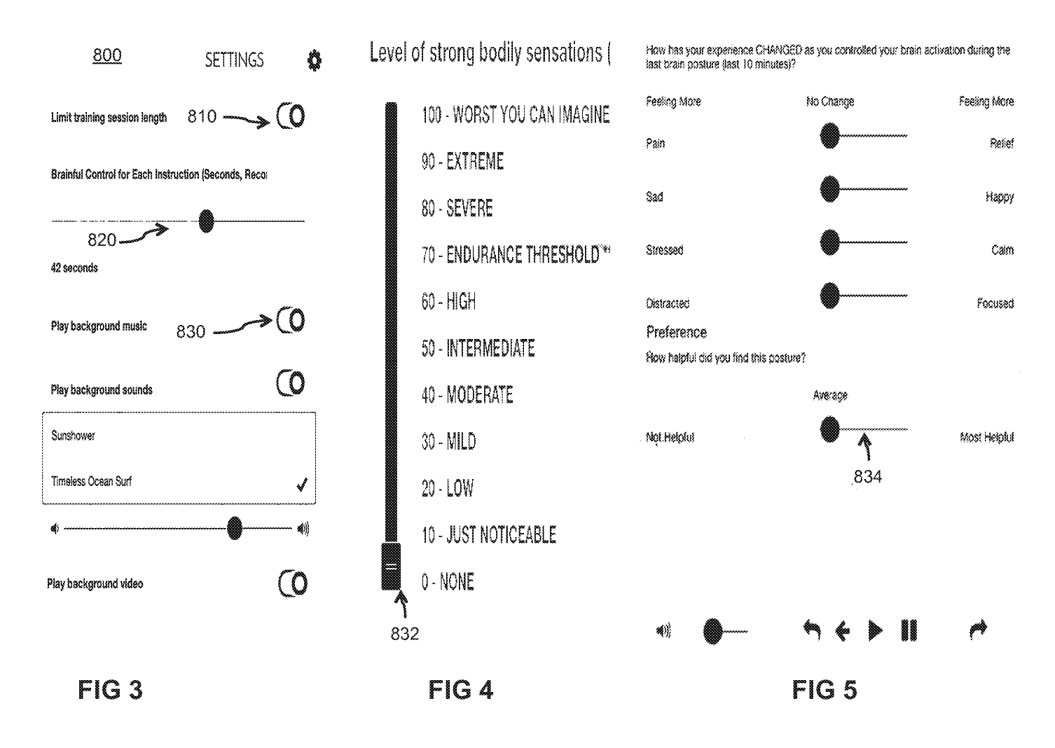

### FIG. 3. Example Settings Screen

A settings screen 800 may be provided to allow the user to adjust many features of the control software and what is presented to them. This may include a UI element to input the automatic limit to the length of each training session 810 before the control software indicates that the session is completed or takes the user to a final set of steps. It may include a UI indicator to select the length of time that the user will spend on each instruction or portion of in instruction, or on a delay period 820.

The selected delay period length may cause the software to ‘pause’ the program automatically for the user to complete their task, and/or provide a countdown of this duration 370 on the Training Screen. Software may cause UI indicators to allow the user to turn on/off or to select their choice of background music, background sounds, background images or video, and the volume, opacity or intensity of each 830. These settings/features may be saved from session to session by writing their values in a database for the user.

### FIG. 4. Example Mental State Input Screen

The software may provide a slider or other UI form element 832 that the user may use to indicate the level of a mental state. In this example, the user may indicate the level of the pain that they are experiencing.

### FIG. 5. Example Multiple State Input Screen

The software may provide a slider or other UI element 834 that the user may use to indicate the level of multiple mental states. In this example, the user may focus awareness on multiple mental/brain states and indicate the level that they are experiencing, such as pain vs. relief, sadness vs. happiness, stress or anxiety vs. calm, distraction vs. focus, and the helpfulness of an exercise vs. less helpfulness.

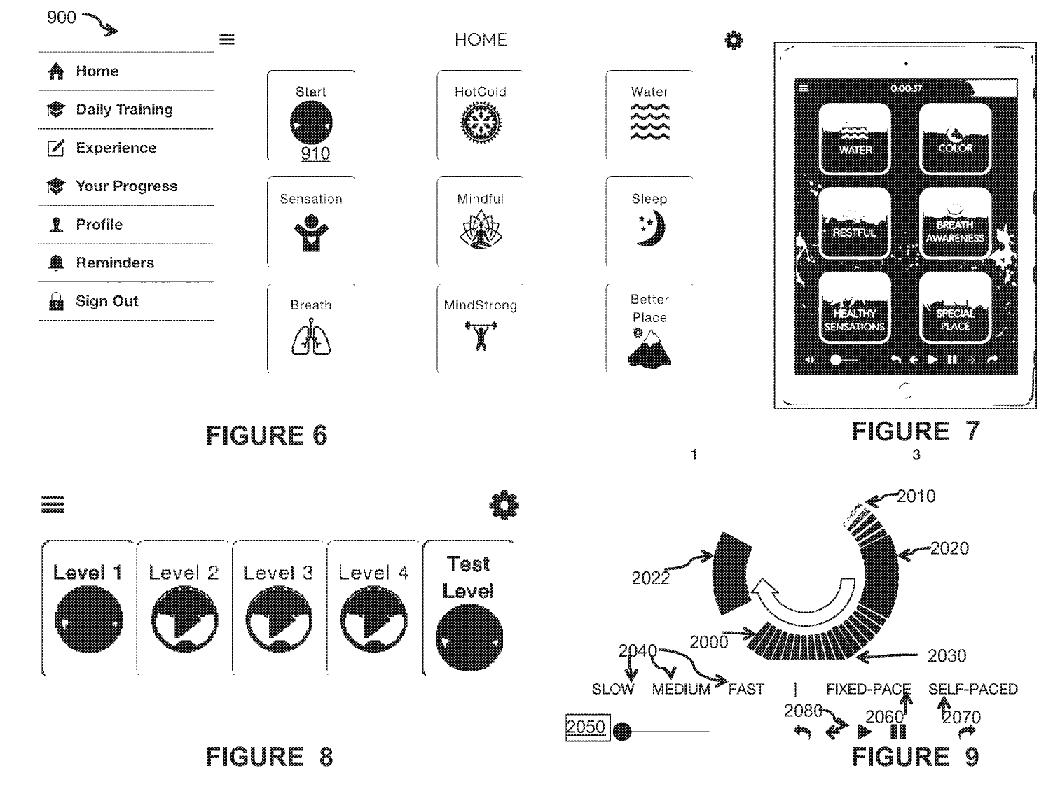

### FIG. 6. Example Slide Out Menu and Home Screen

The software UI may provide for a slide-out menu UI 900 that allows the user to select different features, including the ones shown, and to navigate to additional screens or content. The software may provide a home screen where the user may select different content, for example by selecting an icon 910 indicating the content being selected, for example with a name, level number, image, or an indication of whether or not the level is available, for example based on color or opacity. A screen may be provided that offers an app store or app marketplace like functionality, allowing a user to access or purchase content, or to receive more information or descriptions of the content, ratings from other users, or a trailer. The software may be provided via a variety of devices included a web browser or mobile device FIG. 7.

### FIG. 7. Example Home Screen on Mobile Device.

The software may provide a UI suitable for use on mobile devices such as tables, smartphones, wearables, watches and others. The UI may automatically adapt its content to fit mobile screen sizes, for example smartphones or tablets. The software may automatically rearrange and resize content to optimally fit any screen size, for example rearranging and resizing home screen icons to fit on a smartphone or tablet.

### FIG. 8. Example Level Selector Screen

The software may provide a level selector screen or UI element that may allow the user or guide to select the level of content that the user will receive. The levels may be indicated with a name, icon, color, opacity level or may indicate which level's are available based upon the user ‘unlocking’ levels through their performance, for example by locked levels being greyed out.

### FIG. 9. Example Pacing Screen

The software may provide a pacing screen that may guide the user through timed or paced stimuli, instructions, or exercises. In the example shown, which may be animated, the moving line 2000 may move around a circular path 2010. This may indicate the passage of time. The user may receive instructions to pace a task, for example a mental exercise, so that it has two (or more) phases. For example, the display may have two target regions 2020, 2022. The user may be instructed by the software to indicate when the moving line moves through one or the other of these target regions, for example by clicking a button or UI element, input into the software. The location of the moving line when the user clicks may be indicated by the software, for example by a differently colored or highlighted mark 2030, to indicate to the user how accurate their timing was. The user may receive stimuli or instructions that are aligned in time with the phase of the moving line moving around the circle. For example, when the line moves through the left target area 2022, the user may receive from the software one stimulus and/or be instructed to perform one task or mental exercise, or breathe in. When the line moves through the right target area 2020, the user may receive from the software a second stimulus and/or be instructed to perform a second task or mental exercise, or breathe out.

In this way, the pacing of the user's mental exercises may be indicated to the user, using a UI element with a repeating pattern that indicates time. The software may also receive input from the user that indicates when the user has completed each instruction, as indicated by their click. This may allow the software to determine the even rhythm of the user's performing a sequence of mental tasks, for example imagining a warm sensation while breathing in and clicking to the right, and imagining a cool sensation while breathing out and clicking to the left.

The pace of the rhythm and the display and accompanying audio may be controlled in a variety of ways. The pace may be selected by the software. The pace may be selected by the user's input, for example using UI elements such as buttons 2040 or a slider 2050. In addition, the software may select or allow the user to select fixed-paced 2060 or self-paced 2070 timing. In fixed paced timing, the software may use a constant time interval for each step or cycle, or for rotation of the circular screen element. The user may be scored by the software based on how closely their mental exercise performance as indicated by the timing of their clicks matches this fixed pacing. In self pacing, the user may click the interval after each step or mental exercise component, and the software may detect the timing between events, or the average timing between multiple events. The software may then set the pacing to equal the user's pacing input. The software may rotate the circular element, present any stimuli or instructions or any audio, in time coordination with this pacing. The software may score the user based upon the evenness of their performance of the task based upon the timing of their clicks. For example, users may be scored by the software based upon the percent difference in the current time interval between clicks vs. the previous interval, or the average interval. The screen may also include a controller 1080 to allow the user to select play, pause or to move forward or backward through any stimuli or instructions being presented, or to skip to the beginning or end.

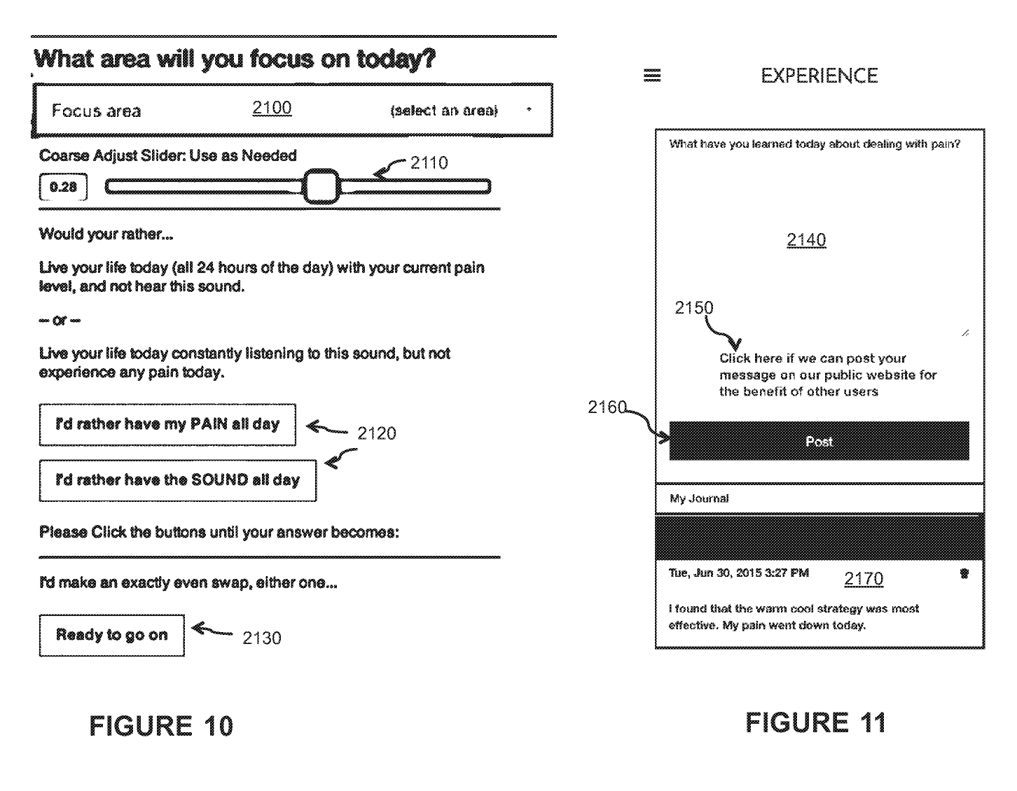

### FIG. 10. Example Paintone Screen

The UI may provide a screen and audio that the user may use to match the level of an internal state (such as pain level) to the unpleasantness or intensity of a sound. The software may provide a dropdown menu 2100, allowing the user to select an area of their body to focus on that has pain, from a list of different body areas. The software may provide a slider 2110 that controls the volume of an unpleasant sound that the user can use to indicate a match between the intensity or unpleasantness of their pain and the intensity or unpleasantness of the sound. For example, the volume of the unpleasant sound may be decreased by the software as the slider is moved to the left, indicating that the pain of the user has a low level of unpleasantness, and the volume of the unpleasant sound may be increased as the slider is moved to the right, indicating that the pain of the user has a high level of unpleasantness.

The software may provide a mechanism for the user to make a fine and exact measurement of the unpleasantness of their pain, by pressing buttons 2120 to make fine adjustments to the volume of an unpleasant sound so that it matches the unpleasantness of their pain. The software may use the selected volume from the coarse adjustment slider 2110 as a starting volume for the fine adjustments made by the buttons 2120. The software may require the user to choose which is more unpleasant between the sound and their pain, by pressing the appropriate button indicating either “I'd rather have my PAIN all day” or “I'd rather have the SOUND all day”. The software may update the sound based on the user's input. For example, if the user selects “I'd rather have my PAIN all day”, the software may marginally increase the volume of the sound to slightly increase its unpleasantness. If the user selects “I'd rather have the SOUND all day”, the software may marginally decrease the volume of the sound to slightly decrease its unpleasantness. The software may require the user to repeat this process until the unpleasantness of the sound exactly matches the unpleasantness of their pain. The software may provide a button 2130 that the user can press to indicate that the unpleasantness of the sound exactly matches the unpleasantness of their pain. The software may provide for paintone measurements of this type at other points in the stimulation, training, instructions, or exercises provided to users, for example to continuously measure the user's pain ratings during training or exercises or instructions.

### FIG. 11. Example Journal Screen

The UI may provide for the user to enter journal entries regarding their progress or notes. The software may provide a text box 2140 in which the user may enter journal entries about their pain experience. The software may allow input to the text box 2140 through a variety of input devices, such as a physical keyboard, digital keyboard, speech-to-text converter, or other appropriate device. The software may provide a button 2150 to allow the user to indicate that their journal entry may be shared publicly, and another button 2160 to submit the entry. The software may present previous journal entries to the user in a journal window 2170 that may show the date and time of the previous entry as well.

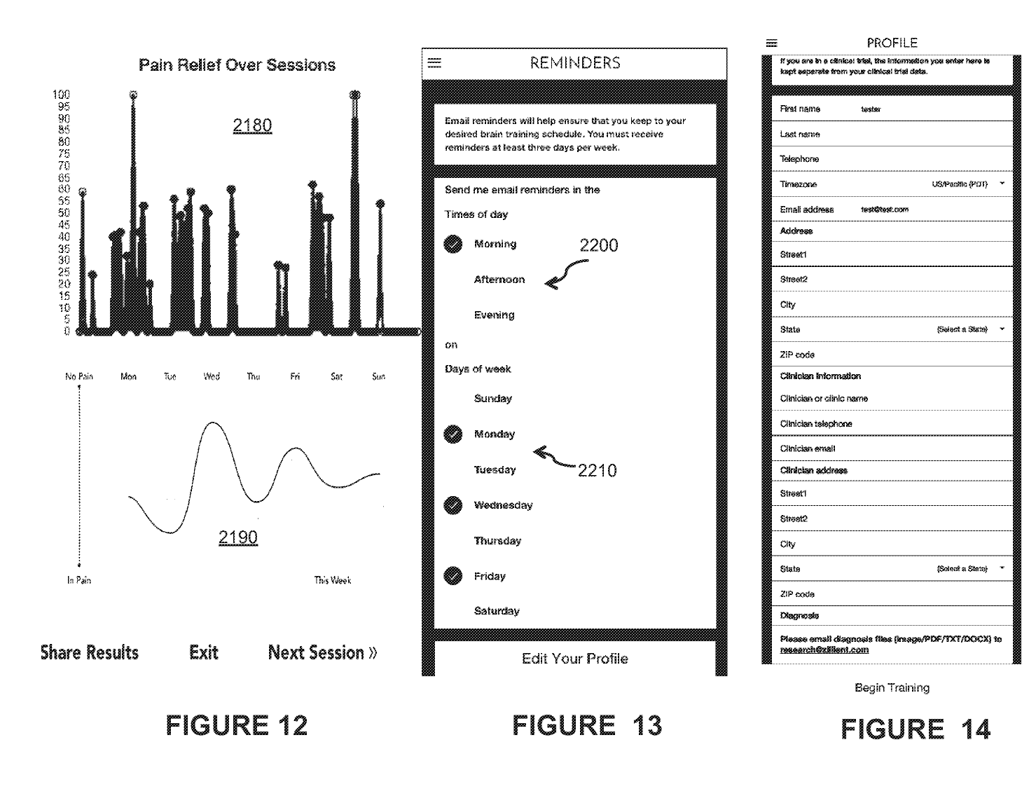

### FIG. 12. Example Progress and Statistics Screen

The UI may provide for graphs and other representations of the user's progress. The software may display a graph 2180 of user's history of software usage, for example showing number of minutes using the software on the y-axis and showing day number or session number or date on the x-axis. The software may also display a graph 2190 of the user's change in pain over time, for example showing the user's pain rating on the y-axis and the day number or session number or date on the x-axis.

### FIG. 13. Example Reminders Screen

The UI may provide for the user or guide/provider to select days or times when the software will send out reminders (email, text, phone, other) for the user to engage in training or remember to perform other tasks indicated by the software, or receive ‘micro-instructions’ such as short text or audio instructions individually selected for the user by the software, the user themselves, or the guide/provider. The UI may provide for the user to select the time of day 2200 and the day of the week 2210 to receive reminders.

### FIG. 14. Example Profile Screen

The software may provide a screen for the user to enter relevant personal information (including name, telephone number, email address, mailing address), to enter information about their treating clinician (including name, telephone number, email address, mailing address), and to upload a document (e.g. image, pdf, text files) verifying their clinical diagnosis of pain.

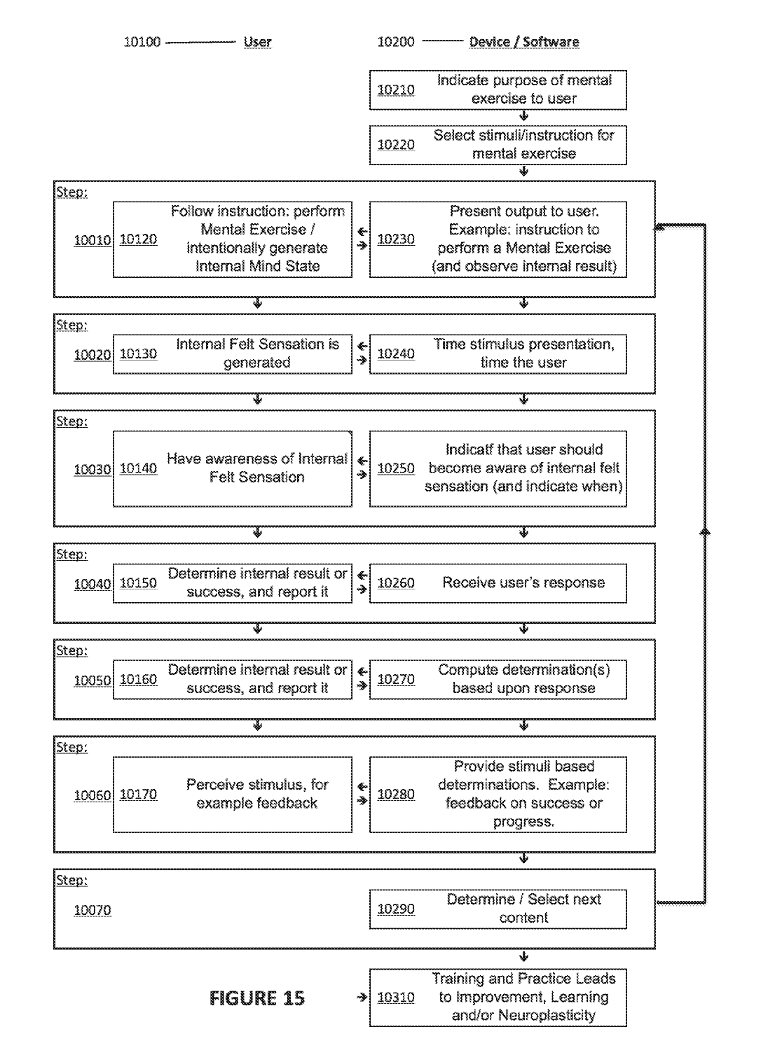

### FIG. 15 Example Basic Loop

The flow chart presented presents a basic example sequence of functioning of the methods, devices, software and systems provided herein. The device/software 10200 may function continuously as a loop based upon the input and responses of the user 10100.

Present Output to User

A variety of types of output or stimuli may be presented to the user. This output may guide the user through training or the performance of perceptions or exercises. This output may be timed, and may take a variety of different forms. Initial outputs may include indicating the purpose 10210 and selecting stimuli or instructions 10220, for example for a mental exercise for the user to perform. The output may be presented to the user 10230. The user may receive this stimulus or output or instruction. The user may attempt to follow this instruction 10120. The user may general an internal mind state 10120 based, in part, upon an instruction. This may lead to an internal felt sensation in the user 10130.

Timing

The software may monitor and control the timing of the presentation of output or stimuli or instructions and time the user's responses 10240. For example, the software may determine when to present each element of the output. The software may also determine for how long to present each element. In some examples, the duration of presentation by the software of each stimulus, content, or instruction may be about 600, 120, 30, 15, 10, 5, 4, 2, 1, 0.5, 0.2, 0.1, 0.01, 0.001, 0.000001 seconds. This determination may be based upon the input of the user, or attributes of this input. This determination may be based upon the input of prior users, or attributes of this input. The timing of output, stimuli, or instructions may be optimized by the software using prior information or data to improve the user experience, the desirability of the software, or the user's ability to effectively use the software. In particular, the timing may be based upon optimizing the time that the user interacts with each stimulus or instruction to improve their performance.

The software may also indicate or instruct that the user should become aware of or attend to the internal felt sensation or result of the stimuli, instructions or content that has been presented 10250. For example, if the software instructed that the user should perform a mental exercise (for example to generate the imagined feeling of warmth), the software may instruct the user to become aware of the result (such as the extent to which they feel warmth). The user may follow this instruction 10140, and generate a response indicating the result 10150, for example indicating what they are experiencing on a UI, which may be received by the software 10260. In some examples, the duration for which the user is instructed to become aware of or attend to the internal felt sensation may be about 1 second. In some examples, the duration for which the user is instructed to become aware of or attend to the internal felt sensation may be about 5 seconds. In some examples, the duration for which the user is instructed to become aware of or attend to the internal felt sensation may be about 15 seconds. In some examples, the duration for which the user is instructed to become aware of or attend to the internal felt sensation may be about 600, 120, 30, 15, 10, 5, 4, 2, 1, 0.5, 0.2, 0.1, 0.01, 0.001, 0.000001 seconds.

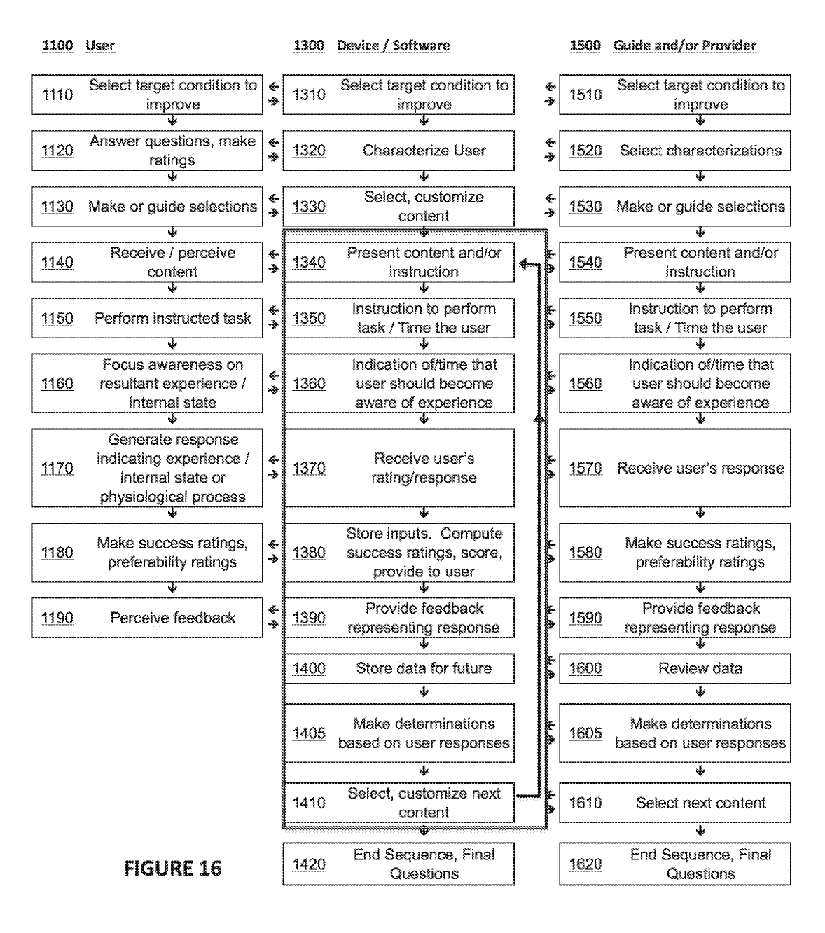

Timing-related steps may be provided by the software in substantially real time. Examples of timing steps that may be provided by the software in substantially real time include steps 10240, 10270, 10290, 1340, 1350, 1360, 1390, 1405, 1410. In some examples, the software may provide a continuous recurring loop for a period of time, as provided in FIG. 14, FIG. 15, FIG. 16. In order to complete the loop or repetitive loop in substantially real time, the software may complete individual steps in substantially real time. In some examples, the repetition time of the loop shown, such as the time between recurrences of each step, may be about 600, 120, 30, 15, 10, 5, 4, 2, 1, 0.5, 0.2, 0.1, 0.01, 0.001, 0.000001 seconds.

Substantially real time, as used herein, may refer to a short period of time delay, for example delay created by the software between stimulus elements, or between instructions or steps, or time delay between process steps, or the time delay between a user making an input and the software determining a response. Something may occur in substantially real time if it occurs within a time period of about 600, 120, 30, 15, 10, 5, 4, 2, 1, 0.5, 0.2, 0.1, 0.01, 0.001, 0.000001 seconds. The time increment selected may be based on what may produce an appropriate delay for a given situation, and/or produce a positive user experience or successful user training.

In some examples, the software may select or determine stimuli 10270, content, or instruction in substantially real time after the user has made an input 10260, so that the next stimulus, instruction, content or instruction may be provided by the software 10280 at an appropriate delay. In some examples it may be appropriate for this delay to be very short, for example less than one second or a fraction of a second. This may be appropriate for some examples where the software provides a precise timing exercise or game, or where the user is instructed by the software to perform instructions for a period of seconds.

In some examples, it may be appropriate for this delay to be of moderate length, for example 1, 2, 4, 8, 16, 32, 68, 128 seconds. This might be appropriate in examples where this delay provided by the software allows the user sufficient time to complete an instruction which requires a similar number of seconds, for example to perform a mental imagery or mental rehearsal task for 1, 2, 4, 8, 16, 32, 68, 128 seconds before receiving another instruction from the software.

In some examples, the software may select or determine next content 10290, including content, stimuli, or instruction in substantially real time after the user has made an input 10260, so that the next stimulus, instruction, content or instruction may be provided by the software 10280 at an appropriate delay. In some examples it may be appropriate for this delay to be very short, for example less than one second or a fraction of a second. This may be appropriate for some examples where the software provides a precise timing exercise or game, or where the user is instructed by the software to perform instructions for a period of seconds. In some examples, it may be appropriate for this delay to be of moderate length, for example 1, 2, 4, 8, 16, 32, 68, 128 seconds. This might be appropriate in examples where this delay provided by the software allows the user sufficient time to complete an instruction which requires a similar number of seconds, for example to perform a mental imagery or mental rehearsal task for 1, 2, 4, 8, 16, 32, 68, 128 seconds before receiving another instruction from the software.

Instructions

The user may be presented with instructions for a task to complete 10230. The instructions to perform tasks may take a variety of forms, including mental exercises.

Mental Exercises

The user may be presented with instructions to perform one or more mental exercises, or internal exercises.

An instruction to perform a covert, internal mental exercise may be different from an instruction to perform an overt, external exercise such as lifting a weight or assuming a body posture, or perceiving an external stimulus. An instruction to perform an internal mental exercise may be differentiable from an instruction to perform an outwardly-focused exercise in a number of ways. The differences between an internal and an external exercise may be understood by the user and may not be explicitly articulated in detail as part of the instructions. In general, the difference between an internal exercise and an external action or perception are broadly understood.

To ensure clarity, differences between an internal mental exercise that a user may be instructed to perform and an external or covert perception or exercise may include:

### 1) Internal Proximate Cause.

A mental exercise may have an internal, covert proximate cause. For example, an internal exercise may be caused by a decision of the individual to engage in it, which arises out of memory, decision-making, internal context or executive function of the individual. Externally-driven, covertly induced mental states may have an external proximate cause. For example, seeing a hot object may lead directly, and in a very short time, to the formation in the mind of an internal sensation of heat, the proximate cause being the peripheral sense organ of receptors in the skin. This is different from the formation of an internal mental sensation of warmth. An internal felt sense of warmth can include a mental image of warmth (though it can have a tactile quality or other qualities, not just a visual quality). In the case of the internal mental sensation, the sensation may be created from a combination of an intention that arises within the individual, and a memory of a past experience (e.g. a memory of a warm feeling).

### 2) Internal Proximate Result.

A mental exercise may have an internal, covert proximate result. For example, a mental exercise may lead primarily to changes in an internal felt perception or internal felt sense of an individual. This is different from the completion of an external (physical) exercise. If an individual completes a physical exercise, this primarily involves making physical movements with the musculoskeletal system of their body. These movements are the result of internal mental processes, however, they are tied to expressed physical movements as their primary expression. In contrast, if a person imagines making a movement, the primary expression of this activity is internal. As an example, practicing a tennis backswing is a typical external physical exercise, accompanied by physical movement, whereas practicing an imagined tennis backswing is a typical internal mental exercise, accompanied by an internal felt sense (sometimes called a mental image) of moving, but not primarily accompanied by the physical task. It should be noted that Internal mental exercises such as this can also be accompanied by lesser degrees of physical or musculoskeletal expression. For example, when someone practices giving a speech in their mind, while they may not actually speak, it is possible that their imagination will be partially expressed through concurrent lip or mouth movements. However, their primary intended result, and the primary observed outcome, is covert internal practice, not overt external expression.

### 3) Timing Relationship to External Events.

A primary differentiator between internal mental actions or exercises and externally driven actions or physical actions is their timing relative to external events. It can sometimes be difficult to make an absolute differentiation between internal and external events. Like warm and cold or bright and dark, they lie upon a continuum. For example, if one imagines the mental image of a remembered tree, this mental image may be created internally many seconds, minutes, hours, or even years after the event of having seen the actual tree that is being imagined. The timing delay between the actual external event (the eyes and sensory system focusing upon a tree) and the internal event (the forming of a mental image of a remembered tree), may be seconds, minutes, hours, days, even years. If one sees an actual tree, then the sensation or perception that arises in the mind as a direct result typically takes place within a period of around a second or a fraction of a second: one nearly immediately sees a tree. The neurophysiological signal arising in the peripheral receptors of the retina lead to a brain representation of a tree within a few hundred milliseconds or even less.

Similarly, if one engages in the internal mental exercise of imagining performing a physical act (one practices internally or forms and adjusts a motor plan), one might not engage in an outward physical movement act for minutes, days, weeks, or even years later if ever. If one engages in performing a physical act, the relative time delay between the mental and neurophysiological signal to move or act and the actual movement or action is typically in the range of a second or a fraction of a second.

Also, an internal mental exercises
4) Capable of being Internal Only.

An internal mental exercise or action is one that is capable of remaining wholly internal or covert, whereas an externally-driven perception or action normally is not. For example, an individual may intentionally choose to imagine making a movement, but withhold actually making the movement, so that it remains internal. A physical movement is actually expressed through the movement of the body in the world. An individual may be capable of forming a mental image of a tree with no physical external tree present, and they may through the methods, devices, software and systems provided herein learn to improve their ability to form a mental image. An individual is not normally capable of creating the experience of perceiving an actual tree in the absence of the existence and sensation of the external object.

### 5) Generality.

Internally generated objects and mental exercises are typically more general and less detailed and specific that externally-driven or externally-expressed objects and exercises. For example, one can imagine the general idea of a tree, which is less specific than the perception of an actual tree. One can imagine the general idea of taking a physical posture, without specifying one's exact body shape. The current methods, devices, software and systems provided herein are also capable of training individuals to become more specific in the internally generated objects and exercises that they are capable of producing.

Visual

The user may be presented with a stimulus or an instruction to perform a visualization. For example, the user may be instructed to visualize warm water flowing over a part of their body where they are experiencing pain. Many additional types of ‘visualization’ are indicated below. While the word ‘visualization’ may connote a visual image, the user may be instructed and may intend to perform exercises guided toward activating or imagining any type of mental construct in any cognitive, sensory, motor, emotional or other mental domain.

Stimuli designed to guide the user in a visual mental image task or visualization may include images (for example the image of a color, the image of a body part to attend to, the image of something to imagine, the image of a place to imagine or imagine being, the image of a person to imagine being or imagine being with); video (for example a video of: a scene to imagine, a scene to imagine being in or imagine one's reactions in, a person one is with, a person whom one may imagine being, an object, a body movement to imagine, a body movement to perform, a breathing pattern to mimic), audio, or verbal or written instructions to perform similar visual mental tasks.

Tactile

The user may be presented with a stimulus or an instruction to create a mental tactile experience. For example, the user may be instructed to intentionally create the tactile feeling of warm water flowing over a part of their body where they are experiencing pain. Stimuli designed to guide the user in a mental tactile task may include images (for example the image of a body part to attend to, the image of something to imagine, the image of a place to imagine or imagine being); video (for example a video of: an object the user can imagine being in contact with, a body movement to imagine, a body movement to perform, a breathing pattern to mimic), audio, or verbal or written instructions to perform similar visual mental tasks. A user may be presented with tactile patterns to discriminate, remember, remember the sequence of, or to imagine new patterns or sequences or combinations of.

Auditory

The user may be presented with a stimulus or an instruction to create a mental auditory experience. For example, the user may be instructed to intentionally create the sound of water flowing over a part of their body where they are experiencing pain.

Stimuli designed to guide the user in a mental auditory task may include images (for example, the image of something to imagine, or the image of something that makes sounds that the user can imagine, the image of a place to imagine the sounds from or imagine being, the image of a person to imagine being or imagine being with); video (for example a video of: a scene to imagine, a scene or sounds from a scene to imagine being in or imagine one's reactions in, a person one is with, a person whom one may imagine being, an object, a breathing pattern to mimic), audio, or verbal or written instructions to perform similar visual mental tasks. In the case of audio, a user may be presented with audio to then remember and later form a mental image of or practice. A user may be provided with music or musical sounds or pleasant or unpleasant sounds to listen to during practice of mental exercises or to remember and create a mental experience of. A user may be presented with auditory patterns to discriminate, remember, remember the sequence of, or to imagine new patterns or sequences or combinations of. An example of a verbal instruction is that the user may be instructed to mentally imagine things that the user has gratitude for, or write a list of things that the user has gratitude for.

Motor

The user may be presented with a stimulus or an instruction to generate an external movement, or an internal, mental performance of a motor task, such as imagining performing a movement or sequence of movement. For example, the user may be instructed to imagine performing a jumping jack exercise.

Stimuli designed to guide the user in a mental motor task or visualization may include images (for example the image of a body posture, the image of a body part to imagine moving, the image of something to imagine moving, the image of a place to imagine or imagine being); video (for example a video of: a person performing a movement or movement sequence or dance or athletic sequence breathing sequence or yoga sequence), audio, or verbal or written instructions to perform similar visual mental tasks. The software may also present the user with a representation of the user performing a motor task or imagined motor task.

Emotional

The user may be presented with a stimulus or an instruction to generate an emotion, or an emotional response, or to suppress or avoid an emotion or emotional response, or to replace one emotion with another one. For example, the user may be instructed to imagine being afraid, thereby evoking the feeling of fear. The user may be provided with may types of stimuli to aid in evoking this emotion, such as objects, people, or situations that the user may be afraid of. These may be presented using any modality of stimulation.

Stimuli designed to guide the user in generating or avoiding an emotion may include images (for example the image or video of an object that generates or alleviates the emotion, the image or video of someone helpful in dealing with the emotion, the image or video of a place to imagine or imagine being the evokes an emotion), audio, or verbal or written instructions to perform emotional tasks.

Example emotions and stimuli that the software may present so that the user may use them to evoke emotions include: Fear: combat, pain, ill-health, loss, physical inability, heights, animals/snakes, social situations, violence, loss of money or an object; Anxiety: stressful situations or memories; Depression: sad people or faces or situations; Craving: stimuli that induce craving such as food, alcohol, drugs or illicit substances; self-described situations that evoke or sooth an emotion.

Craving/Satiety/Gustatory/Addiction—Related

The user may be presented with a stimulus or an instruction to generate or inhibit/prevent a sense of craving, satiety, or a taste or gustatory sense or the sense of eating something, or the craving for, satiety from, or use of addiction-related stimuli. Addictions and addiction-related stimuli include alcohol, substances including illegal drugs such as narcotics, any of the drugs mentioned elsewhere in this document, stimulants or depressants, gambling, sex/love, pornography, internet, gaming, smoking, nicotine, food, video games, shopping, work.

The software may provide users with stimuli meant to evoke craving for or the sensation of receiving or the sensation of satiety from any of these or other elements. The software may provide users with stimuli meant to evoke withhold or withdrawing any of these or other elements.

Stimuli designed to guide the user in generating or avoiding craving may include images (for example the image or video of an object that generates or alleviates the craving such as cigarettes, drugs, sex, games, food, drink, alcohol, shopping, goods, or the consequences of engaging with any of these, or the situations or people associated with engaging with any of these, the image or video of a place to imagine or imagine being that evokes the sensation), audio, or verbal or written instructions to perform or avoid these tasks or perform or avoid these imagined tasks.

Memory-Related

The user may be presented with a stimulus or an instruction to generate or inhibit/prevent a memory of a past experience that they have had. Memories may include traumatic memories, memories of a loss or lost person, memories of something that induces sadness, or memories of a place, time or person with positive associations. The software may collect input from the user regarding such memories, including recorded audio, speech, text, images, video or other input. This information may then be used as stimuli to present back to the user to induce or inhibit such memories, or as a part of training.

Plan-Related

The user may be presented with a stimulus or an instruction to generate or focus on a plan for the future, or to visualize, generate, or refine thins plan in their mind, or to perform written or other exercises to sharpen the plan. Plans may include plans for overcoming challenges such as addiction or depression or anxiety or pain. Plans may include elements of life-planning such as financial planning, envisioning or describing a positive relationship or relationship characteristics, educational or career plans, or other elements of future planning that a user may want to engage in. The software may collect input from the user regarding such plans or positive visions or vision boards or vision statements, including recorded audio, speech, text, images, video or other input. This information may then be used as stimuli to present back to the user to induce mental imagery or thoughts or exercises related to such plans, or as a part of training.

User-Created

The user may create content for use by the software in their own training, or in the training of other individuals. For example, users may record audio or text instructions for use by the software, or upload content including audio, images, video, text. An administrative interface may allow users to record or to upload recordings or images or video or other types of files or content or text to be used as stimuli. See User-Created Content Offerings for further information.

Meditation

The user may be presented with meditation instructions. These instructions may include any instructions known to be a part of meditation practices. Examples include instructions in breathing, deep breathing, relaxation breathing, breath awareness, body scanning, Vipassana, Zen, Tong Lin, TM, centering, visualization, mantra, tantric practices, lucid dreaming, yogic breathing, yogic practices, relaxation.

Paired Exercises, Sequenced Exercises

Exercises presented may be presented in pairs, or in sequences. For example, a user may alternately be instructed to imagine a warm sensation and then a cool sensation, and then repeat. This may also encompass longer sequences. Instructions may be provided before and/or after individual sequence elements, or instructions may be provided prior to or after the time that the user practices the entire sequence. Sequences of exercises may be stored, and presented to users. Sequences of exercises may also be generated algorithmically.

In paired exercises, the two elements of the pair may be opposites. The two elements may complement each other. The user may use their breath to match the timing of paired exercises.

The software may provide a pre-created sequences of exercises. This sequence may be selected to be beneficial in a number of ways, including using warm-up/intro or cool-down/outro exercises, or using exercises that follow a logical sequence that builds up a skill, or that support one another. An example sequence is a sequence of physical postures used in yoga or in a stretching routine. Another example sequence is a sequence of imagined physical postures similar to those used in yoga or in a stretching routine, but based on mental generation by the user.

Groupings of Stimuli and Exercises

The software may provide groupings of stimuli. For example, a screen may provide exercises designed to be helpful for particular goals, for example pain, depression, anxiety, stress, sleep, meditation, relaxation, focus, concentration, learning, memory, or others. Within one of these goals, there may be multiple exercises. For example, for the goal of helping pain, there may be a screen with multiple exercise sequences. If a user selects one of these exercise sequences, then the software may present a sequence of different stimuli or instructions. The sequence of these stimuli or instructions may be stored, or may be created in real time. Within the sequence, stimuli or instruction steps may be provided individually, in pairs, or in sub-sequences. There also may be variants of each step. For example, one step may be to imagine increasing the temperature of an area where someone is experiencing pain. Another step may be to imagine decreasing the temperature of an area where someone is experiencing pain. The user may receive instructions to alternate back and forth between these two steps. In addition, at successive times, the user may receive alternate variants of these instructions. For example, the user may receive the instruction to imagine warm water in one cycle, and may receive the instruction to imagine a warm stone in another cycle. The variants may also constitute levels of varying difficulty. For example, the user may first be instructed to complete an easy variant, and once this has been completed, the user may later be allowed or instructed to complete a more difficult variant. The level of difficulty may be determined by the success of the user on previous trials, or the success of previous users.

The software may allow the user to ‘unlock’ steps, sequences, levels, or exercises. The software may allow the user to unlock them based on their performance, for example the user may be required to accomplish a goal or reach an adequate score on one level before the next level is unlocked, or is no longer greyed-out on a screen. The software may also allow the user to unlock goals, exercises, levels or other content through signing up for or purchasing a subscription. Subscriptions may also be time-limited. The software may provide a Freemium trial period for the user to try content before signing up for a subscription, or paying for a subscription. The software may provide for an affiliate program if users encourage others to participate or to subscribe.

Games

The software provided may included elements of a game, for example a computer game. Such elements include motivations for the user, scoring, reward screens with sounds, animations, video or even monetary rewards. All of these elements may be used to motivate users and to make the use of the software or training more enjoyable. For example, if a user is undergoing training, the software may provide different game worlds, different levels, may score the user, may provide animations, or sounds, and may provide other elements familiar to computer games, educational games, or neurogaming.

Physical Exercises

Users may be presented with physical exercises or with perceptions following any or all of the same processes described above for mental exercises, only with the difference that the user actually performs an overt physical task, or experiences an overt physical stimulus, or both. This may be performed in combination with mental exercises. This may also be performed as an alternative to mental exercises. Mental exercises may also be proved as an alternative to physical exercises, for example in individuals who are not capable of performing physical exercises or who do not want to. For example, someone who is injured or otherwise impaired may be able to practice a mental exercise in place of a corresponding physical exercise that they are not capable of performing, or choose not to perform. Over time, it is possible that this will enable them to progress to the point that they are capable of or choose to participate in the physical exercise. This process may have application in rehabilitation, physical therapy. An instruction to engage in a physical exercise may be presented so that a user may understand the exercise, and the user may at a later time practice a corresponding mental exercise. For example, if a user in instructed to open and close their hand and they perform this physical exercise, they may later be instructed to imagine opening and closing their hand. Performing the physical exercise may be beneficial to a later performing imagined exercise, and performing a mental exercise may be beneficial to later performing a physical exercise.

Athletic Training Sequences/Yoga

Users may be trained in performing sequences of physical exercises. These sequences may include athletic training sequences, stretching sequences, dance sequences, or yoga posture sequences. These sequences may be pre-stored, and may be customized and selected for individual users.

The software may be provide to train individuals in yoga sequences, either using actual physical movements, or imagined movements. For example, individuals may be led through the Ashtanga series, or other sequences that have been or may be developed, for example Vinyassa Flow or others.

For each posture, the individual may receive instruction suitable to the individual's level. For example, a beginner may receive easier variants of postures than an expert. An individual may select for each posture or exercise which variant is suited to them. This information may be stored so that a user may customize the sequence instruction that they receive. The user may also customize the time that they spend on each sequence element or posture. For example, the user may select an overall timing constant which is multiplied by the stored time to be spent on each sequence instruction element in a sequence, or each posture. Alternatively, the user may select a time for each sequence element or posture individually. These values may be stored for future use. These instructions may be provided by audio, for example using headphones and a mobile device, so that the user may receive instructions for performing a yoga or athletic training sequence while they are performing it. These instructions may be further tailored in real time, based on the user indicating when they have completed each sequence step, or selecting an overall timing pace or difficulty level for the day, or overall training duration for the session or the day. The features described here for sequencing, customization, timing and personalization for yoga sequences or athletic sequences, real or imagined, may also be applied to other types of training or to other types of mental exercise training provided by the software.

Tactile

Stimuli presented to users may include tactile stimuli, including taps, or vibrations, or pulsations, or a tactile rhythm, or warm or cold stimuli. These stimuli may be presented in combination with any aspect of the methods, devices, software and systems provided herein described. In one example, a tactile stimulus may be presented to a user to focus the user's attention on a body part where the user is attempting to focus attention, such as an area where the user is experiencing pain. A tactile stimulus may be used for sensory discrimination training. A tactile stimulus may be used as a sensory replacement for other sensations that a user is experiencing, such as pain. Through focusing attention on this tactile stimulus, a user may learn to replace an undesirable sensation such as pain with a more desirable one, such as the tactile stimulus. A tactile stimulus may be used to give a user something to focus attention on in an area of their body. The magnitude of the tactile stimulus may be changed or decreased. This decrease may be made using adaptive tracking or other methods so that as the user becomes better at detecting or focusing on or making sensory discriminations of the tactile stimulus, the stimulus intensity or differentiability may be decreased, maintaining a challenge for the user. The repeated presentation of a tactile stimulus may produce neuroplasticity. The tactile stimulus may be provided by a mobile device. For example, the tactile stimulus may be made by the vibration of a smartphone. For example, the user may place a smartphone on a part of their body where they are experiencing pain in order to perceive the tactile stimulus of the device vibrating. The software may control the device to vibrate following timing patterns or intensity patterns that the user may be instructed to attend to, or make determinations about, or make discriminations among. For example, the user make be instructed to determine which of more than one tactile stimuli is longer or shorter, stronger or weaker, to discriminate vibration frequency, or to count the number of tactile stimulus events, or to detect a tactile stimulus that is different from others.

Timing Information

The software may monitor and control the timing of the presentation of output or stimuli or instructions and time the user's responses 10240. For example, the software may determine when to present each element of the output. The software may also determine for how long to present each element. This determination may be based upon the input of the user, or attributes of this input. This determination may be based upon the input of prior users, or attributes of this input. The timing of output, stimuli, or instructions may be optimized by the software using prior information or data to improve the user experience, the desirability of the software, or the user's ability to effectively use the software. In particular, the timing may be based upon optimizing the time that the user interacts with each stimulus or instruction to improve their performance.

Visual

Visual timing information provided by the software may include a moving object that moves with a fixed timing. For example, the software may provide a moving object that moves in a circle, like a clock, that moves back and forth, that moves like a metronome, that moves like a pendulum, that moves in and out, that gets smaller and larger, that changes color. Any of these elements may be used to indicate the passage of time.

In the case where the visual information is presented as a rotating line 2000, as in FIG. 9, the software may also present a visually-presented target zone, which indicates the zone of a response with correct timing. The software may also present accuracy feedback, such as a marker indicating the position of the line at the time when a user made a selection 2030.

Audio

Stimuli presented to users may include written text, text to speech, spoken text, music, sound effects, sound icons, or others. In the case where stimuli are presented in pairs, or in sequences, one sound may be used to represent each element in an alternating pair, or in a sequence. For example, in alternating between two instructed elements, a user may be presented with one sound for/during one element, and a second sound for the other/during the other element. This may provide a way for the software to present the rhythm/timing to the user.

Scoring

Information relating to a score determined for the user by the software may be provided to the user in a number of ways. Information on the user's score may be provided by the software numerically, for example by providing a points tally, in real-time as the user collects the points, or after an exercise showing the points-total during an exercise, or in high points or comparison to other users lists, or in any other configuration and at any other time. The score may also be represented by the software numerically as a number of hits (number of user inputs that are correct) and misses (number of user inputs that are incorrect), either in real-time as the user makes inputs, or in a summary screen at the end of an exercise, or in any other configuration or time. The score may also be represented by the software by providing icons or graphic images indicating their score, for example images or animations of ‘coins’ or ‘badges’ awarded to users when they make a correct response, or using graphics representing changes to brain activation patterns, or filling in brain areas or emptying brain areas, or changing their colors, or showing connections or changes in connections between brain areas or neurons. The user's success may also be indicated by sounds, such as a sound that is presented for ‘hits’ and a different sound for ‘misses’, or a sound whose parameters (such as pitch, volume, duration, selected sound file contents) are based on the user's timing accuracy.

The user's score may be provided back to the user at a number of times. The score may be presented to the user in real-time while the user is interacting with the software exercises. For example, a points-tally that is continuously updated based on the user's inputs may be visually presented to the user on the screen while they are interacting with the software. The score may also be presented to the user at any time that the user is not interacting with the software exercises. For example, the score may be presented to the user in a post-exercise summary screen, in a leaderboard, in a list of the user's high scores, by email or message, and so on.

A target score may be presented to the user. The target score may be any score that the user is asked to achieve by the software. For example, the target score might be a target level of pain reduction, a target level of software usage, a target within-exercise accuracy, and so on. For example, at the beginning of an exercise the user may be asked to achieve 5 correct responses (hits) during the upcoming exercise, or the user may be asked to try to achieve a 20% reduction in their pain over the period of the upcoming. The target score may be presented at any time to the user, including but not limited to the user's first interaction with the software, or at the beginning of each software session, or at the beginning of each exercise. The software may allow the user to ‘unlock’ steps, sequences, levels, or exercises based on the target score.

The user's score may be stored, summed across trials and/or sessions, compared with scores from other users, and in other respects used to make the process more enjoyable, challenging, and motivating.

Leaderboard/Teams

The software may provide a leaderboard, or other means for comparing and displaying the scores or progress of users. This may allow a user to compare their performance with others. The software may provide a means for users to select teammates, or to have teammates selected for them. Then, the progress, score, or accomplishments of a team may be monitored, and/or compared with other teams.

Tracking Progress Over Time

The progress of a user may be tracked and presented. This may include elements of the users performance, such as how much they have trained, or how much time they have trained for, or how many exercises they have completed. This may also include elements of the user's symptoms, such as the users pain, depression, anxiety or other symptoms, or their ability to control these symptoms. These values may be plotted over time to demonstrate progress. These values may be presented in a calendar format to indicate daily actions or progress.

Measurement UI Elements

The software may provide UI elements to allow a user to enter their experience or progress. These elements may include sliders, drop down menus, selectors, buttons, or other UI form elements. The user may use one or more of these inputs to rate aspects of their experience. Some of these aspects may include their pain level relief, sadness or happiness, anxiety or calm, focus or distraction, craving or satiety or other indicators of their experience. These UI elements may also measure the user's assessment of their progress or success, for example their success in completing an exercise or instruction.

Text/Audio/Images/Video/Location

Stimuli presented to users may include written text, text to speech, spoken text, music, sound effects, sound icons, or others. Stimuli presented to users may include images, animations, or video.

These stimuli may be intended to represent a real or imagined action that a user may take. For example, a user may be presented with a variety of stimuli that indicate that the user should mentally generate the experience of opening and closing his/her hand. Stimuli that could connote this to the user include text descriptions of this, verbal descriptions, images of a hand opening and closing, an animated hand, or a video of a hand opening and closing. Audio stimuli may also include binaural cues, binaural beats, shepherd tones, the McGurk effect and other auditory illusions. Visual stimuli may be provided the induce visual illusions. Illusions may be provided as a means of indicating to subjects the possibility of changing perceptions, or of sensory plasticity or learning.

The software may be provide to associate stimuli or instructions with locations or trajectories in space. Audio stimuli may be presented using stereo information or other auditory cues to simulate position or movement through space. For example, in pairs or sequences of stimuli, each stimulus or instruction may be associated with one location or trajectory through auditory space, for example the trajectory from left ear to right ear. Visual stimuli may be presented using location information so that each exercise, or sequence, or step, is associated with a location or trajectory in visual geometric space, color, or movement.

Make Determinations Based Upon User Input 10270

The present methods, devices, software and systems provided herein may make any or all of a variety of types of determinations based upon a user's input. These determinations may be used to guide the ongoing progress of the user's training. This may be used to create a continuous improvement and learning process of the user. The user may also use the results of these determinations to provide motivation. The results of these determinations may also be used to help a guide or professional to evaluate the user, their progress, or to select future actions for the user.

Stimulus Selection

The user's input may be used by the software to guide selection of stimuli, content, or instructions for presentation to the user 10280. In the case where the software is presenting the user with feedback, for example feedback regarding the user's progress or performance, the user may perceive this stimulus, instruction or information 10170.

Based on User Ratings

The user may make inputs that may serve as ratings of portions of the output, stimuli, or instructions that the user has received. The user may make inputs that may serve as ratings of the experiences that the user has had as a result of the output, stimuli, or instructions that the user has received. For example, a user may rate a stimulus using a binary rating, such as thumbs up or thumbs down. A user may rate a stimulus using a Likert scale, slider, or other UI element to indicate the level of their rating. A user may rate a stimulus using qualitative, written, or spoken input.

The software and system may make determinations based upon these ratings regarding what output, stimuli or instructions to present to this user or other future users at the present time or at a later time. For example, the software may create a rating measure for each stimulus component based on one or more of the user's ratings of this stimulus component, or other user's ratings of this stimulus component. This rating may be used to determine the timing, frequency, or probability of presenting this output stimulus or instruction to the user, or to future users. Stimuli that have been more highly rated may be presented with higher probability. An algorithm may be provided that seeks to balance collecting input regarding stimuli to assess an accurate determination of reactions to these stimuli and receive resultant ratings, while also attempting to present stimuli which have higher ratings.

Based on User Success

The user may make inputs that may serve as ratings of their success in completing certain instructions or mental exercises or having certain mental experiences or an indicated internal felt sense in response to portions of the output, stimuli, or instructions that the user has received. The user may make inputs that may serve as ratings of the success that the user has had as a result of the output, stimuli, or instructions that the user has received. For example, a user may rate their success in using a stimulus using a binary rating, such as thumbs up or thumbs down. A user may rate their success in using a stimulus using a Likert scale, slider, or other UI element to indicate the level of their rating. A user may rate a stimulus using qualitative, written, or spoken input.

The software and system may make determinations based upon these ratings regarding what output, stimuli or instructions to present to this user or other future users at the present time or at a later time. For example, the software may create a rating measure for each stimulus component based on one or more of the user's ratings of their success using this stimulus component, or other user's ratings of their success in using this stimulus component. This success rating may be used to determine the timing, frequency, or probability of presenting this output stimulus or instruction to the user, or to future users. Stimuli that have been more highly rated may be presented with higher probability. An algorithm may be provided that seeks to balance collecting input regarding stimuli to assess an accurate determination of success in using these stimuli and receive resultant ratings, while also attempting to present stimuli which have higher success ratings.

Representing their Response

The user may make inputs that may serve as indications of their qualitative or quantitative response to a stimulus or instruction or the action that they took as a result. For example, a user may select a position along a left-right or up-down continuum on a user interface to indicate the level of a sensation that they are experiencing, the level of sensation that resulted from a stimulus, or the level of sensation resulting from the mental or other action that they performed in response to a stimulus or instruction. For example, if the user receives an instruction for a mental exercise intended to decrease pain, the user may make an input representing the level of pain that they experienced during or after the action more mental task that they undertook as a result of this instruction.

Real Time

Output being presented to users may be updated in substantially real time based upon user input. For example, the user's input may lead to a substantially immediate change in sound level, sound selection, sound quality or parameters, image selection, image opacity, image brightness, image timing or rhythm.

Stimuli Representing to Input

Stimuli that are altered in real time may be intended to represent the input being provided by the user, for example representing intensity, quality, or quantity. For example, if a user selects a position along a left-right or up-down continuum on a user interface to indicate the level of pain sensation that they are experiencing, the software may determine a corresponding sound volume or sound pitch to present to the user, and may update the sound presented to the user in substantially real time. If a sound is intended to represent pain, it may be made louder in correspondence with the user's input. If a user selects a position along a left-right or up-down continuum on a user interface to indicate the level of pain sensation that they are experiencing, the software may determine a corresponding image or video intensity or opacity to present to the user, and may update the stimulus presented to the user in substantially real time. If a visual stimulus like an object, image or video is intended to represent pain, it may be made louder in correspondence with the user's input. If a user selects a position along a left-right or up-down continuum on a user interface to indicate their level of success in completing an exercise that they are experiencing, the software may select stimuli, instructions, words, images, sounds to immediately present to the user. This same process may be used for continua input by the user other than pain or success, including other types of input described for the methods, devices, software and systems provided herein.

Perceptual Ratings/Progress

The software may provide an input for the user to make ratings of their perceptions. From this information, the software may make determinations of the user's progress. The user may also make ratings of their progress. For example, the software may allow the user to rate changes in their symptoms that they are trying to alleviate (for example, pain, depression, anxiety, stress, craving), or to rate changes in desirable aspects of their experience (for example focus, calm, relief, satiety). The software may provide for the user to make perceptual ratings of an internal experience that they may have generated or internal mental exercise that they may have performed. For example, if the user is instructed by the software to imagine creating warmth or coolness, the software may provide for the user to rate the level of warmth or coolness that they were able to create. If the software instructed the user to imagine lifting weights in their mind, the software may provide for the user to rate how much weight they lifted, how many times, when they started or stopped, or their feeling of exertion, exhaustion, mental fatigue or other aspects of their experience. If the software instructed the user to decrease pain in their mind, the software may provide for the user to rate how much pain they experience, how intense, over what physical extent, and/or with which qualities, or other aspects of their experience.

Timing

The software may control the timing of the presentation of output or stimuli or instructions. For example, the software may determine when to present each element of the output. The software may also determine for how long to present each element. This determination may be based upon the input of the user, or attributes of this input. This determination may be based upon the input of prior users, or attributes of this input. The timing of output, stimuli, or instructions may be optimized by the software using prior information or data to improve the user experience, the desirability of the software, or the user's ability to effectively use the software. In particular, the timing may be based upon optimizing the time that the user interacts with each stimulus or instruction to improve their performance.

The software may use the user's input to determine the user's score. The score may be determine by the software based on the user's ability to do the task. This may include how accurate are the user's timing of responses. For example, if the user attempts to press a button at a specific software-determined time on each cycle (e.g. 4 seconds), the score may be based on the numerical difference between the target time and the time of the user's input (e.g. 4 minus 3.7 seconds). The score based on the user's ability to do the task may also include the total number of correct versus incorrect instances of the user's input. For example, if the user attempts to press a button at a specific software-determined time on each cycle (e.g. 4 seconds), the score may be determined using the total number of times that the user pressed the button within a given window of that timing (e.g. within +/−30% of 4 seconds), summed over the entire period of the exercise. In this example, the score would be expressed as a number of ‘hits’; i.e. the number of times that the user correctly gave an input at the correct timing.

The score may also be determined by the software on user's ratings of their internal state. This may be in the form of a continuum. For example, if the user is asked by the software to rate their degree of success in visualizing a mental state or performing a mental exercise on a scale from 0 to 10, the score may be based on the number on the scale that the user selects on a software UI. Another instance of this may be based on a binary choice; for example, the score may be a ‘1’ or a ‘0’ based on if the user reported that an exercise “worked for them” or “did not work for them”. The score may also be based on defined ratings selected and input into the software by the user, such as low, medium, high. The score may also be based by the software on any other user input regarding their internal state.

The score may also be determined by the software based on other measurements that the software makes of the user. The software may include input information about the user's usage of the software in this determination. For example, the score may be based on how often (e.g. how many days in a given month) or how long (e.g. number of minutes per day) the user uses the software or performs mental exercises.

The user may receive separate scores for any of the different assessments that they input, or for combinations. For example, the user may receive a score for the duration of their mental exercise, times the accuracy, times their perception of their success, each weighted by an appropriate factor.

Sequence Order

Content, stimuli, instructions or exercises may be presented by the software to the user in sequences. Sequencing may occur globally for different types of exercises. For example, the software may determine that on day 1 the user interacts with an exercise type that trains the user how to use the software, then on day 2 the user interacts with an exercise type in which the user does switching between hot/cool mental states, and then on day 3 the user interacts with a breathing exercise type. The software may provide a pre-created sequences of exercises. This sequence may be selected to be beneficial in a number of ways, including using warm-up/intro or cool-down/outro exercises, or using exercises that follow a logical sequence that builds up a skill, or that support one another. Sequencing of the presentation of software content may also occur globally between content related to the exercises and content not related to the exercises. For example, in a given day the user may first interact with an exercise, then view a score summary screen, then view a screen allowing the user to select different exercise types, and so on. The timing of the global sequencing may be determined across different days, or across any other period of time (e.g. a sequence of different exercise types and non-exercise content presented during usage in a given day).

The global sequencing of exercise types and non-exercise-content presented to the user may be based on the user's input to the software. For example, upon first use the software may prompt the user to select 3 types of exercises (e.g. hot/cold, breathing, healing color, etc.) that the user thinks they will enjoy the most. The software may then prompt the user to interact with the user-selected exercises more often than the exercise types that the user did not select. Inference algorithms, for example Bayesian inference, may be used to determine which exercise type to present to the user on each day based on which exercises have been most successful for the user, and/or which exercises have been most successful for previous users, and/or which exercises have been most successful for previous users with similar characteristics to the current user. The global sequencing of exercise types and non-exercise-content presented to the user may also be based on a pre-determined hard-coded determination. For example, the software may present an exercise type that trains the user how to use the software before presenting any of the other types of exercise content.

Sequencing may also be provided by the software for content within a particular exercise type. Within-exercise-type sequencing may occur across different levels (periods of for example 5 minutes of interacting with the exercise) of a particular exercise type. For example, upon first use of a particular exercise type, the software may present an exercise level of “easy” difficulty, and then upon subsequent use the software may present more difficult exercise levels. Sequencing of levels may be based by the software on user input, or by a predetermined hard-coded determination. Inference algorithms, for example Bayesian inference, may be used to determine which level to present to the user based on which levels have been most successful for the user, and/or which levels have been most successful for previous users, and/or which levels have been most successful for previous users with similar characteristics to the current user.

Sequencing may also occur for software content within a particular exercise level. Once the user selects a level (or the software selects one for them based on their progress to that point), the user may be provided with a programmed sequence of instructions, or stimuli intended to convey something that the user should do. For example, the user may be provided with the instruction to engage in a sequence of two (or more) alternating mental exercises, each exercise designed to engage the brain's antinociceptive system, and thereby to decrease the user's pain. The stimuli of this sequence, a sequence of two stimuli in this case, may be repeated. After each individual stimulus, or at some point in the sequence, or after the completion of the sequence of stimuli, the user may be instructed to make assessments as described above. The instruction to make the assessments may be provided in advance of the entire sequence, or may be provided during the sequence, or may be made following an individual stimulus. The timing of the sequence of the instructions may be provided by the software, or may be controlled by the user, for example by clicking the UI to receive each additional sequence step.

As the user provides input to the software UI regarding their results with each instruction, these inputs may be used by the software to determine future instructions provided to the user. For example, the user may rate which instructions are the most successful or desirable for them. This information may be stored. This information may be used to preferentially select preferred instructions at a later time, or to avoid less preferred instructions. As another example, instructions that users are more successful at using may be provided in early phases of training, and instructions that users are less successful at using may be provided in later phases of training. Inference algorithms, for example Bayesian inference, may be used to determine which stimulus or instruction to present to the user on each trial based on which stimuli or instructions have been most successful for the user, and/or which stimuli or instructions have been most successful for previous users, and/or which stimuli or instructions have been most successful for previous users with similar characteristics to the current user. This similarity may be based on similarity of answers to characterization questions answered by the user, by the user's pattern of choices in performing the training, or by the user's success in performing the training. For example, stimuli or instructions for the current user may be selected based on their expected success determined by their level of success in prior users who selected other stimuli or instructions similar to the pattern selected by the current user, or who had a similar pattern of success or assessments of stimuli or instructions relative to the current user.

Selection Mechanisms

The software may select the next stimuli, content, or instructions to be presented to the user 10290. This may continue, for example by returning to step 10230. This basic loop may be continuous. The timing and sequencing of individual steps within the loop may vary. In some examples the basic loop may be implemented by the software repeatedly until a stopping point. For example steps in the loop may be repeated by the software in substantially real time.

The software may select or determine different types of stimuli or content or instructions presented to the user through a variety of different mechanisms. The selection may occur at many different levels. Selection may occur on a global level, for example for determining what types of exercises to present to the user, or what types of content to present to the user between exercise sessions. The selection may also occur for software content within a particular exercise, for example the instructions and stimuli intended to convey something that the user should do.

The software may collect information about the user to help select content to present to them. The user may use the software to characterize themselves, for example, they may answer questions through the software or provide information about themselves. This information may be used by the software to make later determinations of content personalization for the user. Inference algorithms, for example Bayesian inference, may be used to determine which content to present to the user based on what content has been most successful for the user, and/or which content has been most successful for previous users, and/or which content has been most successful for previous users with similar characteristics to the current user.

Within an exercise, selection of content presented to the user by the software may be based on inputs from that user or other users. As the user provides input regarding their results with each instruction, these inputs may be used to determine future instructions provided to the user. For example, the user may rate which instructions are the most successful or desirable for them. This information may be used to preferentially select preferred instructions at a later time, or to avoid less preferred instructions. As another example, instructions that users are more successful at using may be provided in early phases of training, and instructions that users are less successful at using may be provided in later phases of training. Inference algorithms, for example Bayesian inference, may be used to determine which stimulus or instruction to present to the user on each trial based on which stimuli or instructions have been most successful for the user, and/or which stimuli or instructions have been most successful for previous users, and/or which stimuli or instructions have been most successful for previous users with similar characteristics to the current user. This similarity may be based on similarity of answers to characterization questions answered by the user, by the user's pattern of choices in performing the training, or by the user's success in performing the training. For example, stimuli or instructions for the current user may be selected based on their expected success determined by their level of success in prior users who selected other stimuli or instructions similar to the pattern selected by the current user, or who had a similar pattern of success or assessments of stimuli or instructions relative to the current user.

Within an exercise, selection of content presented to the user by the software may be determined in real-time based on user-inputs and software algorithms. For example, the user's input may lead to a substantially immediate change in sound level, sound selection, sound quality or parameters, image selection, image opacity, image brightness, image timing or rhythm. Stimuli that are altered in real time by the software may be intended to represent the input being provided by the user, for example representing intensity, quality, or quantity. For example, if a user selects a position along a left-right or up-down continuum on a user interface to indicate the level of pain sensation that they are experiencing, the software may determine a corresponding sound volume or sound pitch to present to the user, and may update the sound presented to the user in substantially real time. The software may also present corresponding image or video intensity or opacity to present to the user, and may update the stimulus presented to the user in substantially real time. The software may also select other stimuli, instructions, words, images, sounds to immediately present to the user. This same process may be used for continua input by the user other than pain or success, including other types of input described for the methods, devices, software and systems provided herein.

User-Based Selection

Selection of content, stimuli, or instructions presented to the user by the software may be directly controlled by the user. The software may allow the user to select a number of settings for the exercises. For example, the user may select the rate, timing or rhythm at which instructions are provided, or at which they perform the mental exercises. The user may be offered a variety of difficulty levels of training, based on their skill level and progress. The user may select a level, for example by clicking an icon on their device screen that may indicate the name or type of training that they will receive or show an image indicating its purpose or nature. The software may provide for the user to directly select the type of exercise content they want (e.g. warm/cool exercise), the number of minutes to practice, and many other settings (e.g. background music).

The software may provide a system to the user for actively controlling the exercise content that the user receives. The software may allow the user to give ratings about the effectiveness of exercise content, or their preferability, and thereby the software may determine the likelihood that the user will be presented that exercise content in the future. The software may allow the user to make this rating in a number of different ways, for example on a 10 point numerical rating scale ranging from “Not helpful” to “Helpful”, or by giving a binary decision (e.g. “Thumbs Up” or “Thumbs down”), or “star” ratings from 0 to five stars. The software may allow the user to make ratings about exercise content on many different levels. For example, the user may rate the effectiveness of a particular exercise type (for example, a preference for the warm/cool strategy), the effectiveness of the settings on a particular exercise session, or the effectiveness of particular trials within a given exercise.

The software may then use these user-ratings to customize the user's future experience. The software and system may make determinations based upon these ratings regarding what output, stimuli or instructions to present to this user or other future users at the present time or at a later time. For example, the software may create a rating measure for each stimulus component based on one or more of the user's ratings of this stimulus component, or other user's ratings of this stimulus component. This rating may be used to determine the timing, frequency, or probability of presenting this output stimulus or instruction to the user, or to future users. Stimuli that have been more highly rated may be presented with higher probability. An algorithm may be provided that seeks to balance collecting input regarding stimuli to assess an accurate determination of reactions to these stimuli and receive resultant ratings, while also attempting to present stimuli which have higher ratings.

Guide-Based Selection

A guide or provider may use the device and/or software to make selections or recommendations on behalf of the user. For example, the guide may recommend exercises for the user to perform. This recommendation may be based upon the user's progress or ratings.

Training, Treatment, Practice, Results

The software may provide the user with repeated content, stimuli, instructions, or training. This may lead in the user to learning, performance improvement, neuroplasticity, changes in symptoms 10310.

### FIG. 16. Example Flowchart

The system and software may provide a system for improving a mental state, shown including steps shown in FIG. 16. Each of these steps may optionally be taken or excluded, and the order may be varied. The system overall may constitute a loop, so the order of the steps progresses repeatedly rather than in a linear fashion. Also, some steps may happen out of sequence based upon the choices made (eg the user or guide may make changes to the UI that affect the flow). The device/software 1300 may interact with the user 1100 as well as one or more guides or providers 1500. This interaction may take place by communication network or in-person, and may use a variety of communication technologies available, including text messaging, audio or videochat, screen sharing, or use of UI elements to make selections or receive information. The software device 1300 may provide the steps shown in FIG. 15, shown for device/software 10200.

In this diagram, vertical arrows may represent flow through the steps, while horizontal arrows may represent flows of information between elements or processes. The control software and device 1300 may perform many steps either independently, or may be guided by or controlled by either the user 1100 or the guide and/or provider 1500.

An early step may be the selection of the target condition to improve in/for the user 1310. For example, a user in pain may select 1110 that they want to improve/decrease their pain. The guide/provider may also select 1510 that they want to improve the user's level of relaxation. The software may select 1310 that the user increase their focus. The user and/or provider and/or guide may select one or more of these conditions using a UI provided by the software/device.

The software may characterize the user 1320 in a variety of ways. In one embodiment, the software may characterize the neurotype of the user, indicating elements of the user's predicted brain function or structure. The software may provide questions for the user to answer, or may collect physiological data about the user, including brain or neurophysiological data including but not limited to EEG, fRMI, MEG, EMG, GSR, heart rate and pattern, breathing rate and pattern. The software may categorize the user based on similarity to a database of past users, for example selecting their Neurotype.

The software may select and customize content 1330 to present to the user. This content may come from a store 115. This content or stimuli selected may take a variety of forms, including but not limited to:
1) Instructions; a) The instructions may be provided in a variety of formats including; i) Audio; (1) Pre-recorded audio; (2) Live audio provided in person by the guide/provider; (3) Live audio provided by communication means eg; (a) Telephone; (b) Electronic: Chat, audio chat, video chat, messaging, email; ii) Video (pre-recorded or live); (1) Person speaking the instructions; (2) Person demonstrating the instructions; (3) Animated or filmed representation of the instructions; (4) VR representation of a situation analogous to the instructions; (5) Video chat (for example webRTC-based video conference); iii) Group instructions (for example on a conference call or video conference call); iv) Text; v) Images; vi) Other forms of instruction, including; (1) Games that the user engages in; (2) Puzzles or other tasks; (3) Cognitive training tasks that the user engages in; b) Instructions may indicate to the user a variety of tasks that they are to accomplish. These tasks may include; i) physical tasks; (1) exercise, body or yoga postures or sequences, or sleep regimens; (2) following treatments plans such as taking medications, following up with providers, maintaining abstinence; (3) diet recommendations; (4) checking in to verify compliance with tasks; ii) mental tasks; (1) focusing attention on; (a) a part of the body; (b) tactile sensations, or imagined tactile sensations; (c) emotions, or imagined emotions; (d) visual images or scenes; (e) real or imagined movements or tasks or body postures; (2) imagining taking an action, such as imagining something in the realm of; (a) visualizations; (i) imagine a color filling an area of your body; (ii) imagine a scene, including a pleasant or favorite scene; (iii) imagine; (b) tactile; (i) feel actual tactile sensations from an area of the body; (ii) create imagined tactile sensations in an area of the body; 1. for example imagine water flowing over or through that area; 2. imagine fire in an area of the body; (c) affective/emotional; (i) imagined positive emotions such as freedom, happiness, relaxation, satiety; (ii) imagined negative emotions such as sadness or depression, anxiety, craving, pain, fear, hatred, aversion; (d) auditory; (i) real or imagined (created) sounds such as a pleasant tone; (e) gustatory; (i) real or imagined tastes such as a favorite food or taste that evokes positive memories; (f) olfactory; (i) real or imagined smells such as a favorite small or smell that evokes positive memories; 2) Stimuli; a) Video, images, VR, videogames, sounds, speech, music; b) Audio; i) Music; ii) ‘Binaural beats’; iii) Relaxation stimuli; 3) Feedback; a) Representations intended to correspond with the user's experience of their own progress or internal state (for example getting more intense as the user rates their internal experience to be more intense) including; i) Videos, including partially transparent videos with transparency adjusted as feedback; ii) Sounds, including noise, sounds of water, fire, speech, breathing, Shepard tones; iii) Images; iv) Video game or animated elements; v) Selection of virtual scene elements; 4) Information; a) Scores or representations of the user's success including: i) Measures of targets that the user has achieved, such as the level to which they have improved their mental state, the duration of improvement, the maximum improvement, or a combination or multiplication or addition of these; b) Predictions of their future success (such as the improvement in their mental state, for example pain) including; i) Based on their behavior and/or success thus far the predicted improvement that they are expected to have based on results with other people or similar other people, or using similar stimuli or instructions or strategies; c) Predictions of which content or stimuli or instructions are recommended for them; i) Based on their past success with these content or stimuli or instructions; ii) Based on their ratings of these content or stimuli or instructions; iii) Based on their similarity to other people, and average or predicted responses to content or stimuli or instructions based on results in previous people;

The selection may also be made by or influenced by selections made by the user 1130 or the guide/provider 1530, for example who may choose which sets of instructions to use from UI elements in the software.

The selection may include selection of one or more stimuli, content elements, instructions, brain postures or training exercises, mental exercises, mental rehearsal instructions (optionally in one or more sequences) thought to be desirable for the subject, for example based upon the user's neurotype or characteristics. This may include:
Customization by the subject, or by someone else for the subject including the guide or provider, or automatic customization by software including, but not limited to: 1. User self-optimizes the training; a. Software changes length of pause, preferred music, preferred nature sound, preferred video that are played during training based on user choices; 2. Selecting audio based on history of user's responses; a. Software may select different audio tracks, e.g. different spoken instructions, based on which tracks have been most successful in decreasing users pain or other experience ratings in previous trials; b. Software may select different posture sequences, e.g. different sequences of spoken instructions, based on which sequences have been most successful in decreasing users pain or other experience ratings in previous trials; 3. Software may customize the content provided to subject, including sequences and instruction tracks, based on inputs of the subject; a. Location of painful area; b. Mood; c. Extensive surveys (e.g. neurotype); 4. Automatically modulating focus period length based on subject's performance; a. e.g. making it shorter if users indicate “I spaced out”; 5. Saving and restoring user's preferences across sessions, specific to individual exercises (preferred video, background music, volume settings);

Content may be presented to the user 1340, for example via a presentation device or display 140, 160, 170, which may include a computer, mobile device, or other device. This content may be presented, for example, on a screen such as the Training Screen shown in FIG. 2. The user may receive and perceive this content, for example listening to instructions or viewing or listening to stimuli. The user may then be instructed to perform a task. For example, the user may be instructed to direct their attention toward particular aspects of a presented stimulus; the user may be instructed to direct their attention toward particular aspects of their own subjective experience (for example their tactile sensations from one body part); the user may be instructed to form a mental image or construct (such as imagining the feeling of water flowing over a body part); the user may be instructed to control a bodily function (such as to breath at a certain rate).

The user may be instructed to perform a task or follow an instruction. The software may time the user 1350. The timing may be controlled by:

A variable length pause during which the user may perform a cognitive or other task, behavior or receive a stimulus. The variable length of this pause may be selected by a method that may include (but is not limited to):

a. Selection by the subject or provider/guide, for example by selection of a number of seconds using a slider; b. Selection for the subject, for example based upon the subject's neurotype, behavior, or the based upon duration required by or used by the subject on previous trials, or based upon the duration required by or used by other previous subjects on previous trials, including other previous subjects deemed to be in some way similar to the subject, for example based upon their neurotype, behavioral performance, or activity measurements; c. Estimation of an optimal or desired length for the subject, for example based upon a length that lead to desired outcomes on previous trials in this subject, or in other previous subjects.

The user may then perform the instructed task 1150, or be guided in doing so by the guide/provider 1550, and the user may be timed in doing so.

The software may then provide an indication that the user should become aware of their own experience or internal felt sense 1360, and/or a duration to focus their awareness on this. This indication may be generated by the control software or by the guide/provider 1560. For example, the user may focus their awareness on the effects of the content, stimuli, or instructions provided to them in 1340. In one example, if the user is provided instructions for relaxation, the user may estimate their level of relaxation or stress/anxiety, before, during, and/or after performing these instructions. The user may also be aware of and make continuous estimates of their mental state during any part of this process or all of the process. In another example, the user may continuously monitor their level of pain throughout this process, indicating their perceived level of pain using a UI element, for example the vertical line 450.

The software may receive the user's rating 1370. The software may also provide this to the guide/provider, either in real time or later, or in summary form. The user's ratings or responses may be captured by a variety of methods including mouse 220, voice 230, physiological measurement device 240, touchscreen 160 or other methods. The user may provide responses indicating their experience, responses indicating their ratings, or responses indicating their internal state, or physiological processes of the user may be measured (250, 260, 270). These may include: Measurement of the subjects response or performance or activity. This may include (but is not limited to):
a. A pain rating or subjective rating; b. Determination of the duration that the subject took in performing a trial; c An activity measurement; d. A measurement of physiological activity; e. Measurement of a subject's response by keyboard, touchscreen, button or virtual button press, one or more slider inputs; f. Measurement of a subjects' voice or voice commands, including but not limited to voice recognition; g. Measurement of a subjects' breathing rate or pattern. This may include determining the breath using audio, such as amplifying the sound of the subjects' breathing using a microphone, including the microphone of a mobile phone or device. This may include determining the breath using imaging, such as using a light and camera or input, including the camera of a mobile phone or device (for example placing a finger over a light and camera simultaneously to measure color or intensity changes associated with breathing rate). This may include determining the breath using an accelerometer, such as using an accelerometer to measure movements associated with breathing, including the accelerometer of a mobile phone or device; h. Measurement of a subjects' heart rate or pattern, skin conductance, EEG or other physiological response. This may include determining the heart rate or pattern using audio, such as amplifying the sound of the subjects' heart rate or pattern using a microphone, including the microphone of a mobile phone or device. This may include determining the heart rate or pattern using imaging, such as using a light and camera or input, including the camera of a mobile phone or device (for example placing a finger over a light and camera simultaneously to measure color or intensity changes associated with heart rate). This may include determining the heart rate or pattern using an accelerometer, such as using an accelerometer to measure movements associated with heart rate or pattern, including the accelerometer of a mobile phone or device.

The software may store the user's inputs, actions, score, success ratings an other information 1380, for example into memory or into a database 130. This information may be stored in a database on the users device and/or on a server or elsewhere. This information may be used to compare the user's results with prior sessions or future sessions of the same user, or with other users, or with aggregated data from other users (which the data may be added to).

The software may then provide feedback representing the user's responses 1390. A variety of types of feedback were described in FIGS. 1 and 2, for examples see feedback 500. This user feedback may also be controlled or influenced by the guide/provider 1590. The feedback may be perceived by the user 1190. This process may allow the user to clearly perceive their own state or progress through this process, which may be useful for example in learning greater awareness, being precise, and motivating the user's progress. The software may store all data related to this process for the future 1400. The provider or guide may review that data 1600 either during the course of training of the user (live) or at a later time. This may be useful in the guide or provider selecting what course of action for the user to take, selecting next content 1610, or being aware of or diagnosing the state or progress of the user.

The software may make determinations 1405 based upon the users input that may be used to guide additional steps. These determinations may also be made or guided by the guide/provider 1605. For example, the software may compare the users result with their expected result based upon past behavior of the user, or past behavior or other users. These determinations, which may include statistical inference (for example Bayesian inferences of the best course of action to take or best stimuli or instructions to use) may be used in further selections for the user, or for future users.

Determinations may include adaptive tracking of the stimuli based upon the users performance. This may allow the software/device 1300 to function as a game, or neurogame. One method of setting and continuously adjusting performance targets is to use adaptive tracking. In this methodology, an initial performance target may be set to a value that it is anticipated that the user may be able to achieve. Using adaptive tracking the performance target may be made more challenging when the user achieves some number of successful trials in a row, such as three. The performance target may be made less challenging when the user fails to achieve success on some number of trials in a row, such as one. Other methods of adaptive tracking are familiar to one skilled in the art. When the performance target is made more challenging, the user can be alerted that they have moved up to a more challenging level, and when it is made easier they can be alerted that they have been moved down to a less challenging level. The user's goal, of course, is to achieve the higher levels.

The software may then select or customize the next content, stimuli or instructions 1410. For example, the software may engage in:
Determination of a next one or more content elements, stimuli, instructions, brain postures or training exercises (optionally in one or more sequences) thought to be desirable for the subject optionally based in part upon the measurement of the subjects response or performance or activity. This may involve (but is not limited to):
a. Selecting one or more content elements, stimuli, instructions, brain postures or training exercises that lead to a decrease or change in the subject's pain rating or subjective rating.
b. Selecting one or more content elements, stimuli, instructions, brain postures or training exercises that lead to a change in the subject's activity measurement or physiological activity, or duration that the subject took in performing a trial.
c. Determination by computer algorithm, by artificial intelligence, statistics or Bayesian statistics

The software my continue this process by repeating the loop, for example starting again at 1340. This continuation may continue until a variety of stopping points, including the user indicating their readiness to stop, the guide or provider indicating stopping, or a time expiring as measured by the software.

At this point the software may carry out an end sequence and or final questions 1420. This may include one final set of steps similar to 1340-1410. It may include asking final questions to the user or receiving ratings or input. It may include providing scores or other feedback or information to the user. It may include positive rewards including presenting desirable stimuli, monetary rewards, or points.

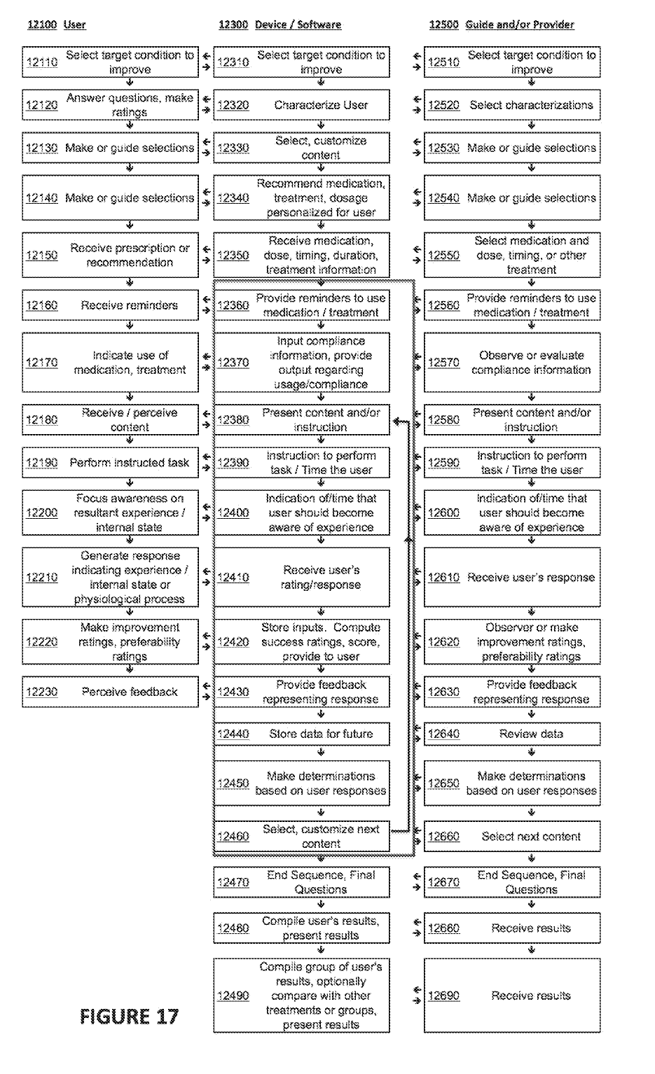

### FIG. 17. Example Combination Treatment Flowchart

The methods, devices, software and systems 12300 provided herein may be performed in combination with pharmacologic intervention, medications, medical devices and procedures. The methods, devices, software and systems provided herein may also be used in combination with pharmacologic testing or medical device or procedure testing. An example is provided in FIG. 17. FIG. 17 shows an example process similar to FIG. 16, and adds steps related to medication or treatment, including steps 12340-12370, 12140-12170 and 12540-12570, 12480, 12490, 12680 and 12690.

Use in Combination with Particular Medications and Procedures

The software may include a stored list of medications, with corresponding settings and/or stimuli, based on the individual medication, the therapeutic area, the indication, or other factors designed to match the medication or treatment with the software's stimuli or instructions. Stimuli, exercises, or instructions may be selected to match with a particular medication, for example a medication entered by a user 12100 or guide 12500, or with a class of medication.

An example list, indicating example associations between medications and medication classes and treatment categories targets, which may be used to determine selections of stimuli, exercises, or instructions based on treatment category for each medication, is provided in Table 1. An example list, indicating example associations with disease or psychological conditions and categories or treatment targets, which may be used to determine selections of stimuli, exercises, or instructions for each category, is provided in Table 2. This information may be stored and accessed by the software. In this way, the software may select an appropriate set of stimuli, content, exercise or instructions for a user receiving any of the listed medications. Further, the software may select an appropriate set of stimuli, content, exercise or instructions for a user suffering from any of the listed conditions.

The software may provide stimuli, exercises, training or instruction for individuals receiving any of these medications or treatments. The type of stimuli, exercises, training or instruction may be selected based upon the class of the medication or treatment 12340 by the software 12300. The stimuli, exercises, training or instruction may be selected by the software based upon prior data for other individuals with similar conditions or receiving similar indications or treatment, or indicating the results or efficacy for those individuals. Recommendations my also be made by a guide or provider 12540, or by the user 12140 and entered into the software UI. The software may receive medication dose, timing, duration, or treatment information 12350 (and this may take place prior to 12340). The provider may select the medication, dose, timing, or other treatment 12550. This may by upon recommendation made by the device and software 12300, for example based upon information about the user collected in 12310, 12320, 12330. Reminders to comply with treatment or take medications may be provided by the software 12360, and/or may be provided by messages entered into the software by the guide 12560 and presented to the user 12160. The user may indicate use of medication or treatment compliance 12170 and input this information into the software 12370. This information may also be communicated by the software to the guide or provider 12570, or integrated into an EMR system.

Treatment Efficacy Testing and Prediction

The software may also be used in combination with treatment efficacy testing. For example, the software may be used to monitor the progress, symptoms, compliance or other information about users in a clinical trial, and to then compile resultant data on their outcomes. As described in FIG. 17, the software may closely monitor users, and this information may be useful in gathering clinical trial data. This may be useful in clinical trials of treatments of a variety of types, including cognitive interventions, medications or pharmaceuticals, diets, medical device treatments, medical procedure treatments such as surgeries, etc.

The software may be used to compute average responses over time for each user before and/or after a treatment, so that the responses to different types of treatment may be compared. Through the software sorting this data based upon characteristics of the user, it may also be possible to determine the efficacy of different types of treatments for different types of users. The software may allow for a prediction of which treatment will be most effective for a particular user, based on the characterization of that user, and the responses of users with a similar characterization in prior data.

The software may use a variety of types of data for the characterization of users, and for grouping of users to compute responses to any form of treatment, alone or in combination with the provision of stimuli, instructions or training provided by this software. Examples of the types of data that may be used to characterize individuals include genetic data, disease risk data, family history of a condition, brain imaging data, neurophysiological data, questionnaire data, performance data, medication data. The software may compile the results for a user 12480, and may present these results to a guide or provider 12680. In addition, the software may compile results across users, and optionally compare results across groups 12490. This information may also be provide by the software to a provider or guide 12690. This may allow the provider or guide to monitor the progress of a user or patient, or group of users or patients, and to compare them with other groups. For example, the software may allow for the comparison of improvement of groups of users who receive different stimuli, exercises, or instructions, or who receive different treatment of other forms, for example medications or other treatments, and who are monitored using the software. The software may also allow for prediction of the success of a user based upon their characterization, and the results from prior users with similar character. For example, the software may provide a quantitative prediction of the improvement that a user is expected to achieve, based upon results from previous users with a similar condition, receiving similar treatment, receiving similar stimuli, content or instructions. In addition, the software may indicate to a user which elements of their characterization, if changed, would lead to improved predictions, by re-computing predictions with different values of the user's characterization. For example, if a user has filled out a characterization questionnaire and indicated that she only receives 6 hours of sleep per night, the software may provide a prediction of the user's improvement in pain or another condition if this stays the same, and the software may compute the prediction of the user's improvement in pain or another condition if the user makes a change, for example increasing this to 8 hours of sleep per night. The software may in this way determine which aspects of the user's characterization, if changed, would produce the greatest improvement in the user's predicted outcome.

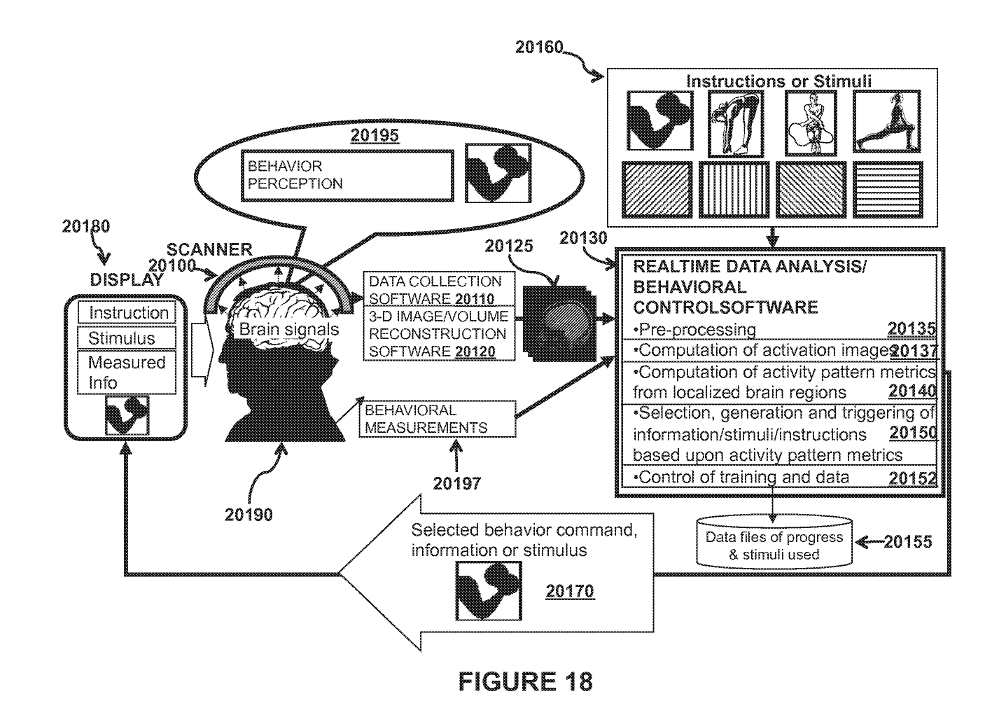

### FIG. 18. Example Involving Physiological Measurement

As illustrated, a scanner and associated control software 20100 initiates scanning pulse sequences, makes resulting measurements, and communicates electronic signals associated with data collection software 20110 that produces raw scan data from the electronic signals. The raw scan data is then converted to image data corresponding to images and volumes of the brain by the 3-D image/volume reconstruction software 20120. The resultant images or volume 20125 is passed to the data analysis/behavioral control software 20130. The data analysis/behavioral control software performs computations on the image data to produce activity metrics that are measures of physiological activity in brain regions of interest. These computations include pre-processing 20135, computation of activation image/volumes 20137, computation of activity metrics from brain regions of interest 20140, and selection, generation, and triggering of information such as measurement information, stimuli or instructions based upon activity metrics 20150, as well as the control of training and data 20152, using the activity metrics and instructions or stimuli 20160 as inputs. The results and other information and ongoing collected data may be stored to data files of progress and a record of the stimuli used 20155. The selected instruction, measured information, or stimulus 20170, is then presented via a display means 20180 to a subject 20190. This encourages the subject to engage in imagined or performed behaviors or exercises 20195 or to perceive stimuli. If the subject undertakes overt behaviors, such as responding to questions, the responses and other behavioral measurements 20197 are fed to the data analysis/behavioral control software 20130.

### EXAMPLES

### User-Created Content Offerings

Users may offer content that they have created to other users. For example, a user may create new stimuli, sequences, or training programs. These may be used by themselves within the software. This user-created content may also be presented to other users. User-created content may be presented to other users for free. User-created content may be presented to other users for a fee. In the instance where user-created content is presented to other users for a fee, the software may track the payments and billing. The software may also maintain ratings of creators of other content, and collect user comments and ratings for presentation to other users, so that users may decide which content they most want. The software may make it possible for a user to sort through user-created content based on search algorithms including keyword searching, searching by user ratings, or searching by tags. The software may also make possible revenue-sharing or profit-sharing, for example providing a fraction of revenue or profit collected from users who use content with the content-creator, and/or a fraction with the content-distributor, and/or fraction with a marketing affiliate, and/or with a provider including a healthcare provider or an insurer. Each of these contributors may add value by modifying content, personalizing it, endorsing it, or selecting content to be presented to an individual user, for example based upon the user's characteristics or progress.

### Mobile Devices

The software may be provided on a mobile device, including a smartphone, tablet, smartwatch, or other mobile connected device. In addition, the device may be used to collect measurements from the user, based on whatever measurements the device is capable of making. These measurements may be stored along with other aspects of the user's actions, and tracked over time. Conversely, the progress of the user using this system may be provided to other systems or uploaded to medical or other record keeping systems, for example EMR's, or personal health information tracking systems. Some of the variables that may be recorded by a mobile device include: heart beat timing and rate, breath timing and rate, GSR, accelerometer data, geolocation data, temperature. Other data available by connected devices may also be used in coordination with this information. For example, a user's progress may be inferred or scored based on changes in any of these parameters.

### Biofeedback/Neurofeedback

This information may also be used in the context of biofeedback. For example, heart rate or breathing rate or EMG or EEG information captured by a mobile device may be input into the software. This biological or other information may be used in addition to or in lieu of the user input described.

### Sleep Induction/Insomnia

The software may provide stimuli, content, instructions that may be provided to a user for the purpose of inducing or maintaining sleep. For example, the software may provide light or sound that modulates at a rate similar to the user's breathing rate. The light may be provided by a device light or LED, but the brightness of content on the screen, or otherwise. The user may be instructed by the software to breath matching this rate. This rate may be decreased by the software over time, decreasing the user's breathing rate. This may encourage sleep. The user may select the breathing rate for the software to use. The software may also select an appropriate breathing rate for the user based upon the user's characterization. The breathing rate used may be stored by the software for use later. The software may match the user's measured breathing rate. The software may also provide audio instructions, for example verbal calming instructions, to help a user to sleep.

### Non-Conscious Stimuli or Instructions

The software may provide stimuli, content, instructions that are delivered to the subject in a non-conscious fashion. For example, the stimuli may be delivered by the software below, or just below, the user's perceptual threshold. This may be accomplished by the software using visual or auditory or other stimuli. The software may accomplish this using masking stimuli that are more salient, either at the same time or at a closely adjacent time, to mask a stimulus that is being presented non-consciously. The software may also provide stimuli or instructions containing patterns or sequences or material for a user to learn that the user is unaware of. In this way, it may be possible for the software to train the user without the user's awareness. The software may provide non-conscious classical or other conditioning methods.

### Mobile Devices Used for REM Sleep Capture, Stimuli, Lucid Dreaming

The software may determine algorithmically when a user is in a state of REM sleep. This may be based upon measurements of heart rate, eye movements, body movements, EMG, breathing rate, accelerometer movements, EEG, or other parameters. For example, the Basis watch is an example of a mobile wearable device that provides realtime estimation of sleep cycle (light, deep, REM) based on such data and algorithms.

The software may provide stimuli to the user based on the user's sleep state. For example, the software may provide stimuli to the user until they reach a state of sleep. The software may provide stimuli to the user during an interruption of sleep. The software may provide stimuli to the user based on up the user's sleep state or state of wakefulness or arousal. An algorithm and store of different stimuli may be used to provide different stimuli or different types of stimuli based upon the user's state of arousal or sleep.

The software may provide any of the types of stimuli described herein based on the user's state of sleep. In particular, the software may provide relaxing stimuli with the goal of helping a user to go to sleep. These stimuli may include sounds or light or images that cycle slowly and encourage the user to match their breathing rate to these stimuli. The rate of change of the stimuli may also decrease to bring a user to a state of low arousal and slow breathing where they may more easily fall asleep. The software may also use other types of visual stimuli, auditory stimuli, or tactile stimuli including taps or vibrations.

The software may be used to train a user to induce lucid dreaming. The software may determine the user's sleep state, and use this information to determine when to present stimuli to the user, or when to stop presenting stimuli to a user. For example, a user may be trained during wakefulness to recognize a certain stimulus as an indication that the user is sleeping. This stimulus may include a sound, a tactile stimulus, a visual stimulus, other stimuli, or a combination. The software may then present this stimulus or combination while the user is determined to be in an appropriate sleep state, for example in REM sleep or a sleep transition. This may allow for the user to recognize this stimulus and determine that they may be sleeping, and to achieve lucid dreaming. The software may continue to present the stimulus until it is recognized that the user has become lucid. This recognition may be due to changes in physiological signals, or changes in user motion. These stimuli may include any of the types of stimuli described herein. In particular, the stimuli may include vibration provided by a wearable device such as a watch, sound provided through earphones or a speaker, visual stimuli provided by a screen or light, or visual stimuli provided by lights or LED's in a sleep mask worn by the user.

Example steps: user wears smartwatch; smartwatch or other monitoring device detects REM sleep; smartwatch or other monitoring device monitors other inputs such as heart beat or breathing; smartwatch indicates this to paired smartphone or other device; smartphone presents auditory stimulus to user, potentially stimuli linked to breathing or heart beat or other biological signals; smartphone presents visual stimulus to user through visual mask or other output means. Through these steps, a user may be trained to recognize REM sleep.

### Microinstructions/Delivery

The software may provide short stimuli or instructions to the user that may serve as a short session or reminder. For example, the software may provide a single short instruction for the user to perform a single short task, such as visualize a relaxing scene. These short instructions may be provided to the user while they are not otherwise interacting with the software. For example, the software running on a server may provide a short stimulus or instruction to the user by email, SMS, digital message, audio message, voice message or otherwise. The timing when the stimuli are sent may be selected or stored by the software. The software may send reminders to the user to re-engage with a session or training.

Communication with Other Users

The software may provide users with community or social network functionality to allow users to be motivated or reminded by other users to perform desired tasks, or follow intended instructions.

An example of a community of this sort for messaging with a listener is 7 Cups of Tea. User's may be provided with the opportunity to interact with trained guides or volunteer users by text messaging, voice, video, or otherwise. The user's may ask questions, be provided with answers, be provided with guidance.

### Guides, Simulcast

Guides may provide instructions to users, or to groups of users by providing stimuli, instructions, or commentary. This may be provided by email, text message, SMS, audio message, video message, or using one-to-many or many-to-many simulcast or conference calling functionality. The software may provide a guide with real time feedback regarding the progress of one or more user, such as their position in training, their score, their responses or other elements of what the user may be experiencing, or a screen share. The guide may be remotely located, and may instruct or help or motivate one or many users.

### Pairing Users for Training, Teams

The software may allow users to interact with other users in a variety of different ways. The software may allow groups of users to form online “teams”. The software may select individual users to invite to a particular team, or allow users to select and invite other users to their team through an online forum created for such purpose. The software may select groups of users to be on the same team based on the shared similarity of characteristics of those users, or on any other probabilistic algorithm for determining likelihood of team success and individual team member success. The size of the team may be determined either by the software or by individual team members.

The software may include many features designed to enhance success of team members. The software may provide team goals or challenges that each member of the team would work towards. For example, the software may set a goal for a team to achieve a set number of team total points in 7 days; the team total points may be the sum of the points of each individual team member. The software may also provide tools for team members to communicate with each other. For example, the software may allow team members to send private or group messages to other team members encouraging them to achieve their group challenge. The software may award prizes for completion of team goals. For example, the software may award badges, virtual gifts, monetary gift cards to each team member of a team that successfully completed its goal. The software may allow teams to compete for prizes. For example, the software may award a gift card to the team that scores the most points in a week time period.

The software may allow users to interact with each other in real-time during exercises. The software may allow users to compete in real-time while practicing the same exercise. For example, two (or more) users may attempt to simultaneously match the timing of a set cycle of switching between two mental states; on each cycle, the user that matched the timing most closely would be awarded the most points.

The software may allow users to cooperate in real-time while practicing the same exercise. For example, two (or more) users may attempt to simultaneously match the timing of a set cycle of switching between two mental states; on each cycle, all users would be awarded would be awarded a points-multiplier based on the difference between the correct timing and the average timing of all users. In another example, one user may set a pace of switching between mental states; in real-time another (or more) user(s) may try to match the set pace, and points would be awarded based on how closely the timing of the two (or more) users matched.

Paired Breathing or Paired Mental Exercises

The software may provide for two users to receive stimuli indicated each-others breathing, for example recorded audio, or visual information indicating the rhythm or pacing of breathing, or animations indicating where in the breath cycle each individual is. The software may allow two individuals at different locations to learn to synchronize their breathing, or to perform breathing exercises together, but presenting this information, which may be transmitted via network. In an example, user1 produces a rhythm of breathing, the software may receive this rhythm as input from user1, for example by recording user1's breath audio accelerometer or abdominal displacement measurements or receiving UI clicks from user1. The software may represent this information visually to user1 on a screen, for example as an animated object that expands and contracts in size in time with user1's breathing. The software may also represent this information in sound to user1, for example with sound becoming louder along with the pattern of breathing, or with the triggering of different sounds based on the different phased of breathing (in, hold in, out, hold out). This visual or auditory information or other information may also be presented by the software to user2, who may be in a remote location. User1 may also receive similar information from the software regarding user2. In this way, the two user's may be able to synchronize their breathing.

Similarly, two or more users may be provided by the software with information regarding each of their mental states or progress through a mental exercise or sequence. For example, when user1 completes a step of a mental exercise such as imagining a warm sensation in inputs this into a UI, this information may be represented to both user1 and user2. When user1 rates their experience or perception, such as their pain, this information may be provided to both user1 and user2, but information on a screen, or audio (including sound intensity), or otherwise. User1's score may also be provided to user2. In this way, some or all of the information from one or may be shared with one or more other users. This may allow for cooperative exercises, or competitive exercises. For example, the software may allow for user1 to perform one step in a mental exercise, and then provide this information to user2, so that user2 may perform the next step in a mental exercise. Alternatively, the software may provide for two users to perform two or more steps in a sequence concurrently, such as alternating back and forth between two steps, while being able to know which step the other user is on. The software may provide for users to see each other's timing, or to ‘race’ to see who completes steps more quickly, with more even time packing, or receiving a better score. The software may also provide for this across a plurality of users.

### Mental Rehearsal/Practice as Therapy

The software may provide for instruction in mental rehearsal or practice. This may be provided by the software as a form of therapy. For example, the software may provide instructions for a user to practice, or to mentally rehearse, a mental exercise repeatedly. The software may instruct the user to mentally rehearse an exercise to activate a given mental, cognitive or brain system, such as the antinociceptive system. This may provide learning, plasticity, or improvement in the user's abilities. This may have positive therapeutic effects for the user.

### Moving Stimuli/Eye Movements

The software may provide stimuli to induce in a user exercises related to eye movements or movements of attention. For example, the software may provide visual stimuli for the user to track using eye movements, such as a target that smoothly moves back and forth on a display screen, or that moves from location to location on a display screen, or using a succession of stimuli designed to keep the user's eyes moving and following. Stimuli may also be provide for the user to focus upon different areas of other sense domains, for example using tactile stimuli on alternating sides or alternating points on the body, or sounds of alternating or changing frequency or stereo location. The software may instruct the user to allow memories to arise during engagement with these stimuli, or to focus on bodily sensations or emotions.

### Cognitive Therapy

The methods, devices, software and systems provided herein may be used in combination with cognitive therapy, cognitive behavioral therapy, dialectical behavioral therapy, or other forms of psychotherapy or psychological therapy. For example, users undergoing any form of therapy may be provided with software for training during a session, or for training between sessions. The training instructions or stimuli provided by the software may include elements taken from any of the forms of therapy mentioned or others. In this way, the software may provide a computer-controlled version of leading the user through exercises similar to those used in traditional forms of therapy. In addition, the user may be presented with stimuli of watching other users or the user participating in therapy, for example watching sessions recorded through audio or video. The user may be instructed to imagine themselves in the situation presented, or participating in the exercises being presented.

### Pseudo Measures of Internal Actions

The software may allow the user to indicate their internal actions, internal felt experiences of sense using a pseudo measure intended to indicate their internal state or activities. For example, the software may allow a user to indicate when they perform an internal task or have an internal experience by selecting a UI element that indicates what experience they are having, or when it starts or stops. The software may allow users to indicate the pacing or rhythm of their experience by the pacing of UI element selection. The software may allow a user to indicate other aspects of their internal experience, such as its vividness, or intensity, or their ability to achieve an internal goal, task, perception or experience. The software may allow users to indicate this through selecting a button or UI element (e.g. low, medium, high buttons), a slider, a screen position, or other input elements. The software may allow the user to match their internal experience to a range or a selection of sensory stimuli that they may choose between, or adjust the parameters of. For example, if a user's pain or other sensation feels hot the user may be allowed to choose images or video or animations or stimuli representing heat, or the degree of heat they are experiencing. If a user's pain or other sensation or experience feels intense to the user, they may be allowed to indicate the level of intensity by matching it to a scale, or the loudness of a sound, or by selecting attributes of what they feel.

### Artificial Intelligence and Avatars

The software may allow the user to interact with a virtual avatar such as a virtual instructor, teammate, coach, guide, or collaborator. This may be provided as part of a multi-player scenario. The virtual avatar may simulate the interaction with a real person, to make the experience more engaging. The virtual avatar may be presented through text, chat, audio including spoken audio, text to speech, animation, video, or using other means to simulate an interaction with a person, animal or other entity. The virtual avatar may provide encouragement, motivation, instructions, score, or other elements or stimuli. The software may provide a chatbot that allows a user to have a simulated communication with a virtual avatar. The user may use an avatar to represent themselves within the software, or to represent other individuals or entities. The content or stimuli presented or created by a chatbot or AI or avatar may also be mixed with content or stimuli presented or created by a human, or personally created for an individual user, for example in response to their questions, comments, or progress.

Continuous Experience Tracking of User

The experience of a user may be continuously monitored by tracking the user's continuous UI input, for example using continuous tracking of the user's screen selection point for a period of time. The software and UI may use the screen position as the basis for understanding the representation of the user's internal experience. The software and UI may also use the velocity, change in velocity, or change in direction of the users selection point on a screen to indicate the user's choices. For example, the user may indicate that they have completed a step by changing direction, or by crossing into a defined region of the screen. A user may indicate their level of success or intensity of experience by the position of their selection on the screen or by the amount or direction that they move their selection point, or by the velocity with which they move it. These gestures may also be accomplished without a screen or using other UI controls such as a game controller, touch screen, accelerometer movements, or hand or body part motion tracking.

Selectable Delay Period

The software may provide a delay after the completion of a stimulus that allows a user to receive or perceive the stimulus or to perform a task. The delay period duration may be adjusted by the software. This adjustment may allow the user to select a desired delay period. The software may select or store a delay period for each step, sequence, instruction, stimulus or exercise. This may be personalized for a user, for example by multiplying the standard delay period value by a constant selected by the user. The delay period for the user may also be selected by measuring the time until the user indicates that they are done with a stimulus, task, or instruction, or that they are ready to proceed to the next one. These values may be stored for the user in order to optimize the duration of the delay period in future presentations. In some examples, the duration of the delay period may be 1 seconds. In some examples, the duration of the delay period may be 10 seconds. In some examples, the duration of the delay period may be 1 minute. In some examples, the duration of the delay period may be 10 minutes. In some examples, the duration of the delay period may be about 600, 120, 30, 15, 10, 5, 4, 2, 1, 0.5, 0.2, 0.1, 0.01, 0.001, 0.000001 seconds.

Continuously Recording User's Input, Converting into Stimulus Intensity: Opacity, Volume, Speed

The user's input may be input continuously for a period of time by the software, with the UI or stimulus parameters controlled in substantially real time by this input. For example, if a user indicates the intensity of their experience by the selection position of a controller or on a screen, this may be determined by the software and converted in real time into the parameters of a stimulus. For example, the user's selection may be determined and converted in real time into the volume of one or more stimuli that are being presented, or the opacity of one or more image or video or visual stimuli that are being presented, or the speed that a stimulus moves or is animated.

Two (or More) Countercurrent Video, Audio or Stimulus Streams

The software may provide two (or more) stimuli intended to convey different features of a user's experience. These stimuli may be presented in a counter-current fashion to the user, based on the user's input. For example, if the user is indicating their level of hot vs. cold, a video with sound representing hot and a second video with sound representing cold may be presented by the software. As the user indicates their internal experience by selecting a screen position, the software may determine this position and thereby determine the user's experience and represent this to the user by increasing the opacity and audio volume of one video while simultaneously and correspondingly decreasing the opacity and audio volume of the other stimulus.

### Help

The software may provide help to a user when they request it. For example, the software may provide special instructions or exercises when users performance does not meet a threshold level, or when the user requests help. The help functionality may be provided as a separate exercise or sequence or level that may be selected by the user or selected by the software to provide instructions or exercises to help a user. In addition, the difficulty of steps, levels, exercises may be adjusted to fit the user's abilities or performance. The software may provide help or suggestions if the user has not interacted with it for a period of time, which may indicate inattention.

Measuring Initial, Final and/or Target Pain/Symptom Level

The software may provide a means for the user to input an initial rating of their experience prior to a session or training, for example pain, sadness, focus, anxiety, craving or other measures. The software may provide an input for the user to indicate the target level of one or more measure that they intend to reach during a session or during training. The software may provide a means for the user to input a final rating or ongoing ratings of their experience during or following a session or training, for example pain, sadness, focus, anxiety, craving or other measures. For example, the software may provide the user with a UI slider, drop-down menu, text box, or other UI form elements. The software may also use automatic text scoring to score a user's text input, for example based upon AI means of heuristic categorization, counting key words, or otherwise assigning a quantitative level to text input. For example, if the user inputs text regarding their pain, the software may automatically score their text input for pain severity based upon the type and frequency of words or phrases found in the text that are associated with pain severity, or based on the computer-interpreted meaning. In addition, the process of assessing the user's text input may be completed or further facilitated by a person who may perform this process in all or in part. The software may determine and store differences between these measures. These differences may be used to present the user's progress, or to present their results relative to their target.

### Eye Movements, Voice Analysis, Facial Analysis

The software may track the user's eye position, eye movements, pupil dilation, perform voice analysis for content or emotional tone, and facial analysis for detecting emotion. Any of these may be used for determining the user's state, performance, mental or emotional results. The software may use a variety of means to track the user's attention level, or task performance. These may include eye tracking, use of performance of an alternate task or catch trials to determine a user's attention level or performance level or engagement level or focus level.

### Example Combination Methods

User's may be prescribed or recommended to use both the software provided and a specific pharmaceutical as a means of improving or treating a health condition or improving their health or wellness. When a user is provided with a prescription for a medication, the user may simultaneously or after receive a corresponding recommendation or prescription to use a particular stimulus, exercise or training regimen using the software provided.

In particular, conditions of psychology, psychiatry and the central nervous system, and pharmaceuticals engaging these, may be used in combination with software-based training to control related mental, cognitive or CNS functions, or related brain systems. For example, in combination with pharmaceutical treatment for depression, and user may be recommended to perform exercises guided by the provided software that are intended to decrease depression or increase control over depression.

Instructions

The software may provide stimuli or instructions to users to deliberately increase the efficacy or decrease the side-effects of a pharmaceutical or medication that they are taking, or in combination with a medical device, medical procedure or treatment. For example, if a user is receiving a pain medication such as an opioid, the user may receive instructions to practice a mental exercise of imagining the opioid working in the area of the user's body where they experience pain to decrease their pain. The user may be instructed to notice and/or note and/or measure any decreases or changes in pain that are brought about by the medication. Similar instructions may be used for other types and classes of pharmaceuticals. The user may be instructed to imagine the medication performing its known effects, and to attempt to generate greater effects. The user may be instructed to imagine the medication working in a part of the body where it is intended to work.

The software may provide stimuli or instructions to users to deliberately decrease side-effects of a pharmaceutical or medication that they are taking, or in combination with a medical device, medical procedure or treatment. For example, if a user is receiving a pain medication such as an opioid, the user may receive instructions to practice a mental exercise of decreasing nausea or opioid-related craving. The user may be instructed to notice and/or note and/or measure any decreases or changes in side-effects that are brought about by the medication. Similar instructions may be used for other types and classes of pharmaceuticals. The user may be instructed to imagine the medication showing decreased side effects. The user may be instructed to imagine decreased side effects in a part of the body where side effects may be observed.

### Synergistic Efficacy/Decreasing or Controlled Side Effects

Users may take a pharmaceutical and use the software in combination to increase the effect. In other words, the use of the software may increase the effect of the pharmaceutical, the use of the pharmaceutical may increase the effect of the software, or the two may have a synergistic effect. Specific combinations of stimuli, instructions or exercises and particular pharmaceuticals may be employed. For example, the software may select stimuli, content, instructions or exercises that are known or suspected to have synergistic effects with a particular pharmaceutical, pharmaceutical class, or pharmaceutical for a particular indication. For example, the software may select stimuli, instructions, exercises, or training related to pain reduction for use in combination with a medication used for pain reduction, such as gabapentin or an opioid. The software may select stimuli, instructions, exercises, or training related to depression for use in combination with a medication used for depression remediation, such as an SSRI or SNRI or antidepressant such as buproprion. The software may select stimuli, instructions, exercises, or training related to anxiety reduction or anxiety disorders including PTSD or OCD or phobias for use in combination with a medication used for anxiety reduction, such as a benzodiazepine such as valium. The software may select stimuli, instructions, exercises, or training related to addiction or craving reduction for use in combination with a medication used for addiction or craving reduction, such as methadone. The software may select stimuli, instructions, exercises, or training related to dieting or weight reduction for use in combination with a medication used for dieting or weight reduction, such as orlistat or belviq.

### Learned Placebo Effect, Learned Boosting of Therapeutic Effect

Users may take a pharmaceutical and use the software in combination to increase the ‘placebo’ effect or ‘nocebo’ effect. The placebo effect may be a psychological effect of a drug, or of a sham treatment, inactive treatment or ‘sugar pill’ that may produce or increase therapeutic efficacy or decrease side effects. The software provided may provide users with stimuli, instructions, or training that may increase the user's placebo effect. This may be used either with active medications or treatments, or it may be used with inactive medications or treatments, or treatments with unknown efficacy. The use of this software and method to boost the placebo effect may be accomplished with or without the user's knowledge. The software may indicate to the user that they will be learning to produce a placebo effect deliberately.

The software may teach the user specific strategies shown to increase or product the efficacy of a medication or treatment, real or sham. For example, the software may provide instructions for a user to imagine a treatment being highly efficacious. The software may provide instructions for a user to form a mental image of using or receiving any type of treatment. For example, the software may instruct the user to imagine putting a treatment cream on their body, or imagine taking a medication, or imagine the medication working within their body or on particular organs or systems or cells or receptors. The software may instruct the user to imagine receiving a treatment procedure from alternative health, or massage, or chiropractic care, or herbal remedy, or homeopathic remedy, or osteopathic care, or bodywork, or acupuncture, or biofeedback, or acupressure, or trigger point massage, or trigger point injection, or other injections, or electrical stimulation.

### With a Guide or Provider

The software may provide for interaction with a guide or provider, who may guide or make recommendations for the user, and receive corresponding information. For example, the guide may indicate or recommend what stimuli, exercises, training or content a user should receive. This recommendation may be based upon the characterization of the user provided by the software. The software may provide information to the provider regarding the user's progress, compliance with medication receipt or utilization or treatment compliance, for example based upon input from the user indicating their compliance, or based upon measures such as user health indicators (e.g. activity tracker shows exercise level or sleep level), or user location (e.g. GPS shows user has gone to a clinic), or interaction with other healthcare professionals. The software may provide information to the guide charting the user's progress, symptoms, usage levels. This information may be aggregated across users to indicate the overall level of success achieved by one or more methods or regimens. For example, if a guide recommends treatment for depression using a pharmaceutical plus a cognitive treatment for depression provided by the software, the guide may be provided a report of the time course of the user's symptoms, for example their BDI score. The guide may be provided receive aggregate information for multiple users that they have recommended this treatment regimen for. The guide may be provided information for multiple users from multiple guides or providers or physicians. The results from different treatment regimens may also be provided for comparison. For example, the software may provide a graph of an individual user's progress or a group of user's aggregate progress vs. another group of users, or another group of users receiving a different treatment regimen. For example, the software may provide a graph of the pain level of a user receiving treatment with (or without) a pain medication and with (or without) a training regimen for pain provided by the software, and also a graph of average response of a prior group of users who received similar treatment and/or training. The software may compute the response of a user as a percentile rank comparing their results with those observed in prior user groups.

### Target Brain State Training

The methods, devices, software and systems provided herein may be used to perform target brain state training where a user is trained to achieve a selected target brain state of activation. A target brain state of activation may be a spatial activity pattern within a region of the brain, a series of regions of the brain, or the entire brain. The user may be trained using stimuli, instructions, or exercises previously demonstrated to produce a target brain state. For example, if users have been tested using a set of instructions and it has been demonstrated using fMRI or brain imaging that this set of instructions leads the users to produce a particular pattern of brain activation that is desirable for a given purpose, this set of instructions may be provided to future users in order to produce similar brain states or patterns of brain activation.

### High Performance or High Motivation State Training

The methods, devices, software and systems provided herein may also be used to determine which types of physiological activity patterns correlate with certain types of desirable cognitive or behavioral processes, such as high performance states or ‘flow’ states, and then to train users to create those activity patterns.

### Selecting Tasks and Training to Appropriate Level of Challenge

The methods, devices, software and systems provided herein may also be used to set appropriate levels of challenge for tasks that are to be undertaken by users either inside or outside of the measurement of physiological information, based upon the patterns of physiological activation that are evoked by those tasks during measurement. When a user fails to be able to correctly perform a task, such as a sensory perception, motor act, or cognitive process, activity patterns are measurably different than in the condition when the user does correctly perform the task. Therefore, this method includes measuring the average pattern of activity for more than one level of task difficulty, optionally determining a threshold level of task difficulty that leads to a defined level of activity, and then selecting tasks for the user at a level of difficulty corresponding to a particular measured level of activity, such as a level above, at, or below the determined threshold. For each level of task difficulty, the average pattern of activity may be determined. A threshold may then be selected as a level of task difficulty that leads to a particular level of activity, or a particular percent of trials where an activity metric reaches a criterion level. With this information, it is possible to adjust task difficulty or rate to be at or near the threshold of the user's ability to achieve a given physiological response and to correctly perform the task.

### Behavior, Movement, Rehabilitative, Performance and Sports Training

Sports and performance training may be facilitated using the methods of the methods, devices, software and systems provided herein. It is known that practice, as well as mental rehearsal in the absence of actual activity, can improve performance in a variety of tasks and activities. Training according to the methods, devices, software and systems provided herein may be used to guide the practice or mental rehearsal of an activity in order to produce faster and more effective learning than practice or mental rehearsal would achieve without such assistance.

For example, the behavior employed in training may be a mental rehearsal, such as a musician rehearsing a piece of music. In such case, the musician might be shown music and mentally envision himself conducting. The musician can learn to achieve a higher level of brain activity when practicing. Achieving a higher level of brain activity may enhance the effectiveness of such practice.

### Training Users to Become Increasingly Aware of Spatial Activity Patterns

The methods, devices, software and systems provided herein may also be used to train users to become increasingly aware of the presence or absence of particular patterns of activation in their brain, such as activity levels or spatial activity patterns, as observed using introspection by the user of their own experiential states. By training users to be aware of the presence of experiential components associated with a particular mental state or performance state, users may make improved judgments of when to engage in particular behaviors outside of the presence of measurement equipment.

Use in Combination with Other Interventions

The methods described in the methods, devices, software and systems provided herein may be used in combination with a number of different additional methods, as described here.

Combination with Additional Therapies and Methods

The methods, devices, software and systems provided herein can be used in combination with a variety of additional and non-traditional therapies and methods including: rehabilitative massage, sports or other massage, guided visualization, meditation, biofeedback, hypnosis, relaxation techniques, acupressure, acupuncture. In each case, the user can undergo the non-traditional therapy technique while undergoing training. The non-traditional therapy technique can be used to enhance the users ability to succeed at training to control and exercise a given brain region. In addition, the training methodology can allow for improved outcomes based upon the use of these non-traditional therapeutic techniques.

Combination with Physical Therapy

The methods, devices, software and systems provided herein can be performed in combination with physical therapy. In such case, the training may be prescribed in combination with physical therapy. The methods, devices, software and systems provided herein may be used to speed the improvement produced by the exercises of physical therapy. The methods, devices, software and systems provided herein may also be used to measure the improvement or change in functioning produced by physical therapy over the course of treatment. In addition, the user can undergo physical therapy exercises as an adjunct to the use of this method.

Combination with Psychological Counseling or Psychotherapy

The methods, devices, software and systems provided herein can be combined with psychological counseling or psychotherapy. The user can undergo interchange with a psychological counselor or psychotherapist while undergoing measurement and training as described in the methods, devices, software and systems provided herein to evaluate the person's response. For example, therapy may relate to stress or anger management where how effectively stress or anger is being managed is measured during therapy. The user can also undergo psychological counseling or psychotherapy as an adjunct to the use of this method. The therapist or counselor may provide methods, devices, software and systems provided herein to be used as ‘homework’ for the user to complete on their own, either during or between sessions.

### Software Feature Overview Related to Substance Use Disorder

The software may provide any of the following features related to substance use disorder.

Provide users and clinicians with validated assessment tests to assess SUD risk within a mobile app

Based on validated assessment results, use sophisticated Bayesian statistical inference to select recommended items for an individually-tailored treatment plan

Allow PCP's along with users, user's support system and other providers to create/select/adjust an individually-tailored treatment plan through all phases of treatment

Provide psycho-education to increase motivation, commitment and adherence to treatment plan items

Support coordination of care and linkage with indicated follow-up treatment providers as needed

Provide scheduled, real time, multimedia reminders via mobile, web, email, SMS

Provide scheduled or randomly-presented, real time ecological momentary assessments via mobile

Allow different parties (user, PCP, user's support system and relevant providers) a means to easily record and view user adherence with different plan elements using an integrated platform

Use device technology (GIS/GPS) to verify user location/adherence (e.g. verify being at meeting)

Provide a user-specific progress/adherence dashboard, accessible to the PCP or members of user's support group (as appropriate, to people who have received a user-specific password or access)

Provide incentives such as medallions, badges, scores, which can be tied to monetary rewards if desired

Provide user with validated multimedia feedback regarding treatment plan adherence

Provide the treatment plan option of multimedia training in cognitive self-management relevant for SUD (CBT-based)

Provide treatment plan option of multimedia training managing substance-related craving

### Software Platform and App: Example Elements of Features and Design

The platform may provide mobile/web-based technology that monitors and guides users through a treatment plan including a broad variety of highly-optimized cognitive strategies, including CBT-like exercises, guided visualizations, reframing exercises, attention control exercises and many others. Users login via web browser or mobile/tablet device and complete sessions multiple times per week. User adherence and progress may be tracked in detail. This approach allows highly uniform, broad deployment and testing of cognitive therapeutic approaches with detailed user tracking. The existing platform may be adapted to SUD treatment.

### User Characterization

Users may provide comprehensive information using validated assessment tools such as the DAST-10 and CAGE-AID regarding their risk level for SUD, their health and cognitive strategies they may employ, and other aspects of their personality and condition. All user information may be transferred/maintained securely and may be ‘anonymized’ on the server for full HIPAA compliance.

### Treatment Plan Creation

The software may pre-select recommended treatment plan elements based upon a Bayesian inference engine using the data from the user's characterization. The user and PCP in collaboration may then select these or additional treatment plan elements (e.g. indicated medication, linkage to appropriate follow-up treatment provider, urine test, 12 step meeting), or create custom elements for the user (e.g. talk to your sponsor Steve, go bicycle riding for exercise).

### Online Training

Users may use the mobile software to follow an individually-pre-selected treatment plan. This treatment plan may include a sequence of cognitive training or other exercises. Each strategy may be explained and depicted in audio/video, and the users may provide continuous user engagement, ratings, and feedback. The strategies for SUD may increase users' awareness and control over craving, motivation, and SUD-related decision-making. They may be similar in their intent to many interventions used by clinicians, such as CBT or a motivational interview. As the user proceeds through the training exercises, the tracking features may monitor their progress on a day-by-day and trial-by-trial basis, providing ongoing encouragement, rewards, and positive feedback.

### CBT-Based Multimedia Modules

The software may include mobile/web-based deployment of validated cognitive behavioral therapy (CBT) treatments as feedback to users that can be deployed digitally. CBT is a validated treatment for SUD, and has been reported to be effective. The CBT program may provide separate modules for a) functional analysis and b) skills training.

### Craving-Control Multimedia Modules for SUD

The software may also include mobile/web-based deployment of strategies for learning control over substance-related craving. Examples of cognitive strategies to decrease craving may include cognitive reframing, focusing intention and perception on experiences when users have less or no desire to use, visualizing positive alternatives to using, visualizations of detailed scenarios as a non-user, visualizing the negative health consequences of using. These may be available if selected to be part of a treatment plan by the PCP in collaboration with the user.

### Continuous Innovation of Uniformly Deployed Treatment Plan Elements

One of the most exciting features of the software platform is its ability to use quantitative Bayesian inference or other inference methods to track and test the success of each of the treatment plan elements, or subtle variants of them, based on both efficacy and user preference. The software may continuously test the success of each existing treatment plan element, instruction, stimulus, or strategy across all users using it (or by user sub-group), based on a variety of quantitative metrics including user reactions and outcomes measures using validated instruments. This may allow for a process akin to adaptation and natural selection: stimuli, instructions, treatment plan elements and strategies may be adapted, modified, refined, scored, and then selected based upon user adherence levels and user outcomes. The interface may collect users' and/or guides' suggestions about creating new treatment plan elements or strategies or modifying existing ones, so thousands of peoples' creative input may be captured. This may allow continuous innovation, testing, quantitative selection, and improvement. The highly-tested methods developed in this way, for examples methods for cognitive therapy or user training or instruction, may be used within the app, and may be provide for use in other treatment contexts as well, such as in traditional one-on-one clinician/user settings or therapy.

The software here may continuously test new strategies on large volumes of users allowing rapid selection and deployment of novel or optimized approaches. With each release of the technology the strategies may be improved over the last release, and deployed in real time to existing users.

### Software Feature Examples

### Treatment Plan Recommendation Engine

The software platform may provide a Bayesian or other inference engine to recommend and ‘pre-select’ the elements of a treatment or stimulus or instruction program for a user based with the highest likelihood of success based upon the characterization, risk level and other factors from the user's assessments. These recommended elements may be ‘checked’ in a checklist of treatment plan elements that can then be modified, or can be customized by creating additional, personalized elements. The selection of treatment plan elements may take place involving both the user and the PCP, guide or members of user's support system.

### Treatment Plan Creation

The software may provide a common platform for a guide and/or a user (and/or the user's support system or follow-up providers where appropriate) to create a patient treatment plan based upon the recommendations made by the software, and based upon individual-appropriate choices. After patient assessment and treatment plan recommendations are provided by the software, the guide and patient may select, adjust and discuss the treatment plan recommendations provided by the software. The treatment plan may be individualized by creating personalized items (e.g. entering a new text item: ‘Avoid being at . . . , Avoid interactions with . . . ’).

### Scheduling of Treatment Plan Items

The software implementation of the treatment plan may also allow scheduling of when each plan item is to be completed by the user on a daily, weekly, or monthly basis, allowing scheduling by day or by time.

### Treatment Plan and Adherence Monitoring and Rating by Guide, Patient/User

The software platform, viewable either within a web browser, tablet, or mobile device, may have an individual dashboard for each patient/user. This dashboard may be menu accessible by the patient, the guide/PCP/caregiver, and any members of the patient's support system invited by the patient and/or guide to create individual logins with access to the patient's account. Each of these people may have their own login/password, and each may have individualized authorization to access the patient's status information.

The software dashboard may display overall usage statistics and treatment plan adherence for the user/patient, for example displaying percent of treatment plan items completed, patient ratings for items, or decrease in substance use if appropriate. The dashboard may also display daily, weekly, and monthly view of which treatment plan items were and were not completed.

### Automated Treatment Plan Monitoring

The software platform may also have the ability to perform optional continuous, automated treatment plan monitoring. The platform can send out alerts based upon treatment plan adherence, or lack of adherence.

### Treatment Plan Feedback

After the creation of a treatment plan for a patient, the software may provide friendly customized multimedia content for timely delivery to the patient for the purpose of encouraging the patient to maintain adherence, assimilate behavioral strategies, and develop cognitive control over craving. User-friendly feedback may be tailored to match the patient's risk level. Feedback may progress as assessed by actions taken by patient and patient's self-assessment on the stages of change. See wireframes below.

### Continuous Optimization of Feedback Content

The software platform may have the capability to optimize chosen treatment plan elements over time based upon subject ratings, success, and usage. For example, for subjects who are receiving multimedia cognitive strategy training exercises within the software, if selected, the software platform may individually tailor the content being provided in real time based upon which strategies lead to the greatest observed decreases in the user's substance-related-craving, are found most helpful by user, etc. This can take place down to the level of individual cognitive training instructions (e.g. 1-30 s long content elements).

The information gathered regarding patient adherence, outcomes, and preference may be aggregated across numbers of users so that the Bayesian ‘prior’ that is the basis for treatment plan item recommendations for users may reflects a growing database maintained by the software of success across users. The information may also be patient-specific, and the Bayesian ‘prior’ upon which recommendations are made or content is selected for a subject by the software may reflect the characteristics, risk level, and success of each individual user up to that time.

### User Feedback

Feedback may be designed to increase the patient's motivation and commitment to self-management and to health promoting behaviors. Cognitive training strategies provided by the software may be comprised of standard CBT strategies or other strategies that are suitable. The software may continuously measure the effectiveness of each strategy, so over time the most effective strategies based upon user outcomes may be selected (the Bayesian prior used in selection of treatment plan items for recommendation may reflect this).

### Research Test Bed for Extending Validation of Therapeutic Methods

This platform may create a large test-bed for research extending the existing evidence base regarding consistently-deployed behavioral therapy elements. Given the large anticipated patient population and extensive data gathering, the software may accurately and quantitatively determine the usage and efficacy statistics for many different self-management skills, strategies, stimuli, instructions, and treatment plan elements.

The dashboard provided by the software may make it possible for decisions to be made jointly between the user and guide or provider, and/or social network, and may make clear which items the patient has been adhering to, which ones the patient has found helpful, and what their rates of utilization are, making it possible to review and update the treatment plan on an ongoing basis.

The software platform may provide cross-platform, secure tools to function equivalently and at high performance in a desktop/browser based context or on a mobile/smartphone/tablet device.

Software access may be available via desktop, tablet, and phone to the patient, guide/PCP, and support networks where appropriate. The software may operate cross-platform so that when wearable mobile devices such as the Apple Watch and others become ubiquitous, the software may be deployed there as well.

The software platform may be linked to API hooks of EMR/EHR systems, providing the ability to import data into personal health records (PHRs) that provide standards-compliant APIs. The software may integrate with EMR/EHRs to exchange information, providing patient data to the EHR, or accessing patient information from an EHR. This may allow guides/providers and patients to view and track their software-generated data in the context of their other health information, using any features provided by the PHR, and providing greater linkage between healthcare providers in the context of treatment.

The software may track patient usage and completion of treatment plan objectives in detail. The software may award different medallions for meeting specific goals. These may be used in conjunction with patient-familiar 12-step goals where appropriate (for example awarding of medallions based on days/months/years of sobriety). Medallions may also be tied to the successful accomplishment of other treatment plan objectives (e.g. number of days that all treatment plan objectives were met, number of cognitive training modules completed). In addition, medallions may be used that tie to monetary rewards that may be provided to the subject (e.g. $1, $5, $10, etc. medallions, and a scoring system for accumulating them).

Patient and guide/PCP may select days/times for scheduling reminders on a software UI. The reminders may be delivered by the software by email/text message or recorded audio/voice message.

The software may include a ‘resources’ page appropriate to the patient's risk level, as well as social networking resource links. The software may provide PCP with a search engine page to identify appropriate local resources.

The software may provide a user map of other users and/or users who have registered as guides or providers and support groups, allowing PCPs to find providers already using the software in their area, and allowing providers and support groups to offer their services to users of the app.

Server-based data may be anonymized and secure. Users may use secure and encrypted login procedures provided by the software.

The software may allow for easy creation of ecological momentary patient assessments (e.g. level of substance craving, level of temptation provided by the environment, mood, anxiety).

Assessments may be sent out to a user via the software platform, and may be responded to quickly by patients through single-click choice selections. The software may store the user's selections, time and the geographic location where the EMA was made based upon device hardware. For example, users may initiate an assessment when they engage in substance use. In parallel, patients are prompted at random or pre-scheduled times to complete assessments when not using drugs.

The software platform may provides for the use of most of mobile device technologies.

### GIS/GPS.

Each use of the software may stored along with time and location information, allowing verification of treatment adherence. For example, if a treatment plan element indicates that the user should attend a session with a health care provider, attend a 12-step meeting, or go somewhere to exercise, then the user's check-in that they accomplished this task may be accompanied with geographic location information that verifies when and where they did so.

### Bluetooth.

The software may use built-in communication functionality of devices (including WiFi, Bluetooth, Cellular, etc.) for whatever functions require it.

### Real-Time Video Communication.

The software may allow videoconferencing, for example between user/patient and guide/PCP, or for group videoconferences.

Example Protocol/Sequence of Events within Software for Patient Treatment:
1) A primary care provider with extensive experience characterizing patients at high risk may help to recruit and evaluate patients in detail in person.
2) Patients may perform assessment tests using the software.
3) The software may recommend an individualized treatment plan for each patient using its Bayesian inference engine to suggest the treatment plan options that may be most likely to be useful for the patient based on risk level and other factors.
4) The guide/PCP and user/patient may create a customize treatment plan by accepting or rejecting the recommended treatment plan items, or other selectable treatment plan items. They may also create free-form individualized treatment plan items specific to the patient.
5) The patient, PCP, other care providers, and members of the patient's support network may be invited by the patient through the software to have separate logins/passwords that allow private, HIPAA-compliant access to the patient's information.
6) The user/patient may use the software, and may receive multimedia treatment plan reminders and feedback.
7) The user/patient or guide/providers may check off completed treatment plan items within the software UI. Some items the patient may check off, some items may be checked off by others (such as providers, support network members, sponsors) to support adherence.
8) The patient may regularly receive ecological momentary assessment questionnaires throughout the period, provided by the software.
9) If selected as part of the treatment plan, the user/patient may receive multimedia-based cognitive training, such as CBT or the Brainful suite of cognitive training exercises designed to decrease craving.
10) The patient, PCP, and invited members of the patient's support network may have HIPAA-compliant access to the patient's individual dashboard to observe the patient's progress, treatment plan adherence, accomplishments/medallions within their plan.
11) Following completion of this protocol, the PCP, patient, and members of the support network as appropriate may be interviewed in detail, and may fill out detailed questionnaires and survey instruments to assess the usability of the software, and to determine points for improvement, which could then be made.

### Example Software Features

Scheduling. Scheduling of software reminders to help with user adherence to treatment plan. This may allow automatic user notifications, which may potentially be sent via email, sms, push notification, telephone audio, etc.

Rating. Symptom severity 0-100 may be gathered/stored at beginning and end of each session, for example craving or pain level. This allows detailed tracking of user status and how it has changed.

Control. Audio, text, image or video content leading user through multi-media feedback and training content may be provided by the software. The software may provide a suite of cognitive training exercises. The platform may make it possible to add additional modules, and additional content may be added. Cognitive exercises may be designed to engage neuroplasticity through repeated exercise of desired neural activation, such as practice at decreasing substance-related craving, visualizing negative life-impacts of substance use, thinking through positive alternatives to challenging situations, etc. Following each instruction, users may have a period of time (length automatically adjusted to user level) to practice each instruction, leading to greater ability, and to neuroplasticity.

Awareness. Software may provide an interface for users to rate their reaction to each individual action or instruction, for example the change in their level of craving. This may allow tailoring of instructions to the user, and also may allow for gathering population data for continuous improvement of instructions.

Background Content. The software may provide user-selectable background video, audio content—e.g. relaxing sounds and video.

User Status. The software may provide motivating information about the user's progress, including usage statistics, symptom severity changes, preferred exercises, accomplishment of goals, etc.

### Creation, Selection, and Optimization of Novel Cognitive Strategies

Mental exercises or strategies may be known to engage desired brain circuitry or neurochemistry and/or produce desired behavioral effects. If users are provided these exercises by the software and practice these strategies, then through a combination of practice effects and neuroplasticity, they may improve in their ability to perform the strategies, and produce activation of corresponding brain areas.

Cognitive strategies may be developed and optimized for a purpose such as control over pain or substance-related craving through a process of continuous selection that is analogous to natural selection: Find existing cognitive strategies, create new strategies and adapt existing ones by making changes. Compete these strategies against each other in extensive subject testing. Measure the impact of trials of each strategy based upon brain activation and/or behavioral measures. Select optimal strategies that produce the biggest impact (brain activation or behavioral change). Continue this process to further optimize strategies.

Development of Cognitive Training Exercises Through Real Time Brain Imaging, Traditional Approaches

A number of existing cognitive strategies derived from CBT, motivational interviewing, relaxation techniques, guided visualization, and other established methods may serve as the starting point for a development process involving providing these strategies by software during real time fMRI brain scans or other physiological measurements in subjects learning cognitive strategies during measurement, for example inside of a 3.0 Tesla fMRI scanner. At-home sessions may also be provided by software using similar strategies presented via mobile/web-based devices. The exercises may be individually developed, tested, and optimized using computerized presentation and a combination of real time neuroimaging or physiological measurement, real time quantitative subject ratings, and qualitative feedback and suggestions for improvements. Each of the individual trials of each exercise may be scored, using either subject ratings and/or fMRI brain activation metrics based upon their ability to activate targeted brain systems. Cognitive strategies may be ‘competed’ with other strategies, using a points system, for example, with the victorious strategies moving forward into further testing and refinement. Using this process, the strategies may evolve through successive generations, and may be highly selected and optimized.

Continuous Selection, Testing and Strategy Improvement Platform for at-Home Testing

The software platform may continue this process of quantitative testing, selection, and competitive refinement of these cognitive strategies, even in the absence of physiological measurement or brain scanning. The software may track user activities and responses in intimate detail in real time, and may use Bayesian or other inference methods to individually-select the sequence of instructions presented to each user to optimize user outcomes. The software may record user data such as response to individual instructions (even down to the level of seconds), and may highly optimize what instructions each user receives, and also may continuously compare and improve effectiveness of different instructions in this way. Even minor variants of different cognitive instructions may be compared over trials, which may lead to statistically-relevant comparisons of effectiveness. This may be used down the level of single word changes within instructions. Effectiveness of cognitive strategies may be compared, for example by software, based upon measures of user satisfaction, changes in user sensations such as pain or craving or mood, or based on long-term outcomes measures using validated instruments at later time points (e.g. BDI, MPQ, COMM). This continuous improvement platform may continue to lead to greater and greater effectiveness in cognitive training exercises, and ability to rapidly test existing or new approaches. This process of analysis may be performed in a fashion that involves both software analysis, and human selection based upon results. For example, a person may view the analyzed data for which strategies have been most effective for a given condition, and select those strategies for input into the software for use in future users.

Cognitive strategies may be tested and scored in this fashion by software or by investigators, either inside of an fMRI scanner or using web or mobile-deployed or at-home training. For example, when patients use strategies that altered pain perception during fMRI, significant brain activation changes may be measured in many pain-related regions associated with pain (FDR>0.05). Different classes of strategies may be associated with different patterns of brain activation when the strategy epochs are contrasted with baseline or with each other by t-test. For example: brain areas activated during sensory strategies may include bilateral SI, SII, and dorsal ACC; brain areas activated during affective strategies may include right anterior insular cortex. FMRI activation measures may be made for each strategy in multiple regions of interest, allowing quantitative comparison of each strategy in each system. In this way, it is possible to determine strategies that may be effective in activating or deactivating particular brain systems. It is possible to determine strategies that may be effective in activating or deactivating particular patterns of brain activation, for example by comparing brain activation patterns on a region-by-region or voxel-by-voxel basis.

Brain Imaging

The brain is the seat of psychological, cognitive, emotional, sensory and motoric activities. By its control, each of these elements may be controlled as well. The present methods, devices, software and systems provided herein may be used to provide and enhance the activation and control of one or more regions of interest, particularly through training and exercising those regions of interest. An overview diagram depicting the components and process of the methods, devices, software and systems provided herein is presented in FIG. 1.

### Further Example Embodiments

One particular aspect of the methods, devices, software and systems provided herein relates to systems that may be used in combination with performing the various methods according to the present methods, devices, software and systems provided herein. These systems may include a brain activity measurement apparatus, such as a magnetic resonance imaging scanner, one or more processors and software according to the present methods, devices, software and systems provided herein. These systems may also include mechanisms for communicating information such as instructions, stimulus information, physiological measurement related information, and/or user performance related information to the user or an operator. Such communication mechanisms may include a display, for example a display adapted to be viewable by the user while brain activity measurements are being taken. The communication mechanisms may also include mechanisms for delivering audio, tactile, temperature, or proprioceptive information to the user. In some instances, the systems further include a mechanism by which the user may input information to the system, preferably while brain activity measurements are being taken. Such communication mechanisms may include remote delivery such as delivery via the internet or world wide web, or delivery using wired or wireless transmission to a mobile phone, tablet, or desktop-based web browser or downloadable software.

In one embodiment, a method is provided for selecting how to achieve activation of one or more regions of interest of a user or change one or more symptoms, the method comprising: evaluating a set of behaviors that a user separately performs regarding how well each of the behaviors in the set activate the one or more regions of interest or change one or more symptoms; and selecting a subset of the behaviors from the set found to be effective in activating the one or more regions of interest or one or more symptoms. In one variation, evaluating the set of behaviors comprises calculating and comparing activation metrics computed for each behavior based on measured activities for the different behaviors. In one variation, the behaviors evaluated are overt behaviors involving a physical motion of the body of the user. In another variation, the behaviors are covert behaviors only cognitive processes which do not lead to a physical motion of the body of the user.

Also according to any of the above embodiments, the behavior may optionally be selected from the group consisting of sensory perceptions, detection or discrimination, motor activities, cognitive processes, emotional tasks, and verbal tasks.

Also according to any of the above embodiments, the methods are optionally performed with the measurement apparatus remaining about the user during the method. According to any of the above embodiments, in one variation, measuring activation is performed by fMRI.

According to any of the above embodiments, in one variation, the activity measurements are made using an apparatus capable of taking measurements from one or more internal voxels without substantial contamination of the measurements by activity from regions intervening between the internal voxels being measured and where the measurement apparatus collects the data. Also according to any of the above embodiments, pretraining is optionally performed as part of the method.

Determining a Treatment Method for a Given Condition

This section describes a process by which treatment methods for different conditions may be developed. It is noted that the users referred to in this section are not necessarily users that are being treated according to the present methods, devices, software and systems provided herein. Instead, the users referred to in this section are people who are used to evaluate how well given stimuli, instructions for behaviors activate certain brain regions.

Developing treatment methods for different conditions may be performed by evaluating a likely effectiveness of treating a given condition by understanding whether there is an association between a given condition and a particular training regimen; determining the one or more regions or cognitive or mental processes of interest to be trained for the given condition; determining one or more classes of exercises likely to engage those brain regions or cognitive or mental processes; determining a set of exemplar exercises from the one or more classes for use in training; and testing the user to ensure that the set of exemplar exercises are effective in activating the regions of interest or cognitive or mental processes.

Evaluating Likely Effectiveness of Treating a Given Condition

Numerous different conditions may benefit from training according to the present methods, devices, software and systems provided herein. The likelihood of success for a given condition to be treated according to the present methods, devices, software and systems provided herein may be evaluated from knowledge of the etiology and variety of causal factors contributing to the condition as understood at the time of treatment. More specifically, when considering whether treatment may be effective for a given condition, attention may be given to whether the condition is related to brain activity. If there is a correlation between the presence of the condition and a level or pattern of brain activity in one or more regions of interest, then, the methods of the present methods, devices, software and systems provided herein may improve that condition by altering the level or pattern of brain activity in the one or more particular brain regions or cognitive or mental processes. Following use in significant numbers of people, statistical inference may be used to determine which conditions may be best treated using this method, and which exercises, instructions, postures etc may be most effective for any condition.

Different regions of the brain may be associated with different functions, different conditions and mental states, and may thereby be engaged and exercised by particular types of stimuli, or by particular behaviors associated with those functions. Hence, by understanding what function a given region of the brain performs, exercises may be designed which activate those brain regions. Through trial and error, exercises may be varied and thereby fine tuned both with regard to their effectiveness in general, and with regard to their effectiveness for a given user.

Once a general class of exercises has been determined for a given mental state, actual instances of specific stimuli or behaviors may be created that are able to improve that mental state. The stimuli or instructions for behaviors to be used may be created from within the class of stimuli or instructions for behaviors that may engage the mental state or brain region of interest. The exemplars created may be real stimuli that may be presented to users, or real instructions that may lead the user to engage in behaviors. These stimuli and instructions may be created via computer to be presented digitally. Instructions may include instructions that will inform the user of what to do and be presented either on the monitor, or they may include verbal instructions presented via digital audio, or the instructions can include icons or movies presented to the user.

In many instances, the process of creating stimuli or instructions for behaviors may be iterative, with the initial stimuli or instructions for behaviors created being fine-tuned. This may be performed by first determining the appropriateness of the stimuli or instructions for behaviors by testing them in users. It is noted that this is may be an objective evaluation of the effectiveness of the behavioral instructions or stimuli. This evaluation may be used for the subject(s) with which it was determined, or for other subject(s).

Stimuli or instructions for behaviors may be presented by software in the context of a psychophysically controlled task or measurement or an operant conditioning task, or a computer game or other contexts. The user may be asked to detect the stimuli or make discriminations among them when they are presented using computer-controlled software, or asked to perform the behaviors. This may allow the stimuli or instructions for behaviors to be optimized to be close to the user's behavioral ability threshold, or ability to detect or make discriminations among them. Stimuli may be selected that are slightly harder than the user can achieve, similar to what the user can achieve, and easier than what the user can achieve.

Defining User Selection Criteria and Screening Users

It may be desirable for the treatments of the present methods, devices, software and systems provided herein to have a high frequency of success. It may therefore be desirable to select users based upon the likelihood of their treatment, training or use of the software/device being successful. Examples of selection criteria that may be used include but are not limited to: 1) Whether the user has the condition for which treatment is intended, based upon diagnostic criteria. 2) Whether the user has other, preferable treatment options available. 3) Whether the user has sufficient cognitive ability to participate in training. 4) Any indicators predictive of treatment success, such as previous success or failure of the method with users that are similar based upon diagnostic group or other signs and symptoms. Each potential user may be screened based upon some or all of these selection criteria to determine their suitability for training.

Measuring and Displaying of Physiological Activity

Substantially throughout the process of training, the physiology of the user may be measured. This information may be presented to the user and/or the guide and/or device operator, and may also be used for additional computations such as the computation of metrics from a brain or body region of interest. This process may take place at a regular repetition rate, such as one set of measurements per second in one example, or at an alternate sampling rate.

User's Decreasing Need for Training

In general, the improvements that users are trained on through the use of the methods, devices, software and systems provided herein may be enduring outside of the context of training. Increases in performance or in the strength of activation of neural areas may be thought of as being analogous to the increase in muscle strength achieve through weight lifting, which persists outside of the context of the weight-training facility. Eventually, the user may come to be able to control their mental or physiological state without access to training provided by the methods, devices, software and systems provided herein at all, and/or may undergo ongoing improvements or decreases in symptoms. Therefore, the user's schedule of training or use may be tapered, or training or use may be discontinued when the user achieves a target level.

Performing Training Exercises in the Absence of Measurement or the Device/Software

An aspect of the methods, devices, software and systems provided herein relates to a further user performing training that is effective in regulating physiological activity in one or more regions of interest of that user's brain or a mental exercise or experiencing stimuli or content in the absence of information regarding the user's brain states or performance. Once stimuli, content, or instructions have been selected using the methods provided, and/or a user has been trained in controlling an activity metric in a region of interest with the presence of information about this activity metric, the users may be trained to continue to achieve this control and exercise of the corresponding brain regions in the absence of substantially real time information regarding the activity metric. This training may take place using training software largely analogous to that used inside a training apparatus, but run on a different device. This device may be independent of physiological or other measurement apparatus. In place of measurement information, the software may either use simulated information, such as random information, or it may use information from the same user collected during measurement, or it may use no information at all and omit presentation, or it may use information provided by the user, including the user's self-assessment of internal mental or cognitive states.

### General Examples

In a general aspect, a computer-implemented method of directing mental exercise includes providing, by a first output component of a computing device, a stimulus representing an imagined perception, experience or activity that a user should attempt to generate in their mind. The method also includes providing, by a second output component of the computing device, an instruction for the user to perform a mental exercise comprising instructing the user to generate an internal felt sense of the imagined perception, experience or activity. The method further includes providing, on a display screen of the computing device, a moving object, and wherein the instruction for the user to perform the mental exercise instructs the user to provide an input that characterizes the user's internal felt sense based in part on the motion of the object. The method further includes receiving, at a user interface of the computing device, the input that characterizes the user's internal felt sense, the input comprising an overt response from the user. The method further includes determining, by a processing module of the computing device, an attribute of the received input, and determining, by the processing module of the computing device and based on the determined attribute, a next instruction. The method further includes storing at least one of the determined attribute and the determined next instruction in one or more memory locations of the computing device. The method further includes training the user, including: (i) presenting the determined attribute, and (ii) providing, by the second output component, the next instruction.

In another general aspect, a computer-implemented method of directing mental exercise, includes providing, by a first output component of a computing device, a stimulus representing an imagined perception, experience or activity that a user should attempt to generate in their mind. The method also includes providing, by a second output component of the computing device, an instruction for the user to perform a mental exercise comprising instructing the user to generate an internal felt sense of the imagined perception, experience or activity. The method further includes receiving, at a user interface of the computing device, an input that characterizes the user's internal felt sense, the input comprising an overt response from the user. The method further includes determining, by a processing module of the computing device, an attribute of the received input, and determining, by the processing module of the computing device and based on the determined attribute, a next instruction. The method further includes storing at least one of the determined attribute and the determined next instruction in one or more memory locations of the computing device. The method further includes training the user, including: (i) presenting the determined attribute, and (ii) providing, by the second output component, the next instruction. The imagined perception, experience or activity includes a first aspect and a second aspect, and wherein the instruction for the user to perform a mental exercise includes a first instruction to generate the first aspect of the internal felt sense of the imagined perception, experience or activity, and also includes a second instruction to generate the second aspect of the internal felt sense of the imagined perception, experience or activity.

In yet another general aspect, a computer-implemented method of directing mental exercise includes providing, by a first output component of a computing device, a stimulus representing an imagined perception, experience or activity that a user should attempt to generate in their mind. The method also includes providing, by a second output component of the computing device, an instruction for the user to perform a mental rehearsal comprising instructing the user to generate an internal felt sense of the imagined perception, experience or activity. The method further includes providing, on a display screen of the computing device, a moving object, wherein motion of the object is configured to guide timing of the mental rehearsal. The method further includes receiving, at a user interface of the computing device, the input that characterizes the user's internal felt sense, the input comprising an overt response from the user. The method further includes determining, by a processing module of the computing device, an attribute of the received input, and determining, by the processing module of the computing device and based on the determined attribute, a next instruction. The method further includes storing at least one of the determined attribute and the determined next instruction in one or more memory locations of the computing device. The method further includes training the user, including: (i) presenting the determined attribute, and (ii) providing, by the second output component, the next instruction.

Various implementations may include one or more of the following. The stimulus may include an image. The stimulus may include a video. The stimulus may include a sound. The stimulus may include an animation. The stimulus may include a scent. The stimulus may include a tactile stimulus. The input that characterizes the user's internal felt sense may characterize a vividness of the user's internal felt sense. The input that characterizes the user's internal felt sense may characterize a specificity of the user's internal felt sense. The input that characterizes the user's internal felt sense may characterize an imagined physical extent of the user's internal felt sense. The input that characterizes the user's internal felt sense may characterize a quality of the user's internal felt sense. The input that characterizes the user's internal felt sense may be a subjective assessment by the user of whether the exercise was a success. The first output component may be different from the second output component. The first output component may be the same as the second output component. The method may be used with a medication therapy that includes a medication, and the method may further include providing an instruction for the user regarding the medication. The instruction for the user regarding the medication may include a reminder. The instruction for the user regarding the medication may include a dosage recommendation. The method may further include transmitting a message that includes an indication of the medication and of a performance of the user. The message may be transmitted for receipt by a computing device associated with a practitioner. The message may provide a dosage recommendation for the medication based on a performance of the user. The method may further include receiving a second message from the computing device associated with the practitioner, where the second message includes a change in dosage for the medication, and the method may further include communicating the change in dosage for the medication to the user. The method may be used with a physical therapy. The mental exercise may have an internal, covert proximate cause. The mental exercise may produce an internal, covert proximal result. The internal, covert proximal result may be a change in the internal felt sense of the user. The method may not include use of a biofeedback or physiological measurement device. The user's internal felt sense may include an internal subjective experience. The first instruction may be to imagine a sensation of warmth, and the second instruction may be to imagine a sensation of coldness. The method of directing mental exercise may be used to decrease pain. The method of directing mental exercise may be used to decrease stress. The method of directing mental exercise may be used to treat depression. The method of directing mental exercise may be used to treat anxiety. The method of directing mental exercise may be used to treat addiction. The method of directing mental exercise may be used to decrease craving. The method of directing mental exercise may be used to increase attention. The method of directing mental exercise may be used to increase relaxation. The method of directing mental exercise may be used to increase happiness. The method of directing mental exercise may be used to increase focus. The method of directing mental exercise may be used to increase learning. The method may further include varying a timing of the providing the next instruction based on the determined attribute. The method may further include determining a timing of providing the next instruction based on the determined attribute. The method may further include determining a frequency of providing the next instruction based on the determined attribute. The method may further include determining a probability of providing the next instruction based on the determined attribute. The method may further include receiving an input that indicates the user's breathing, and the determination of the next instruction may be based on the input that indicates the user's breathing. The received input that characterizes the user's internal felt sense may be an estimate made by the user. The estimate made by the user may be a qualitative estimate. The estimate made by the user may be a quantitative estimate. The determined attribute may be a position along a continuum. The method may further include providing, on a display screen of the computing device, a moving object, and the instruction for the user to perform the mental exercise may instruct the user to provide the input that characterizes the user's internal felt sense based in part of the moving object. The moving object may include a geometric shape. The geometric shape may be a circle. The moving object may move at a predetermined speed. The moving object may move at a variable speed based on a rate of user input. The method may further include determining a performance of the user, and the moving object may moves at a variable speed based on the performance of the user. The stimulus may be derived based on brain imaging information. The instruction may be derived based on brain imaging information. The mental exercise may be derived based on brain imaging information. The input that characterizes the user's internal felt sense may be received at the user interface as a selection of one or more buttons. The input that characterizes the user's internal felt sense may be received at the user interface as a position of one or more sliders. The input that characterizes the user's internal felt sense may be received at the user interface as one or more form input elements. The input that characterizes the user's internal felt sense may be received at the user interface as a cursor position. The input that characterizes the user's internal felt sense may be received at the user interface as a touch screen position. The input that characterizes the user's internal felt sense may be received at the user interface as a voice recognition. The input that characterizes the user's internal felt sense may be received at the user interface as one or more eye movements. The method may further include: (i) receiving, at a receiver of the computing device, an electronic message that includes an instruction to perform a mental exercise, (ii) testing the received instruction to perform a mental exercise, and (iii) providing, by the second output component of the computing device, the received instruction to preform the mental exercise. The directing mental exercise may be a game. The method may be used with psychological counseling. The score may be based on a change in a symptom of the user. The first stimulus and the next stimulus may include one or more sounds, and the next stimulus may include a change in volume of the one or more sounds relative to a volume of the first stimulus. The input that characterizes the user's internal felt sense may characterize an emotional response to user's internal felt sense. The brain imaging information may include one or more real-time fMRI signals. The method may further include providing an instruction regarding breathing of the user.

### Computing Devices, Software and Hardware

Computing devices and computer systems described in this document that may be used to implement the systems, techniques, machines, and/or apparatuses can operate as clients and/or servers, and can include one or more of a variety of appropriate computing devices, such as laptops, desktops, workstations, servers, blade servers, mainframes, mobile computing devices (e.g., PDAs, cellular telephones, smartphones, and/or other similar computing devices), tablet computing devices, computer storage devices (e.g., Universal Serial Bus (USB) flash drives, RFID storage devices, solid state hard drives, hard-disc storage devices), and/or other similar computing devices. For example, USB flash drives may store operating systems and other applications, and can include input/output components, such as wireless transmitters and/or USB connector that may be inserted into a USB port of another computing device.

Such computing devices may include one or more of the following components: processors, memory (e.g., random access memory (RAM) and/or other forms of volatile memory), storage devices (e.g., solid-state hard drive, hard disc drive, and/or other forms of non-volatile memory), high-speed interfaces connecting various components to each other (e.g., connecting one or more processors to memory and/or to high-speed expansion ports), and/or low speed interfaces connecting various components to each other (e.g., connecting one or more processors to a low speed bus and/or storage devices). Such components can be interconnected using various busses, and may be mounted across one or more motherboards that are communicatively connected to each other, or in other appropriate manners. In some implementations, computing devices can include pluralities of the components listed above, including a plurality of processors, a plurality of memories, a plurality of types of memories, a plurality of storage devices, and/or a plurality of buses. A plurality of computing devices can be connected to each other and can coordinate at least a portion of their computing resources to perform one or more operations, such as providing a multi-processor computer system, a computer server system, and/or a cloud-based computer system.

Processors can process instructions for execution within computing devices, including instructions stored in memory and/or on storage devices. Such processing of instructions can cause various operations to be performed, including causing visual, audible, and/or haptic information to be output by one or more input/output devices, such as a display that is configured to output graphical information, such as a graphical user interface (GUI). Processors can be implemented as a chipset of chips that include separate and/or multiple analog and digital processors. Processors may be implemented using any of a number of architectures, such as a CISC (Complex Instruction Set Computers) processor architecture, a RISC (Reduced Instruction Set Computer) processor architecture, and/or a MISC (Minimal Instruction Set Computer) processor architecture. Processors may provide, for example, coordination of other components computing devices, such as control of user interfaces, applications that are run by the devices, and wireless communication by the devices. Memory can store information within computing devices, including instructions to be executed by one or more processors. Memory can include a volatile memory unit or units, such as synchronous RAM (e.g., double data rate synchronous dynamic random access memory (DDR SDRAM), DDR2 SDRAM, DDR3 SDRAM, DDR4 SDRAM), asynchronous RAM (e.g., fast page mode dynamic RAM (FPM DRAM), extended data out DRAM (EDO DRAM)), graphics RAM (e.g., graphics DDR4 (GDDR4), GDDR5). In some implementations, memory can include a non-volatile memory unit or units (e.g., flash memory). Memory can also be another form of computer-readable medium, such as magnetic and/or optical disks.

Storage devices can be capable of providing mass storage for computing devices and can include a computer-readable medium, such as a floppy disk device, a hard disk device, an optical disk device, a Microdrive, or a tape device, a flash memory or other similar solid state memory device, or an array of devices, including devices in a storage area network or other configurations. Computer program products can be tangibly embodied in an information carrier, such as memory, storage devices, cache memory within a processor, and/or other appropriate computer-readable medium. Computer program products may also contain instructions that, when executed by one or more computing devices, perform one or more methods or techniques, such as those described above.

High speed controllers can manage bandwidth-intensive operations for computing devices, while the low speed controllers can manage lower bandwidth-intensive operations. Such allocation of functions is exemplary only. In some implementations, a high-speed controller is coupled to memory, display (e.g., through a graphics processor or accelerator), and to high-speed expansion ports, which may accept various expansion cards; and a low-speed controller is coupled to one or more storage devices and low-speed expansion ports, which may include various communication ports (e.g., USB, Bluetooth, Ethernet, wireless Ethernet) that may be coupled to one or more input/output devices, such as keyboards, pointing devices (e.g., mouse, touchpad, track ball), printers, scanners, copiers, digital cameras, microphones, displays, haptic devices, and/or networking devices such as switches and/or routers (e.g., through a network adapter).

Displays may include any of a variety of appropriate display devices, such as TFT (Thin-Film-Transistor Liquid Crystal Display) displays, OLED (Organic Light Emitting Diode) displays, touchscreen devices, presence sensing display devices, and/or other appropriate display technology. Displays can be coupled to appropriate circuitry for driving the displays to output graphical and other information to a user.

Expansion memory may also be provided and connected to computing devices through one or more expansion interfaces, which may include, for example, a SIMM (Single In Line Memory Module) card interfaces. Such expansion memory may provide extra storage space for computing devices and/or may store applications or other information that is accessible by computing devices. For example, expansion memory may include instructions to carry out and/or supplement the techniques described above, and/or may include secure information (e.g., expansion memory may include a security module and may be programmed with instructions that permit secure use on a computing device).

Computing devices may communicate wirelessly through one or more communication interfaces, which may include digital signal processing circuitry when appropriate. Communication interfaces may provide for communications under various modes or protocols, such as GSM voice calls, messaging protocols (e.g., SMS, EMS, or MMS messaging), CDMA, TDMA, PDC, WCDMA, CDMA2000, GPRS, 4G protocols (e.g., 4G LTE), and/or other appropriate protocols. Such communication may occur, for example, through one or more radio-frequency transceivers. In addition, short-range communication may occur, such as using a Bluetooth, Wi-Fi, or other such transceivers. In addition, a GPS (Global Positioning System) receiver module may provide additional navigation- and location-related wireless data to computing devices, which may be used as appropriate by applications running on computing devices.

Computing devices may also communicate audibly using one or more audio codecs, which may receive spoken information from a user and convert it to usable digital information. Such audio codecs may additionally generate audible sound for a user, such as through one or more speakers that are part of or connected to a computing device. Such sound may include sound from voice telephone calls, may include recorded sound (e.g., voice messages, music files, etc.), and may also include sound generated by applications operating on computing devices.

Computing devices can also include one or more sensors through which various states of and around the computing devices can be detected. For example, computing devices can include one or more accelerometers that can be used to detect motion of the computing devices and details regarding the detected motion (e.g., speed, direction, rotation); one or more gyroscopes that can be used to detect orientation of the computing devices in 3D space; light sensors that can be used to detect levels of ambient light at or around the computing devices; touch and presence sensors that can be used to detect contact and/or near-contact with one or more portions of the computing devices; environmental sensors (e.g., barometers, photometers, thermometers) that can detect information about the surrounding environment (e.g., ambient air temperature, air pressure, humidity); other motion sensors that can be used to measure acceleration and rotational forces (e.g., gravity sensors, rotational vector sensors); position sensors that can be used to detect the physical position of the computing devices (e.g., orientation sensors, magnetometers), and/or other appropriate sensors.

Various implementations of the systems, devices, and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications, or code) can include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor.

To provide for interaction with a user, the systems and techniques described here can be implemented on a computer having a display device (e.g., LCD display screen, LED display screen) for displaying information to users, a keyboard, and a pointing device (e.g., a mouse, a trackball, touchscreen) by which the user can provide input to the computer. Other kinds of devices can be used to provide for interaction with a user as well; for example, feedback provided to the user can be any form of sensory feedback (e.g., visual feedback, auditory feedback, and/or tactile feedback); and input from the user can be received in any form, including acoustic, speech, and/or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

The above description provides examples of some implementations. Other implementations that are not explicitly described above are also possible, such as implementations based on modifications and/or variations of the features described above. For example, the techniques described above may be implemented in different orders, with the inclusion of one or more additional steps, and/or with the exclusion of one or more of the identified steps. Additionally, the steps and techniques described above as being performed by some computing devices and/or systems may alternatively, or additionally, be performed by other computing devices and/or systems that are described above or other computing devices and/or systems that are not explicitly described. Similarly, the systems, devices, and apparatuses may include one or more additional features, may exclude one or more of the identified features, and/or include the identified features combined in a different way than presented above. Features that are described as singular may be implemented as a plurality of such features. Likewise, features that are described as a plurality may be implemented as singular instances of such features. The drawings are intended to be illustrative and may not precisely depict some implementations. Variations in sizing, placement, shapes, angles, and/or the positioning of features relative to each other are possible.

TABLE 1

| MEDICATION NAME | CATEGORY AND USES |
|---|---|
| Acamprosate | Decreases the desire to drink alcohol. |
| acetaminophen | Treats minor aches and pain, reduces |
| | fever. |
| Acetaminophen/Aspirin/ | Relieves headaches, migranes, aches. |
| Caffeine | |
| Acetaminophen/Butalbital/ | Treats tension headaches. |
| Caffeine | |
| Acetaminophen/Butalbital/ | Relieve pain. |
| Caffeine/Codeine | |
| Acetaminophen/Codeine | Mild to moderately severe pain. |
| Acetaminophen/Hydrocodone | Moderate to moderately severe pain. |
| Acetaminophen/Oxycodone | Moderate to moderately severe pain. |
| | Narcotic pain reliever. |
| Acetaminophen/ | Mild to moderate pain. Naroctic pain |
| Pentazocine | medicine. |
| Acetaminophen/ | Minor pain, reduces fever, relieves cold |
| Phenyltoloxamine | and flu symptoms. |
| Acetaminophen/ | Mild to moderate pain. Naroctic pain |
| Propoxyphene | medicine. |
| Acetaminophen/ | Treating short term (5 days or less) pain. |
| Tramadol | Analygesic combination. |
| Acetazolamide | Glaucoma, types of epilepsy, mountain |
| | sickness. Group of drugs called carbonic |
| | anhydrase inhibitors. |
| Alfentanil | Used during surgery to control pain and |
| | keep you asleep. |
| almotriptan | Migrane headaches. |
| Alprazolam | Anxiety and panic disorder. |
| Alteplase | Treating blood clots in the lungs. |
| | Improve heart function. Improve |
| | recovery in stroke patients. |
| Atracurium | Neuromuscular blocking drug or skeletal |
| | muscle relaxant, used during surgery. |
| Aspirin | Arthritis, headache, toothache, menstrual |
| | pain. |
| Amantadine | Treats/prevents the flu. Parkinson's |
| | disease & parkinson-like symptoms. |
| Ambenonium | Treating myasthenia gravis. |
| Amitriptyline | Depression treatment - a TCA. |
| Amobarbital | Sedative-hypnotic properties. |
| | Barbiturate derivative. White crystalline |
| | powder with no odor and a bitter taste. |
| Amoxapine | Depression treatment - tricyclic |
| | antidepressant. |
| Amphetamine | CNS stimulant to treat attention deficit |
| | hyperactivity disorder & narcolepsy. |
| amphetamine/ | ADHD and narcolepsy. Stimulant. |
| dextroamphetamine | |
| Apomorphine | Parkinson's disease - treats loss of |
| | control of muscle movements. |
| Aprepitant | Prevents nausea and vomiting caused by |
| | chemotherapy or surgery. |
| Aripiprazole | Schizophrenia, bipolar disorder, and |
| | depression treatment. Also treats |
| | irritability associated with autism. |
| Armodafinil | Treats sleepiness from narcolepsy, sleep |
| | apnea, night shift work. |
| Asenapine | Schizophrenia and bipolar disorder |
| | treatment. |
| Aspirin | Pain, fever, arthritis, inflammation |
| | treatment. Reduce risk of heart attack. |
| Aspirin/Butalbital/ | Tension headaches and mild to |
| Caffeine | moderate pain. Butalbital is a |
| | barbiturate. |
| Aspirin/Butalbital/ | Pain. |
| Caffeine/Codeine | |
| Aspirin/Caffeine/ | Moderate to severe pain. Analgesic |
| Dihydrocodone | combination. |
| Aspirin/Caffeine/ | Treats pain and relaxes muscles. |
| Orphenadrine | Muscle relaxant. |
| Aspirin/Caffeine/ | Mild to moderate pain. Narcotic |
| Propoxyphene | analgesic, caffeine, and salicylate. |
| Aspirin/Carisoprodol | Muscle relaxant. |
| Aspirin/Carisoprodol/ | Opioid pain medication. |
| Codeine | |
| Aspirin/Hydrocodone | Moderate to severe pain. |
| Aspirin/Meprobamate | Pain, anxiety, and tension. |
| Atomoxetine | treats ADHD. |
| Azathioprine | Prevents body from rejecting kidney |
| | transplant. Treats joint pain & swelling |
| | from rheumatoid arthritis. |
| Baclofen | Treats muscle spasms caused by |
| | multiple sclerosis, cerebral palsy, or |
| | damage to brain/spinal cord. Muscle |
| | relaxer. |
| Belladonna/ | Typically used as a suppository to treat |
| Opium | moderate to severe pain due to spasms |
| | of urinary tract. |
| Benzocaine/ | Nausea/vomiting. |
| Trimethobenzamide | |
| Benzotropine | Treats symptoms of Parkinson disease. |
| Benzphetamine | Used as a short term adjunct in |
| | management of exogenous obesity. |
| Biperiden | Parkinson's disease or side effects of |
| | other drugs. |
| Botulinum | Reducint severity of abnormal head |
| Toxin Type B | position and neck pain associated with |
| | certain neck problems. Blocks nerve |
| | impulses to muscles, temporarily |
| | paralyzing muscle. |
| Bromocriptine | Treats menstrual problems, growth |
| | hormone overproduction, Parkinson |
| | disease, and pituitary tumors. Stops |
| | breast milk production. Helps control |
| | blood sugar levels in patients with type 2 |
| | diabetes. |
| Bromocriptine | Treats type 2 diabetes. Dopamine |
| Mesylate | receptor agonist. |
| Buprenorphine | Treatment of opioid dependence, |
| Hydrochloride | moderate to severe pain, management of |
| | moderate to severe chronic pain. |
| buprenorphine | Moderate to severe chronic pain. Opioid |
| | addiction and dependence. |
| Buprenorphine/ | Treats opoid dependence, addiction or |
| Naloxone | dependence to narcotic medicine. |
| Bupropion | Depression. Aids in quitting smoking. |
| | Prevents depression caused by |
| | Seasonal Affective Disorder. |
| | Antidepressant. |
| Bupropion/ | Obesity treatment. |
| Naltrexone | |
| Buspirone | Treats anxiety. |
| Butabarbital | Treats insomnia. Used before |
| | surgery/procedure. Barbiturate. |
| Butalbital | Barbiturate often combined with other |
| | medications. Treats pain/headache. |
| butorphanol | Treats pain. |
| Butorphanol | Moderate to severe pain, muscle pain, |
| Tartrate Aerosol | migrane headaches. |
| Cabergoline | Lowers high levels of prolactin in your |
| | blood. Hyperprolactinaemia, Parkinson's |
| | disease. |
| Caffeine | CNS stimulant. Most widely consumed |
| | psychoactive drug. |
| caffeine/ | Treats/prevents migraine and cluster |
| ergotamine | headaches. |
| Carbamazepine | Anti-epileptic agent, mood stabilizer, |
| | carboxamide. |
| Carbamazepine | Anti-epileptic agent, mood stabilizer, |
| | carboxamide. |
| Carbidopa | Parkinson's disease treatment (shaking, |
| | stiffness, slow movement) |
| Carbidopa/Levodopa | Parkinson's disease treatment (shaking, |
| | stiffness, slow movement) |
| Carbidopa/Levodopa/ | Parkinson's disease treatment. |
| Entacapone | |
| Carisoprodol | Muscle relaxant. Treats pain and |
| | stiffness of muscle spasms. |
| Celecoxib | Anti-inflammatory. Treats pain, arthritis. |
| | Non-steroidal. |
| Cevimeline | Treats dry mouth from Sjogren |
| | syndrome. Cholinergic Agonist. |
| Chloral Hydrate | Treats insomnia. Used before |
| | surgery/procedure. Barbiturate. |
| Chlordiazepoxide | Treats anxiety, symptoms of alcohol |
| | withdrawal, and tremor. |
| | Benzodiazepine. |
| Chlorphenesin | Muscle relaxant. |
| Chlorpromazine | Treats mental disorders, severe behavior |
| | disorders, sever hiccups, nausea and |
| | vomiting, and types of porphyria. Used |
| | before and after surgery to relieve |
| | anxiety. Phenothiazine. |
| Chlorzoxazone | Muscle relaxant. Treats pain and |
| | stiffness of muscle spasms. |
| cholesterol | Organic molecule. |
| Choline | Water soluble essential nutrient grouped |
| | with B vitamins. |
| Choline | Fever, inflammation. |
| Salicylate/Magnesium | |
| Salicylate | |
| Cisatracurium | Relaxes muscles during surgery. |
| Citalopram | SSRI that treats depression. |
| Citalopram | Antidepressant. |
| Hydrobromide | |
| Clobazam | Benzodiazepine that treats seiizures |
| | from Lennox-Gastaut syndrome. |
| Clomipramine | Antidepressant. Tricycline that treats |
| | OCD, panic discorder, depressive |
| | disorder. |
| Clonazepam | Benzodiazepine that treats seizures, |
| | panic disorder and anxiety. |
| Clonidine | High blood pressure. ADHD. |
| | Antihypertensives. |
| Clopidogrel | Blood thinner to prevent stroke, heart |
| | attack and other heart problems. |
| Clorazepate | Benzodiazepine. Treats anxiety, trouble |
| | sleeping, symptons of alcohol |
| | withdrawal, certain types of epilepsy. |
| Clozapine | Atypical antipsychotic. Treats |
| | schizophrenia. Lowers the risk of |
| | suicidal behavior in patients with |
| | schizophrenia. |
| Codeine | Analgesic, Opiate, Antidiarrhoeal. Treats |
| | pain or cough. |
| Codeine Phosphate/ | Pain. |
| Acetaminophen | |
| Codeine Phsophate/Aspirin/ | Pain. |
| Caffeine/Butalbital | |
| Codeine Sulfata | Mild to moderate pain. |
| Cyclizine | Antihistamine to treat nausea, vomiting, |
| | and dizziness. Motion sickness, vertigo. |
| Cyclobenzaprine | Muscle relaxant. Treat skeletal muscle |
| | conditions such as pain or injury. |
| Dalfampridine | Helps improve walking in patients with |
| | multiple sclerosis. |
| Dantrolene | Muscle relaxant. Muscle spasms caused |
| | by MS, cerebral palsy, damage to |
| | brain/spinal cord. Treat and prevent |
| | symptoms of malignant hyperthermia. |
| Darifenacin | Treats symptoms of overactive bladder |
| | (incontinence, frequency) |
| Desflurane | General anaesthetic. Type of |
| | anesthesia. |
| Desipramine | Antidepressant. ADHD, substance- |
| | related disorders, depression. |
| Desvenlafaxine | SNRI. Treats depression. Major |
| | depression. |
| Dexamethasone | Corticosteroid. Treats inflammation and |
| | many other medical problems. |
| Dexmedetomidine | Keeps you asleep during surgery. |
| Dexmethylphenidate | CNS stimulant. Treats ADHD. |
| Dextroamphetamine | CNS stimulant. Treats ADHD. |
| Dextromethorphan | Treats cough caused by colds, flu. |
| Dextromethorphan/ | Antiarrhythmic. Treats emotional |
| Quinidine | incontinence or uncontrollable crying or |
| | laughing. |
| Diazepam | Benzodiazepine, hypnotic. Treats |
| | anxiety, muscle spasms, seizures. |
| Diclofenac | Anti-inflammatory. Treats actinic |
| | keratoses. Pain and swelling by arthritis. |
| Diclofenac/ | Anti-inflammatory. Treats arthrisis pain. |
| Misoprostal | |
| Diethylpropion | Used for short periods as part of a diet |
| | plan to lose weight. Amine Anorectic. |
| Diflunisal | Pain, rheuatoid arthritis, osteoarthritis. |
| Dihydrocodeine | Analgesic. Pain or severe dyspnea. |
| Dihydroergotamine | Migrane, cluster headaches, status |
| | migrainosus. |
| Dihydroergotamine | Migrane headaches. |
| Mesylate | |
| Dihydroergotamine | Migraine headaches. |
| systemic | |
| Dimenhydrinate | Prevent motion sickness. |
| Diphenhydramine | Antiemetic, histamine. Hives, common |
| | cold, nausea, motion sickness. |
| Diphenhydramine/ | Antiemetic, histamine. Hives, common |
| Naproxen | cold, nausea, motion sickness. |
| Disulfiram | Alcoholism, alcohol abuse, addictive |
| | personality treatment. |
| Divalproex | Mood stabilizer, anti-epileptic. Treats |
| | seizures, manic phase of bipolar, |
| | migrane headaches. |
| Doclofenac | Anti-inflammitory. Treats actinic |
| | keratoses. Pain and swelling from |
| | arthritis. |
| Dolasetron | Prevents and treats nausea and vomiting |
| | after surgery. |
| Donzepezil | Treats symptoms of Alzheimer's disease. |
| Donzepezil/ | Treats symptoms of Alzheimer's disease. |
| Memantine | |
| Doxacurium | Muscle relaxant used in anesthesia. |
| Doxapram | Respitory stimulant. |
| Doxepin | Antidepressant. Depression, anxiety, |
| | sleep disorders. |
| Doxylamine | Anticholinergic. Treats insomnia. Treats |
| | hay fever, allergies. |
| Doxylamine/ | Management of nausea/vomiting of |
| Pyridoxine | pregnancy or morning sickness. |
| Dronabinol | Nausea and vomiting caused by cancer |
| | medications. |
| Droperidol | Antiemetic. Treats anxiety, nausea, |
| | vomiting before/after surgery. |
| | Psychosis. |
| Duloxetine | SSRI. Treats depression, anxiety, |
| | diabetic peripheral neuropathy, |
| | fibromyalgia, chronic muscle/bone pain. |
| eletriptan | Treats migrane headaches. |
| Entacapone | Parkinson's disease. |
| Ergoloid Mesylates | Confusion, diziness, depressed mood. |
| ergotamine | Constricts blood vessels. |
| Escitalopram | SSRI that treats depression and |
| | generalized anxiety disorder. |
| Esomeprazole/ | anti-inflammatory |
| Naproxen | |
| Estazolam | Benzodiazepine that treats insomnia. |
| Eszopiclone | Treats insomnia. |
| Ethchlorvynol | Sedative and hypnotic medication. |
| Ethosuximide | Anti-epileptic agent - treats seizures. |
| Ethotoin | Anti-epileptic agent |
| Etodolac | Anti-inflammatory - pain from arthritis |
| | and other medical problems. |
| Etomidate | Anaesthesia. |
| Ezogabine | Anti-convulsant. |
| Famotidine/ | Arthritis. |
| Ibuprofen | |
| Felbamate | Anti-epileptic agent. |
| Fenoprofen | Anti-inflammatory - treats pain. |
| fentanyl | Analgesic, opoid. Treats moderate to |
| | severe pain. Narcotic. |
| Fesoterodine | Overactive bladder. |
| Fingolimod | Reduces flare-ups in those with MS. |
| Flumazenil | Benzodiazepine. Treats drowsiness |
| | caused by sedative medicines. |
| Fluoxetine | SSRI. Depression, OCD. |
| Fluphenazine | Schizophrenia & different types of |
| | behavior problems. Phenothiazine. |
| Flurazepam | Benzodiazepine. Treats insomnia. |
| Flurbiprofen | Anti-inflammatory. Keeps pupils of the |
| | eyes from getting smaller during eye |
| | surgery. |
| Fluvoxamine | SSRI. OCD treatment. Depression, |
| | panic disorder. |
| Fosaprepitant | Antiemetic. Prevents nausea and |
| | vomiting that is caused by |
| | chemotherapy. |
| Fosphenytoin | Anti-epileptic. Anticonvulsant. |
| Fospropofol | Relax or sleep during/after surgery. |
| | Strong sedative. |
| frovatriptan | Treats migranes. |
| Gabapentin | Threats seizures. Treats Restless Leg |
| | Syndrome. |
| gabapentin | Threats seizures. Treats Restless Leg |
| enacarbil | Syndrome. |
| Galantamine | Treats dimentia. Alzheimer's disease, |
| | Vascular dementia. |
| Gamma | CNS depressant. Treats loss of muscle |
| Hydroxybutyric | control and excessive daytime |
| Acid | sleepiness caused by narcolepsy |
| Glatiramer Acetate | Immunomodulator drug used to treat MS. |
| Granisetron | Antiemetic. Prevents nausea and |
| | vomiting that is caused by |
| | chemotherapy. |
| Guanfacine | Antihypertensive. Treats high blood |
| | pressure. ADHD. |
| Halazepam | benzodiazepine. |
| Haloperidol | Antipsychotic. Schizophrenia, behavior |
| | problems, agitation, Tourette's. |
| hydrocodone | Semi-synthetic opioid. Pain |
| Hydrocodone | Pain. |
| Bit/Acetaminophen | |
| Hydrocodone/ | Pain. |
| Acetaminophen | |
| Hydrocodone/ | Pain. |
| Ibuprofen | |
| hydromorphone | Opoid, Analgesic. Moderate to severe |
| | chronic pain. |
| Hydromorphone HCl | Opoid, Analgesic. Moderate to severe |
| | chronic pain. |
| Hydroxyzine | Analgesic. Anxiety, tension, |
| | nervousness, nausea, vomiting, |
| | allergies, skin rash, hives, itching. |
| | Antihistamine. |
| Ibuprofen | Analgesic, Anti-anflammatory. Pain and |
| | fever. |
| Ibuprofen/ | Analgesic, Opioid, anti-inflammatory. |
| Hydrocodone | Pain and inflamation. |
| Iloperidone | Antipsychotic. Schizophrenia. |
| Imipramine | Antidepressant. Depression. Treats |
| | bedwetting in children. |
| IncobotulinumtoxinA | Muscle freeze |
| Indomethacin | Pain, inflammation, arthritis, |
| | osteoarthritis. Gout, bursitis, tendonitis. |
| Interferon Beta-1a | Multiple sclerosis, melanoma, multiple |
| | myeloma. |
| Isoflurane | General anesthesia. |
| Isometheptene | Migranes and tension headaches. |
| Ketamine | Anaesthetic. |
| Ketoprofen | Anti-inflammatory, analgesic. Pain. |
| Ketorolac | Pain and inflammation. Arthritis, cramps, |
| | medical problems. |
| Lacosamide | Anti-epileptic. Anticonvulsant. |
| Lamotrigine | Mood stabilizer, anti-epileptic. Treats |
| | seizures, manic phase of bipolar, |
| | migrane headaches. |
| Lansoprazole/ | Arthritis. |
| Naproxen | |
| Levetiracetam | Anti-epileptic. Treats seizures. |
| Levodopa | Parkinson's disease. |
| Levomethadyl | Treatment to opioid dependence. |
| Acetate | |
| Levorphanol | Opioid analgesic. |
| Tartrate | |
| Lisdexamfetamine | CNS stimulant. ADHD. |
| Lithium | Mood stabilizer. Treats mania in bi- |
| | polar. |
| Lorazepam | Benzodiazepine. Treats anxiety, anxiety |
| | with depression, insomnia. |
| Lorcaserin | Weightless drug |
| Loxapine | Antipsychotic. Schizophrenia and mental |
| | disorder. |
| Lurasidone | Antipsychotic. Schizophrenia and mental |
| | disorder. |
| Magnesium Sulfate | Preeclampsia during pregnancy. |
| Maprotiline | Antidepressant. Depression and anxiety. |
| | Panic, panic disorder. |
| Mazindol | Stimulant. Anorectic. |
| Meclizine | Antiemetic. Prevents and controls |
| | nausea, vomiting, dizziness, vertigo. |
| Mefenamic acid | anti-inflammatory. Pain, menstrual pain. |
| Melatonin | Anticipates onset of darkness. |
| Meloxicam | Anti-inflammatory. Osteoarthritis and |
| | rheumatoid arthritis. |
| Memantine | Treats dementia. |
| meperidine | Pain. |
| meperidine | Pain. |
| Meperidine/ | Pain relief. |
| Promethazine | |
| Mephenytoin | Hydantoin, anticonvulsant. |
| Mephobarbital | Anti-epileptic. |
| Meprobamate | Tension, anxiety, nervousness. Muscle |
| | spasm, headache. Tranquilizer. |
| Metaxalone | Pain, muscle spasm, spasiticity, cramps |
| methadone | Opioid, analgesic. Moderate to severe |
| | pain, treatment of narcotic drug |
| | addiction. |
| methadone | Opioid, analgesic. Moderate to severe |
| | pain, treatment of narcotic drug |
| | addiction. |
| Methamphetamine | CNS Stimulant. ADHD, weight loss in |
| | obese. |
| Methocarbamol | Muscle relaxant. Muscle pain and |
| | spasms. |
| Methohexital | Barbituarate. Fall asleep during surgery. |
| | Anesthetic. |
| Methsuximide | Anti-epileptic. Ansence seizure. |
| methylphenidate HCl | CNS Stimulant. Mild. ADHD. |
| methysergide | Migrane headaches. |
| Metoclopramide | Antiemetic. Treats gastric esophageal |
| | reflux disease. Nausea, vomiting, and |
| | heartburn caused by stomach problems. |
| Miglustat | Treats type 1 Gaucher disease. |
| Milnacipran | Fibromyalgia. SNRI. |
| Mirabegron | Overactive bladder. |
| Mirtazapine | Antidepressant. Depression, depression |
| | disorder and major depression |
| Misoprostol | Prevents stomach ulcers caused by anti- |
| | inflammatory drugs |
| Mivacurium | Neuromuscular blocking drug or skeletal |
| | muscle relaxant, used during surgery. |
| Modafinil | Excessive uncontrollable daytime |
| | sleepiness, ADHD, fatigue, obstructive |
| | sleep apnea. |
| Molindone | Antipsychotic. Schizophrenia. |
| Morphine | Analgesic, opioid. Moderate to severe |
| | pain. |
| Morphine Liposomal | Analgesic, opioid. Moderate to severe |
| | pain. |
| Morphine Sulfate | Analgesic, opioid. Moderate to severe |
| | pain. |
| Morphine/ | Analgesic, opioid. Moderate to severe |
| Naltrexone | pain. |
| Nabilone | Cannabinoid. Treats and prevents |
| | nausea and vomiting caused by cancer |
| | medicines. |
| Nabumetone | Anti-inflammatory. Pain caused by |
| | arithritis. |
| nalbuphine | Opioid Agonist. Treats various types of |
| | severe pain. |
| Naloxone | Septic shock, respitatiory disorders, |
| | referse effects of certain medicines. |
| Naloxone/ | Opioid Analgesic. |
| Oxycodone | |
| Naltrexone HCl | Pure opioid antagonist. Blocks the |
| | subjective effects of intravenously |
| | administered opioids. |
| Naproxen | Fever and pain. Arthritis, gout, |
| | menstrual cramps, tendinitis. |
| naproxen/ | Acute migrane attacks. |
| sumatriptan | |
| naratriptan | Migraine headaches. |
| Natalizumab | Multiple sclerosis. Crohn's disease and |
| | MS. |
| Nefazodone | Depression. PTSD. |
| Neostigmine | Myasthenia gravis. Reverses effects of |
| | anesthesia. |
| Netupitant/ | Nausea and vomiting. |
| Palonosetron | |
| Nicotine | Helps to quit smoking. Ulcerative colitis. |
| | Tobacco abuse. |
| Nimodipine | Reduce brain damage caused by |
| | bleeding in the brain. |
| Nortriptyline | Antidepressant. ADHD, anxiety disorder, |
| | enuresis. |
| Olanzapine | Antipsychotic. Psychotic mental |
| | disorders, schizophrenia or bipolar |
| | disorder. |
| OnabotulinumtoxinA | Stops muscle activity |
| Ondansetron | Antiemetic. SSRI. Prevents nausea and |
| | vomiting. |
| opium | Analgesic. |
| Orphenadrine | Muscle relaxant. Pain, muscle spasms, |
| | cramps, muscle rigidity. |
| Oxaprozin | Treats pain from arthritis. Osteoarthritis, |
| | chronic childhood arthritis. |
| Oxazepam | Anxiety, anxiety with depression. |
| | Alcohol withdrawal, partial seizure. |
| Oxcarbazepine | Anti-epileptic. Treats seizures. |
| Oxybutynin | Overactive bladder. |
| oxycodone | Moderate to severe pain when around |
| | the clock pain relief is needed. Narcotic. |
| | Opioid and analgesic. |
| Oxycodone HCl/ | Pain relief. |
| Acetaminophen | |
| Oxycodone | Moderate to severe pain. |
| HCl/Ibuprofen | |
| Oxycodone/Aspirin | Treats pain. |
| oxymorphone | Moderate to severe pain. |
| Paliperidone | Antipsychotic. Schizophrenia. |
| Palonosetron | Nausea and vomiting caused by cancer |
| | treatments. |
| Pancuronium | Muscle relaxer. |
| Paramethadione | Anticonvulsant. |
| Paroxetine | Major depression, OCD, PMDD, GAD, |
| | PTSD |
| Pemoline | ADHD, Daytime sleepiness |
| Pentazocine | Moderate to severe pain. |
| Pentazocine HCl/ | Moderate to severe pain. |
| Naloxone HCl | |
| Pentobarbital | Relieves tension, anxiety, nervousness, |
| | insomnia. |
| Perampanel | Anti-epileptic |
| Perphenazine | Schozophrenia, psychosis, vomiting, |
| | nausea. |
| Phendimetrazine | Weightless. |
| Phenelzine | Antidepressant and anxiolytic. |
| Phenobarbital | Treats epilepsy. |
| Phentermine | Weight loss plan. |
| Phentermine/ | Weight loss. |
| Topiramate | |
| Phenytoion | Anti-epileptic. Treats seizures, |
| | anticonvulsant. |
| Phosphorated | Nausea and vomiting. |
| Carbohydrate | |
| Solution | |
| Pilocarpine | Dry mouth caused by radiation treatment |
| | or Sjogren syndrome. |
| Pimozide | Antipsychotic. Tourette syndrome, |
| | psychosis, Huntington's disease. |
| Piroxicam | Treats pain, inflammation, arthritis. |
| Pramipexole | Treats Parkinson's disease, restless leg |
| | syndrome, depressive disorder. |
| Prednisone | Inflammation, severe allergies, |
| | complications of chronic |
| | illnesses. Steroid. |
| Pregabalin | Nerve and muscle pain caused by |
| | diabetes, shingles, fibromyalgia, spinal |
| | cord injury. |
| Primidone | Epilepsy, tremor, seizure disorder. |
| Prochlorperazine | Nausea and |
| | vomiting. Schizophrenia. Anxiety |
| | disorder, dementia, status migrainosus. |
| Procyclidine | Anticholinergic drug treating |
| | parkinsonism, akathisia and acute |
| | dystonia. |
| Promethazine | Motion sickness, nausea, vomiting, |
| | dizziness. Allergic reactions, helps |
| | people go to sleep. |
| Propofol | General anaesthetic. Relax or sleep |
| | before/after surgery. |
| propoxyphene | Mild narcotic analgesic. |
| Protriptyline | Antidepressant. |
| Pyridostigmine | Myasthenia gravis |
| Quazepam | Insomnia, sleep induction, sleep |
| | maintenance. |
| Quetiapine | Antipsychotic. Schizophrenia, bipolar |
| | disorder, depression. |
| Quinidine | Treats irregular heartbeat. Also treats |
| | malaria. |
| Ramelteon | Treats insomnia. |
| Rasagiline | Treats signs and symptoms of |
| | Parkinson's. |
| Remifentanil | Relieves pain during/after |
| | surgery. Opioid. |
| Riluzole | Treatment of amyotrophic lateral |
| | sclerosis. Treatment for Lou gehrig's |
| | disease. |
| Risperidone | Schizophrenia, bipolar disorder, |
| | dementia, tourettes. |
| Rivastigmine | Dementia with Alzheimer's disease, or |
| | Parkinson's disease. |
| rizatriptan | Migraine headaches. |
| Rocuronium | Relaxes muscles during surgery or |
| | medical procedures. |
| Rofecoxib | Anti-inflammatory drug. |
| Ropinirole | Parkinson's disease. Restless leg |
| | syndrome. |
| Rotigotine | Parkinson's disease. Restless leg |
| | syndrome. |
| Rufinamide | Seizures in patients with Lennox-Gastaut |
| | syndrome. |
| Salsalate | Anti-inflammatory drug. |
| Scopolamine | Nausea and vomiting. Motion |
| | sickness. Anesthesia and surgery. |
| Secobarbital | Treats insomnia and also makes you feel |
| | sleepy. |
| Selegiline HCl | Treats depression. |
| Sertraline | SSRI that treats depression, anxiety, |
| | major depression, OCD. |
| Sevoflurane | General anaesthetic. Causes you to |
| | become unconscious before surgery. |
| Sodium Oxybate | Treats loss of muscle control (cataplexy) |
| | and excessive daytime sleepiness |
| | caused by narcolepsy. |
| Solifenacin | Overactive bladder. |
| Succinylcholine | Relaxes muscles during surgery or other |
| | medical procedures. |
| Sufentanil | Treats pain. Medicine used along |
| | anesthetic medicine during surgery or |
| | during childbirth. |
| Sulindac | Treats pain caused by arthritis, gout, or |
| | sore tendons. |
| Sumatriptan | Migraine headaches. |
| Suvorexant | Insomnia. |
| Tacrine | Alzheimer's disease. |
| tapentadol | Treats moderate to severe pain. Treats |
| | nerve pain caused by diabetes. Narcotic |
| | pain reliever. |
| Tasimelteon | Sleep-wake disorder. |
| Temazepam | Insomnia. Benzodiazepine. |
| Teriflunomide | Helps with MS. |
| Tetrabenazine | Treats chorea caused by Huntington. |
| Thiethylperazine | Antiemetic. |
| Thiopental | Barbiturate. General anesthetic. |
| Thioridazine | Schizophrenia, Psychosis |
| Thiothixene | Schizophrenia, Psychosis |
| Tiagabine | Antiepilepsy. |
| Hydrochloride | |
| Tizanidine | Muscle spasms. Muscle relaxer. Lower |
| | back pain, cramps. |
| Tolcapone | Parkinson's disease. |
| Tolmetin | Treats pain and inflammation caused by |
| | arthritis. Osteoarthritis. |
| Tolterodine | Overactive bladder. |
| Topiramate | Treats and prevents seizures and |
| | prevents migrane |
| | headaches. Pain. Bipolar disorder, |
| | epileptic, infantile spasms. |
| Tramadol HCl | Moderate to severe pain. |
| Tranylcypromine | Depression. Posttraumatic stress |
| | disorder, depressive disorder. |
| Trazodone | Depression, sleep initiation, depressive |
| | disorder. |
| Triazolam | Insomnia, sleep initiation, maintenance |
| | disorders. |
| Trifluoperazine | Treats psychotic disorder and |
| | anxiety. Nausea and vomiting caused by |
| | chemotherapy. |
| Trihexyphenidyl | Antiparkinson. |
| Trimethadione | Oxazolidinedione |
| | anticonvulsant. Epileptic conditions. |
| Trimethobenzamide | Antiemetic. Nausea and vomiting. |
| Trimipramine | Antidepressant. Depression. |
| Trospium | Overactive bladder. |
| Tryptophan | Animo acid necessary and combined |
| | with other drugs. |
| Valdecoxib | anti-inflammatory. Osteoarthritis and |
| | rheumatoid arthritis. |
| Valporic Acid | Bipolar disorder, seizures, mood |
| | disorders, migraine headaches. |
| Valporic Acid | Bipolar disorder, seizures, mood |
| | disorders, migraine headaches. |
| Varenicline | Helps to quit smoking. |
| Vecuronium | Relaxes muscles. Surgery and other |
| | medical procedures. |
| Venlafaxine | Antidepressant, SSRI. Depression, |
| | generalized anxiety disorder, panic |
| | disorder, social anxiety disorder. |
| Vigabatrin | Anti-epileptic. Seizures and infantile |
| | spasms. |
| Vilazodone | Antidepressant. Major depressive |
| | disorder. |
| Zaleplon | Insomnia. Sleep initiation and |
| | maintenance disorders. |
| Ziconotide | Relieves severe chronic pain. |
| Ziprasidone | Antipsychotic. Treats schizophrenia, |
| | bipolar disorder. Tourette's syndrome. |
| Zolmitriptan | Treats migraine headaches. Triptan. |
| Zolpidem | Treats insomnia. |
| Zolpidem Tartrate | Sedative-hypnotic for short term pain. |
| Zonisamide | Anti-epileptic. Treats partial seizures in |
| | adults. |
| Anorexia Nervosa | Eating Disorders |
| Antisocial Personality | Personality Disorders |
| Disorder | |
| Anxiety Disorder Due | Anxiety Disorders |
| to Medical | |
| Condition | |
| Anxiety Disorder, | Anxiety Disorders |
| NOS | |
| Avoidant Personality | Personality Disorders |
| Disorder | |
| Bipolar Disorder NOS | Mood Disorders |
| Bipolar | Mood Disorders |
| I Disorder, Most | |
| Recent | |

TABLE 2

| Disorder | Category |
|---|---|
| Acute Stress Disorder | Anxiety Disorders |
| Adjustment Disorder | Adjustment Disorders |
| Unspecified | |
| Adjustment Disorder | Adjustment Disorders |
| with Anxiety | |
| Adjustment Disorder | Adjustment Disorders |
| with Depressed | |
| Mood | |
| Adjustment Disorder | Adjustment Disorders |
| with | |
| Disturbance of | |
| Conduct | |
| Adjustment Disorder | Adjustment Disorders |
| with Mixed | |
| Anxiety and | |
| Depressed Mood | |
| Adjustment Disorder | Adjustment Disorders |
| with Mixed | |
| Disturbance of | |
| Emotions and | |
| Conduct | |
| Agoraphobia without | Anxiety Disorders |
| History of | |
| Panic Disorder | |
| Episode Depressed, | |
| In Full Remission | |
| Bipolar | Mood Disorders |
| I Disorder, Most | |
| Recent | |
| Episode Depressed, | |
| In Partial Remission | |
| Bipolar | Mood Disorders |
| I Disorder, Most | |
| Recent | |
| Episode Depressed, | |
| Mild | |
| Bipolar | Mood Disorders |
| I Disorder, Most | |
| Recent | |
| Episode Depressed, | |
| Moderate | |
| Bipolar | Mood Disorders |
| I Disorder, Most | |
| Recent | |
| Episode Depressed, | |
| Severe With | |
| Psychotic Features | |
| Bipolar | Mood Disorders |
| I Disorder, Most | |
| Recent | |
| Episode Depressed, | |
| Severe Without | |
| Psychotic Features | |
| Bipolar | Mood Disorders |
| I Disorder, Most | |
| Recent | |
| Episode Depressed, | |
| Unspecified | |
| Bipolar | Mood Disorders |
| I Disorder, Most | |
| Recent | |
| Episode Manic, In Full | |
| Remission | |
| Bipolar | Mood Disorders |
| I Disorder, Most | |
| Recent | |
| Episode Manic, In | |
| Partial Remission | |
| Bipolar | Mood Disorders |
| I Disorder, Most | |
| Recent | |
| Episode Manic, Mild | |
| Bipolar | Mood Disorders |
| I Disorder, Most | |
| Recent | |
| Episode Manic, | |
| Moderate | |
| Bipolar | Mood Disorders |
| I Disorder, Most | |
| Recent | |
| Episode Manic, | |
| Severe With | |
| Psychotic Features | |
| Bipolar | Mood Disorders |
| I Disorder, Most | |
| Recent | |
| Episode Manic, | |
| Severe Without | |
| Psychotic Features | |
| Bipolar | Mood Disorders |
| I Disorder, Most | |
| Recent | |
| Episode Manic, | |
| Unspecified | |
| Bipolar | Mood Disorders |
| I Disorder, Most | |
| Recent | |
| Episode Mixed, In Full | |
| Remission | |
| Bipolar | Mood Disorders |
| I Disorder, Most | |
| Recent | |
| Episode Mixed, In | |
| Partial Remission | |
| Bipolar | Mood Disorders |
| I Disorder, Most | |
| Recent | |
| Episode Mixed, Mild | |
| Bipolar | Mood Disorders |
| I Disorder, Most | |
| Recent | |
| Episode Mixed, | |
| Moderate | |
| Bipolar | Mood Disorders |
| I Disorder, Most | |
| Recent | |
| Episode Mixed, | |
| Severe With | |
| Psychotic Features | |
| Bipolar | Mood Disorders |
| I Disorder, Most | |
| Recent | |
| Episode Mixed, | |
| Severe Without | |
| Psychotic Features | |
| Bipolar | Mood Disorders |
| I Disorder, Most | |
| Recent | |
| Episode Mixed, | |
| Unspecified | |
| Bipolar | Mood Disorders |
| I Disorder, Most | |
| Recent | |
| Episode Unspecified | |
| Bipolar I Disorder, | Mood Disorders |
| Most Recent | |
| Episode Hypomanic | |
| Bipolar I Disorder, | Mood Disorders |
| Single Manic | |
| Episode, In Full | |
| Remission | |
| Bipolar I Disorder, | Mood Disorders |
| Single Manic | |
| Episode, In Partial | |
| Remission | |
| Bipolar I Disorder, | Mood Disorders |
| Single Manic | |
| Episode, Mild | |
| Bipolar I Disorder, | Mood Disorders |
| Single Manic | |
| Episode, Moderate | |
| Bipolar I Disorder, | Mood Disorders |
| Single Manic | |
| Episode, Severe With | |
| Psychotic Features | |
| Bipolar I Disorder, | Mood Disorders |
| Single Manic | |
| Episode, Severe | |
| Without Psychotic | |
| Features | |
| Bipolar I Disorder, | Mood Disorders |
| Single Manic | |
| Episode, Unspecified | |
| Bipolar II Disorder | Mood Disorders |
| Body Dysmorphic | Somatoform Disorders |
| Disorder | |
| Borderline Personality | Personality Disorders |
| Disorder | |
| Breathing-Related | Sleep Disorders, Dyssomnias |
| Sleep Disorder | |
| Brief Psychotic | Psychotic Disorders |
| Disorder | |
| Bulimia Nervosa | Eating Disorders |
| Circadian Rhythm | Sleep Disorders, Dyssomnias |
| Sleep Disorder | |
| Conversion Disorder | Somatoform Disorders |
| Cyclothymic Disorder | Mood Disorders |
| Delusional Disorder | Psychotic Disorders |
| Dependent | Personality Disorders |
| Personality Disorder | |
| Depersonalization | Dissociative Disorders |
| Disorder | |
| Depressive | Mood Disorders |
| Disorder NOS | |
| Dissociative Amnesia | Dissociative Disorders |
| Dissociative Disorder | Dissociative Disorders |
| NOS | |
| Dissociative Fugue | Dissociative Disorders |
| Dissociative Identity | Dissociative Disorders |
| Disorder | |
| Dyspareunia | Sexual Disorders, Sexual |
| | Dysfunctions |
| Dyssomnia NOS | Sleep Disorders, Dyssomnias |
| Dyssomnia Related to | Sleep Disorders |
| (Another | |
| Disorder) | |
| Dysthymic Disorder | Mood Disorders |
| Eating Disorder NOS | Eating Disorders |
| Exhibitionism | Sexual Disorders, Paraphilias |
| Female | Sexual Disorders, Sexual |
| Dyspareunia Due to | Dysfunctions |
| Medical | |
| Condition | |
| Female Hypoactive | Sexual Disorders, Sexual |
| Sexual Desire | |
| Disorder Due to | Dysfunctions |
| Medical Condition | |
| Female Orgasmic | Sexual Disorders, Sexual |
| Disorder | |
| | Dysfunctions |
| Female Sexual | Sexual Disorders, Sexual |
| Arousal Disorder | |
| | Dysfunctions |
| Fetishism | Sexual Disorders, Paraphilias |
| Frotteurism | Sexual Disorders, Paraphilias |
| Gender Identity | Sexual Disorders, Gender Identity |
| Disorder in | |
| Adolescents or Adults | Disorder |
| Gender Identity | Sexual Disorders, Gender Identity |
| Disorder in | |
| Children | Disorder |
| Gender Identity | Sexual Disorders, Gender Identity |
| Disorder NOS | |
| | Disorder |
| Generalized Anxiety | Anxiety Disorders |
| Disorder | |
| Histrionic Personality | Personality Disorders |
| Disorder | |
| Hypoactive Sexual | Sexual Disorders, Sexual |
| Desire Disorder | |
| | Dysfunctions |
| Hypochondriasis | Somatoform Disorders |
| Impulse -Control | Impulse-Control Disorders |
| Disorder NOS | |
| Insomnia Related to | Sleep Disorders |
| (Another | |
| Disorder) | |
| Intermittent Explosive | Impulse-Control Disorders |
| Disorder | |
| Kleptomania | Impulse-Control Disorders |
| Major Depressive | Mood Disorders |
| Disorder, | |
| Recurrent, In Full | |
| Remission | |
| Major Depressive | Mood Disorders |
| Disorder, | |
| Recurrent, In Partial | |
| Remission | |
| Major Depressive | Mood Disorders |
| Disorder, | |
| Recurrent, Mild | |
| Major Depressive | Mood Disorders |
| Disorder, | |
| Recurrent, Moderate | |
| Major Depressive | Mood Disorders |
| Disorder, | |
| Recurrent, Severe | |
| With Psychotic | |
| Features | |
| Major Depressive | Mood Disorders |
| Disorder, | |
| Recurrent, Severe | |
| Without Psychotic | |
| Features | |
| Major Depressive | Mood Disorders |
| Disorder, | |
| Recurrent, | |
| Unspecified | |
| Major Depressive | Mood Disorders |
| Disorder, Single | |
| Episode, In Full | |
| Remission | |
| Major Depressive | Mood Disorders |
| Disorder, Single | |
| Episode, In Partial | |
| Remission | |
| Major Depressive | Mood Disorders |
| Disorder, Single | |
| Episode, Mild | |
| Major Depressive | Mood Disorders |
| Disorder, Single | |
| Episode, Moderate | |
| Major Depressive | Mood Disorders |
| Disorder, Single | |
| Episode, Severe With | |
| Psychotic Features | |
| Major Depressive | Mood Disorders |
| Disorder, Single | |
| Episode, Severe | |
| Without Psychotic | |
| Features | |
| Major Depressive | Mood Disorders |
| Disorder, Single | |
| Episode, Unspecified | |
| Male | Sexual Disorders, Sexual |
| Dyspareunia Due to | Dysfunctions |
| Medical | |
| Condition | |
| Male Erectile Disorder | Sexual Disorders, Sexual |
| | Dysfunctions |
| Male Erectile Disorder | Sexual Disorders, Sexual |
| Due to | |
| Medical Condition | Dysfunctions |
| Male Hypoactive | Sexual Disorders, Sexual |
| Sexual Desire | |
| Disorder Due to | Dysfunctions |
| Medical Condition | |
| Male Orgasmic | Sexual Disorders, Sexual |
| Disorder | |
| | Dysfunctions |
| Mood Disorder Due to | Mood Disorders |
| Medical | |
| Condition | |
| Narcissistic | Personality Disorders |
| Personality Disorder | |
| Narcolepsy | Sleep Disorders, Dyssomnias |
| Nightmare Disorder | Sleep Disorders, Parasomnias |
| Obsessive | Anxiety Disorders |
| Compulsive Disorder | |
| Obsessive- | Personality Disorders |
| Compulsive | |
| Personality | |
| Disorder | |
| Other Female Sexual | Sexual Disorders, Sexual |
| Dysfunction | |
| Due to Medical | Dysfunctions |
| Condition | |
| Other Male Sexual | Sexual Disorders, Sexual |
| Dysfunction Due | |
| to Medical Condition | Dysfunctions |
| Pain Disorder | Somatoform Disorders |
| Associated with both | |
| Psychological Factors | |
| and Medical | |
| Conditions | |
| Pain Disorder | Somatoform Disorders |
| Associated with | |
| Psychological | |
| Features | |
| Panic Disorder with | Anxiety Disorders |
| Agoraphobia | |
| Panic Disorder | Anxiety Disorders |
| without Agoraphobia | |
| Paranoid Personality | Personality Disorders |
| Disorder | |
| Paraphilia, NOS | Sexual Disorders, Paraphilias |
| Parasomnia NOS | Sleep Disorders, Parasomnias |
| Pathological | Impulse-Control Disorders |
| Gambling | |
| Pedophilia | Sexual Disorders, Paraphilias |
| Personality Disorder | Personality Disorders |
| NOS | |
| Posttraumatic Stress | Anxiety Disorders |
| Disorder | |
| Premature Ejaculation | Sexual Disorders, Sexual |
| | Dysfunctions |
| Primary Hypersomnia | Sleep Disorders, Dyssomnias |
| Primary Insomnia | Sleep Disorders, Dyssomnias |
| Psychotic Disorder | Psychotic Disorders |
| Due to Medical | |
| Condition, with | |
| Delusions | |
| Psychotic Disorder | Psychotic Disorders |
| Due to Medical | |
| Condition, with | |
| Hallucinations | |
| Psychotic Disorder, | Psychotic Disorders |
| NOS | |
| Pyromania | Impulse-Control Disorders |
| Schizoaffective | Psychotic Disorders |
| Disorder | |
| Schizoid Personality | Personality Disorders |
| Disorder | |
| Schizophrenia, | Psychotic Disorders |
| Catatonic Type | |
| Schizophrenia, | Psychotic Disorders |
| Disorganized Type | |
| Schizophrenia, | Psychotic Disorders |
| Paranoid Type | |
| Schizophrenia, | Psychotic Disorders |
| Residual Type | |
| Schizophrenia, | Psychotic Disorders |
| Undifferentiated | |
| Type | |
| Schizophreniform | Psychotic Disorders |
| Disorder | |
| Schizotypal | Personality Disorders |
| Personality Disorder | |
| Sexual Aversion | Sexual Disorders, Sexual |
| Disorder | |
| | Dysfunctions |
| Sexual Disorder NOS | Sexual Disorders |
| Sexual Dysfunction | Sexual Disorders, Sexual |
| NOS | |
| | Dysfunctions |
| Sexual Masochism | Sexual Disorders, Paraphilias |
| Sexual Sadism | Sexual Disorders, Paraphilias |
| Shared Psychotic | Psychotic Disorders |
| Disorder | |
| Sleep Disorder Due to | Sleep Disorders |
| A Medical | |
| Condition, | |
| Hypersomnia Type | |
| Sleep Disorder Due to | Sleep Disorders |
| A Medical | |
| Condition, Insomnia | |
| Type | |
| Sleep Disorder Due to | Sleep Disorders |
| A Medical | |
| Condition, Mixed | |
| Type | |
| Sleep Disorder Due to | Sleep Disorders |
| A Medical | |
| Condition, | |
| Parasomnia Type | |
| Sleep Terror Disorder | Sleep Disorders, Parasomnias |
| Sleepwalking | Sleep Disorders, Parasomnias |
| Disorder | |
| Social Phobia | Anxiety Disorders |
| Somatization | Somatoform Disorders |
| Disorder | |
| Somatoform Disorder | Somatoform Disorders |
| NOS | |
| Specific Phobia | Anxiety Disorders |
| Transvestic Fetishism | Sexual Disorders, Paraphilias |
| Trichotillomania | Impulse-Control Disorders |
| Undifferentiated | Somatoform Disorders |
| Somatoform | |
| Disorder | |
| Vaginismus | Sexual Disorders, Sexual |
| | Dysfunctions |
| Voyeurism | Sexual Disorders, Paraphilias |

What is claimed is:

1. A computer-implemented method of directing mental exercise, comprising:
providing, by a first output component of a computing device, a stimulus representing an imagined perception, experience or activity that a user should attempt to generate in their mind;
providing, by a second output component of the computing device, an instruction for the user to perform a mental exercise comprising instructing the user to generate an internal felt sense of the imagined perception, experience or activity;
receiving, at a user interface of the computing device, an input that characterizes the user's internal felt sense, the input comprising an overt response from the user; determining, by a processing module of the computing device, an attribute of the received input;
determining, by the processing module of the computing device and based on the determined attribute, a next instruction;
storing at least one of the determined attribute and the determined next instruction in one or more memory locations of the computing device; and
training the user, comprising: (i) presenting the determined attribute, and (ii) providing, by the second output component, the next instruction.

2. The computer-implemented method of claim 1, wherein the stimulus is selected from the group consisting of an image, a video, a sound, and an animation.

3. The computer-implemented method of claim 1, wherein the input that characterizes the user's internal felt sense characterizes a time duration of the user's internal felt sense.

4. The computer-implemented method of claim 1, wherein the input that characterizes the user's internal felt sense characterizes an intensity of the user's internal felt sense.

5. The computer-implemented method of claim 1, wherein the input that characterizes the user's internal felt sense characterizes a satisfaction with the user's internal felt sense.

6. The computer-implemented method of claim 1, wherein the next instruction is provided repeatedly with less than 30 seconds elapsing between repetitions.

7. The computer-implemented method of claim 1, wherein the input that characterizes the user's internal felt sense is received at the user interface as selected from the group consisting of a selection of one or more buttons, a position of one or more sliders, one or more form input elements, a cursor position, a touch screen position, voice recognition, and one or more eye movements.

8. The computer-implemented method of claim 1, further comprising receiving, at the user interface, an input that characterizes the user, and selecting, based on the received input that characterizes the user, the stimulus from a plurality of predefined stimuli.

9. The computer-implemented method of claim 1, wherein the instruction for the user to perform a mental exercise is configured to decrease pain.

10. The computer-implemented method of claim 1, wherein the instruction for the user to perform a mental exercise is configured to decrease pain, decrease stress, treat depression, treat anxiety, treat addiction, treat insomnia decrease craving, increase attention, increase relaxation, increase happiness, increase focus, or increase learning.

11. The computer-implemented method of claim 1, wherein the mental exercise is capable of remaining internal to the mind of the user.

12. The computer-implemented method of claim 1, wherein the attribute comprises a score.

13. The computer-implemented method of claim 1, wherein the method is used with a medication therapy.

14. The computer-implemented method of claim 1, further comprising testing the mental exercise in combination with a plurality of medications, and identifying a particular medication from the plurality of medications to associate with the mental exercise.

15. The computer-implemented method of claim 1, wherein the user's internal felt sense includes a mental image.

16. The computer-implemented method of claim 1, wherein the mental exercise includes mental rehearsal.

17. The computer-implemented method of claim 1, wherein the imagined perception, experience or activity includes a first aspect followed in time by a second aspect, and wherein the instruction for the user to perform a mental exercise includes the instruction to generate the first aspect of the internal felt sense of the imagined perception, experience or activity, and then the second aspect of the internal felt sense of the imagined perception, experience or activity.

18. The computer-implemented method of claim 17, wherein a time between the first aspect and the second aspect is less than 10 seconds.

19. The computer-implemented method of claim 1, further comprising providing, on a display screen of the computing device, a moving object, wherein motion of the object is configured to guide timing of the mental exercise.

20. The computer-implemented method of claim 1, wherein each of the stimulus, instruction, and the mental exercise is derived based on brain imaging information.
